(19) United States
(12) Patent Application Publication
(10) Pub. No.: US 2023/0104311 A1
(43) Pub. Date: Apr. 6, 2023

(54) NOVEL 6-SUBSTITUTED 7-DEAZAPURINES AND CORRESPONDING NUCLEOSIDES AS MEDICAMENTS

(71) Applicant: KATHOLIEKE UNIVERSITEIT LEUVEN, Leuven (BE)

(72) Inventors: Piet HERDEWIJN, Heverlee (BE); Elisabetta GROAZ, Leuven (BE); Li QINGFENG, Leuven (BE)

(73) Assignee: KATHOLIEKE UNIVERSITEIT LEUVEN, Leuven (BE)

(21) Appl. No.: 17/799,695

(22) PCT Filed: Feb. 17, 2020
(86) PCT No.: PCT/EP2020/054104

§ 371 Date: Aug. 15, 2022

(51) Int. Cl. A61K 31/7064; C07D 487/04; C07H 19/14; A61P 31/14; A61P 31/16; B01J 31/22; B01J 31/30; B01J 27/128

(52) U.S. Cl. CPC A61K 31/7064; C07D 487/04; C07H 19/14; A61P 31/14; A61P 31/16; B01J 31/2213; B01J 31/30; B01J 27/128; B01J 2231/4277; B01J 2531/842

(57) ABSTRACT

The present invention relates to the synthesis of 6-substituted 7-deazapurines and their corresponding nucleosides by coupling aryl or alkyl Grignard reagents with halogenated purine nucleosides in the presence of iron or an iron/copper mixture such as Fe(acac)3/CuI. The present invention also relates to pharmaceutical compositions comprising said compounds and the use of said pharmaceutical compositions to treat or prevent viral infections.

### FIELD OF THE INVENTION

The present invention relates to a novel class of 6-substituted 7-deazapurines and their corresponding nucleosides and their use to treat and/or prevent viral infections and to manufacture a medicine to treat or prevent viral infections.

### BACKGROUND OF THE INVENTION

Purine nucleosides and their analogues display a wide range of biological activities. Several of these purine nucleosides are clinically used for treatment of cancers (e.g. fludarabine, cladribine, nelarabine and clofarabine) and viral infections (e.g. carbovir and adefovir). A particular series of purine nucleoside analogues that have not been systematically studied are 6-substituted 7-deazapurine nucleosides (tubercidin analogues), due to their difficult accessibility by chemical synthesis. As seen in Scheme 1, tubercidin (1) itself is a naturally occurring cytostatic antibiotic (Johnson, S. et al. Hematol. Oncol. 2000, 18(4), 141-153; Johnson, S. Expert Opin. Pharmacother. 2001 2(6), 929-943; Parker, W. B. et al. Curr. Opin. Invest. Drugs 2004 5, 592-596). The study of the synthesis of derivatives of tubercidin (Ramasamy, K. et al. Tetrahedron Lett. 1987, 28(43), 5107-5110; Tolman, R. et al. J. Am. Chem. Soc. 1969, 91(8), 2102-2108; Bergstrom, D. et al. J. Med. Chem. 1984, 27(3), 285-292; Wu, R. et al. J. Med. Chem. 2010, 53(22), 7958-7966) has led to the identification of 7-deazapurine nucleosides with antiviral (Wu, R. et al. J. Med. Chem. 2010, 53(22), 7958-7966), antibiotic (Anzai, K. et al. J. Antibiot. 1957, 10, 201-204) or cytostatic (Nauš, P. J. et al. Med. Chem. 2010, 53(1), 460-470; Perliková. P. et al. Bioorg. Med. Chem. 2011, 19(1):229-242; Nauš, P. et al. Bioorg. Med. Chem. 2012, 20(17), 5202-5214; Spáčilová, P. et al. ChemMedChem 2010, 5(8), 1386-1396; Bourderioux, A. et al. J. Med. Chem. 2011, 54(15), 5498-5507) activity.

For example, 7-thienyl-7-deazapurines ribonucleoside (2) showed cytostatic activity towards a wide panel of cancer cell lines (Bourderioux, A. et al. J. Med. Chem. 2011, 54(15), 5498-5507). Compound 3 was proven to be a potent inhibitor of poliovirus (PV) replication (IC₅₀=0.011 μM), and it is also a potent inhibitor of dengue virus (DENV2) replication (IC₅₀=0.039 μM) (Wu, R. et al. J. Med. Chem. 2010, 53(22), 7958-7966).

The 6-methyl-7-deazapurine ribonucleoside has been later found to display potent activity against hepatitis C virus (Nauš, P. et al. J. Med. Chem. 2014, 57(3), 1097-1110).

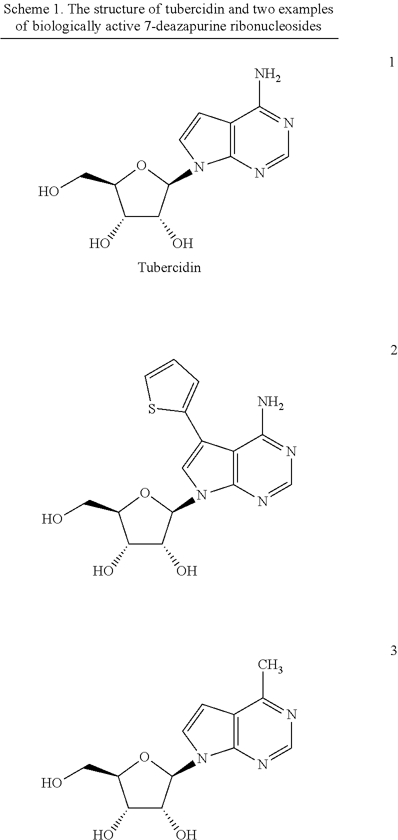 [SMILES: Nc1ncnc2c1ccn2[C@@H]1O[C@H](CO)[C@@H](O)[C@H]1O ; Nc1ncnc2c1c(-c1cccs1)cn2[C@@H]1O[C@H](CO)[C@@H](O)[C@H]1O ; Cc1ncnc2c1ccn2[C@@H]1O[C@H](CO)[C@@H](O)[C@H]1O]

Classical protocols for the synthesis of 6-substituted-7-deazapurine ribonucleosides rely on Suzuki-Miyaura cross-coupling reaction with palladium catalysts, aryl halides and organic boron compounds (Bourderioux, A. et al. J. Med. Chem. 2011, 54(15), 5498-5507). The synthesis of 6-methyl-7-deazapurine ribonucleoside was also carried out with trimethylaluminum as reagent and palladium as catalyst ((Wu, R. et al. J. Med. Chem. 2010, 53(22), 7958-7966; Nauš, P. et al. J. Med. Chem. 2014, 57(3), 1097-1110) (Scheme 2). These transformations require the presence of palladium or nickel as catalysts. These metals are costly or toxic and often necessitate sophisticated and expensive ligands of high molecular weight. Thus, there is a need for novel cheap and environmentally friendly catalysts that do not require complicated ligands to carry out such reactions. Additionally, it is well known that the amount of palladium in these modified nucleosides may lead to erroneous biological data when they are tested as potential antiviral and antitumoral compounds, which also highlights the need to find suitable alternatives to the use of palladium.

In recent years, iron catalysis has emerged as an increasing and promising alternative in many organic transformations, in particular for C—C bond-forming reactions, because of its low cost and toxicity (reviewed in Bedford, R. Acc. Chem. Res. 2015, 48(5), 1485-1493).

Since the pioneering works of Kochi in the 1970s, iron catalyzed cross-coupling reaction has been extensively studied. Fürstner et al. developed general conditions for cross-coupling reactions of alkyl and aryl Grignard reagents with aryl chlorides (Fürstner, A. et al. Angewandte Chemie 2002, 114 (4), 632-635 and J. Am. Chem. Soc. 2002, 124 (46), 13856-13863. Unlike aryl chlorides, the corresponding bromides and iodides are prone to reduction of the C—X bonds due to a radical decomposition pathway.

The first application in the nucleoside field was described by Hoeck et al. The authors achieved introduction of a methyl group by using CH₃MgBr as reagent and Fe(acac)₃ as catalyst on 2,6-dichloropurine and 2,6,8-trichloropurine (Hocek, M. et al. J. Org. Chem. 2003, 68 (14), 5773-5776; Hocek, M. et al. Synthesis 2004, (17), 2869-2876.

The present application relates to a novel method for coupling of 6-chloro-7-deazapurine and its ribonucleoside. Suitably, the inventors have found that the method can be used to couple a wide variety of functionalized aryl and alkyl Grignard reagents by using iron/copper bimetallic catalysts, leading to a series of 6-substituted-7-deazapurine nucleoside analogues.

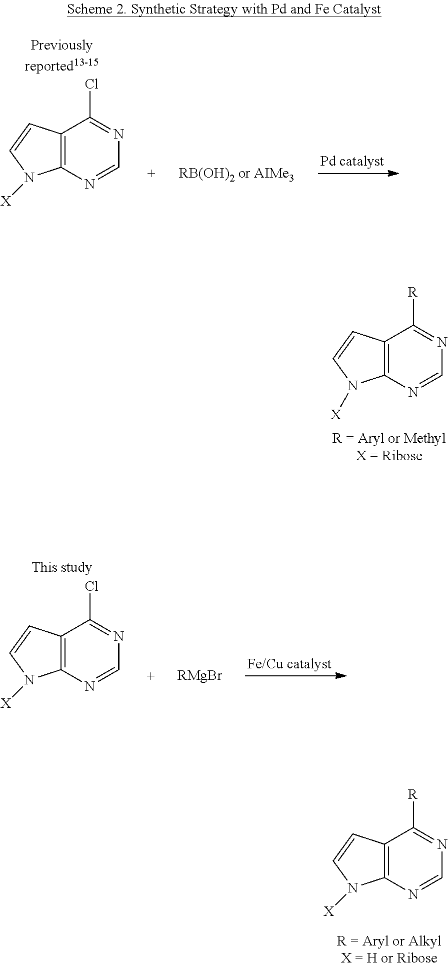

### SUMMARY OF THE INVENTION

The present invention relates to compounds of the general formula (A) for use as a medicament, wherein in general formula (A)

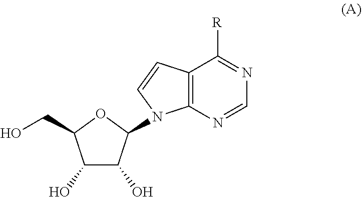

R is: (i) a straight or branched, saturated or unsaturated alkyl group; (ii) a cycloalkyl group; (iii) a straight or branched aryl group; (iv) an alkylaryl group; (v) an alkoxyaryl group; or (vi) an alkylaminoaryl group.

In one aspect of the invention, the compounds are for use in the prevention and/or treatment of viral infections in a mammal.

In some embodiments, the virus is an RNA-virus.

In preferred embodiments, the mammal is a human.

In some embodiments, the virus is a Human Norovirus (HuNoV); in some embodiments, the Human Norovirus belongs to the Human Norovirus Genogroup 1.

In some embodiments, the compound or the pharmaceutically acceptable salt thereof has a general formula (A), wherein R is an alkyl group with up to 6 carbon atoms.

In some specific embodiments, R is a C1-4 alkyl group.

In some embodiments, the compound or the pharmaceutically acceptable salt thereof has a general formula (A), wherein R is an alkylaryl group.

In some specific embodiments the alkyl group in the alkylaryl group is a C1-3 alkyl group.

In some embodiments, the compound for use in the invention may be selected from the group represented below:

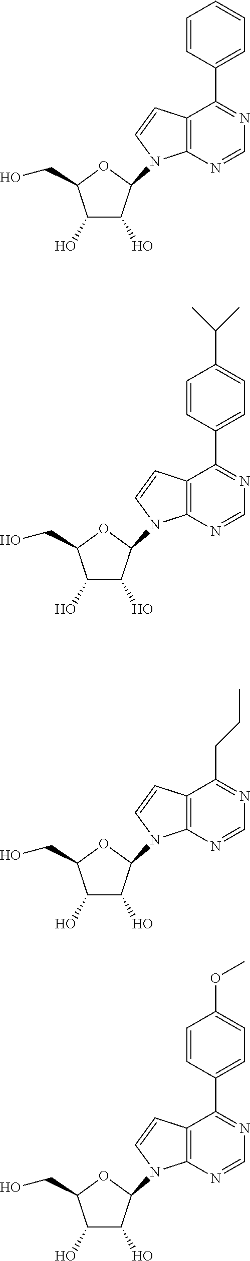 [SMILES: OC[C@H]1O[C@@H](n2ccc3c(-c4ccccc4)ncnc32)[C@H](O)[C@@H]1O ; CC(C)c1ccc(-c2ncnc3c2ccn3[C@@H]2O[C@H](CO)[C@@H](O)[C@H]2O)cc1 ; CCCc1ncnc2c1ccn2[C@@H]1O[C@H](CO)[C@@H](O)[C@H]1O ; COc1ccc(-c2ncnc3c2ccn3[C@@H]2O[C@H](CO)[C@@H](O)[C@H]2O)cc1]

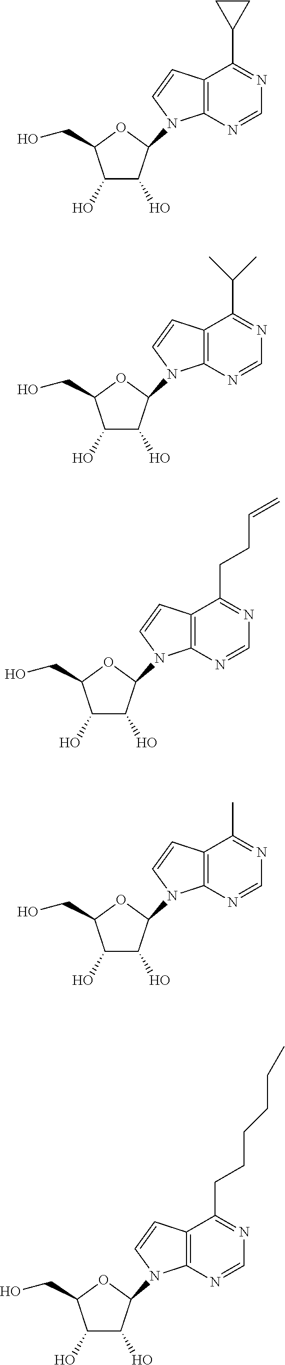 [SMILES: OC[C@H]1O[C@@H](n2ccc3c(C4CC4)ncnc32)[C@H](O)[C@@H]1O ; CC(C)c1ncnc2c1ccn2[C@@H]1O[C@H](CO)[C@@H](O)[C@H]1O ; C=CCCc1ncnc2c1ccn2[C@@H]1O[C@H](CO)[C@@H](O)[C@H]1O ; Cc1ncnc2c1ccn2[C@@H]1O[C@H](CO)[C@@H](O)[C@H]1O ; CCCCCCc1ncnc2c1ccn2[C@@H]1O[C@H](CO)[C@@H](O)[C@H]1O]

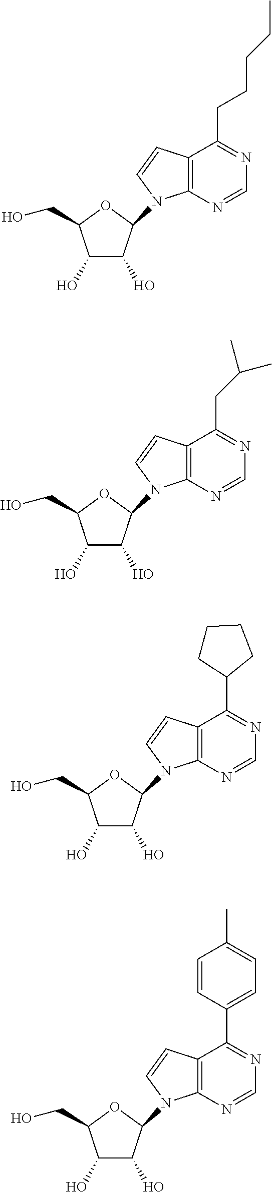 [SMILES: CCCCCc1ncnc2c1ccn2[C@@H]1O[C@H](CO)[C@@H](O)[C@H]1O ; CC(C)Cc1ncnc2c1ccn2[C@@H]1O[C@H](CO)[C@@H](O)[C@H]1O ; OC[C@H]1O[C@@H](n2ccc3c(C4CCCC4)ncnc32)[C@H](O)[C@@H]1O ; Cc1ccc(-c2ncnc3c2ccn3[C@@H]2O[C@H](CO)[C@@H](O)[C@H]2O)cc1]

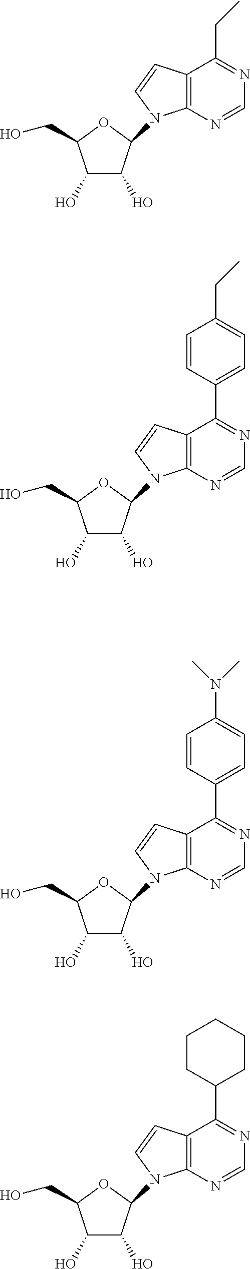 [SMILES: CCc1ncnc2c1ccn2[C@@H]1O[C@H](CO)[C@@H](O)[C@H]1O ; CCc1ccc(-c2ncnc3c2ccn3[C@@H]2O[C@H](CO)[C@@H](O)[C@H]2O)cc1 ; CN(C)c1ccc(-c2ncnc3c2ccn3[C@@H]2O[C@H](CO)[C@@H](O)[C@H]2O)cc1 ; OC[C@H]1O[C@@H](n2ccc3c(C4CCCCC4)ncnc32)[C@H](O)[C@@H]1O]

In some specific embodiments, the compound for use in the invention has the formula (B):

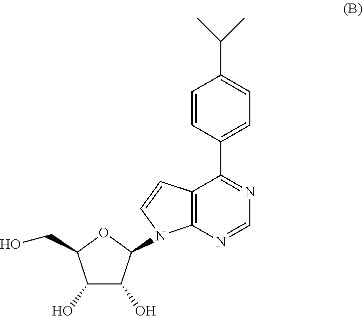 [SMILES: CC(C)c1ccc(-c2ncnc3c2ccn3[C@@H]2O[C@H](CO)[C@@H](O)[C@H]2O)cc1]

In other specific embodiments, the compound for use in the invention has the formula (C):

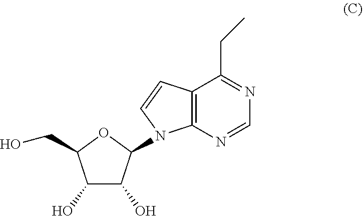 [SMILES: CCc1ncnc2c1ccn2[C@@H]1O[C@H](CO)[C@@H](O)[C@H]1O]

In some embodiments of the invention, the compound for use in the invention is the compound of formula (C) and the virus is a MERS coronavirus.

In some embodiments of the invention, the compound for use in the invention is the compound of formula (C) and the virus is Influenza A.

A second aspect of the invention relates to a pharmaceutical composition for inhibiting viral infection in a mammal, comprising: (i) a therapeutically effective amount of the compound of general formula (A) and/or a pharmaceutical acceptable addition salt thereof and/or a stereoisomer thereof and/or a solvate thereof; and (ii) at least one pharmaceutically acceptable carrier.

A third aspect of the invention relates to the use of the compound of the first aspect of the invention or the pharmaceutical composition of the second aspect of the invention for the manufacture of a medicament for the prevention or treatment of a viral infection in a mammal.

A fourth aspect of the invention relates to a method for prevention or treatment of a viral infection in a mammal, comprising providing to said subject a therapeutically effective amount of the compound of the first aspect of the invention.

A fifth aspect of the invention relates to a kit of parts comprising (i) the compound or pharmaceutical composition of the first and second aspects of the invention and (ii) instructions for use.

A sixth aspect of the invention relates to a compound of the general formula (A), wherein in general formula (A) R is: (i) a straight or branched, saturated or unsaturated alkyl group; (ii) a cycloalkyl group; (iii) a straight or branched aryl group; (iv) an alkylaryl group; (v) an alkoxyaryl group; or (vi) an alkylaminoaryl group; and wherein the compound does not comprise the compounds having the formula below.

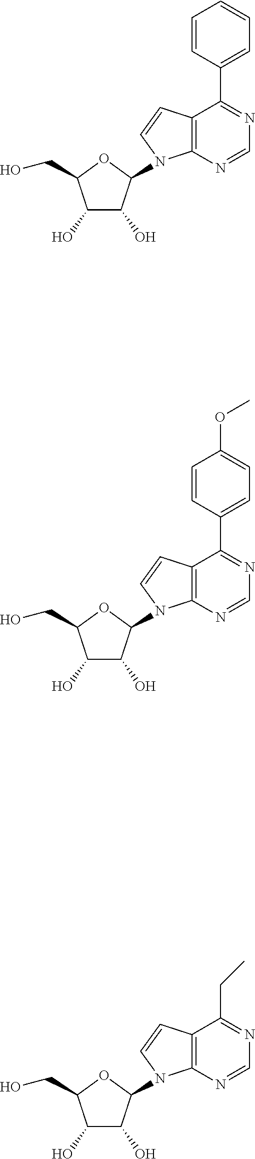 [SMILES: OC[C@H]1O[C@@H](n2ccc3c(-c4ccccc4)ncnc32)[C@H](O)[C@@H]1O ; COc1ccc(-c2ncnc3c2ccn3[C@@H]2O[C@H](CO)[C@@H](O)[C@H]2O)cc1 ; CCc1ncnc2c1ccn2[C@@H]1O[C@H](CO)[C@@H](O)[C@H]1O]

A seventh aspect of the invention relates to a method for synthesizing purine modified nucleoside analogues comprising a cross-coupling reaction of aryl or alkyl Grignard reagents with halogenated purine nucleosides, wherein the catalyst in the cross-coupling reaction is: (i) iron; or (ii) an iron/copper mixture.

In some embodiments, the purine modified nucleoside analogue is a pyrrolopyrimidine modified nucleoside analogue.

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

### DETAILED DESCRIPTION OF THE INVENTION

The present invention relates to a compound of the general formula (A) for use as a medicament, wherein in general formula (A)

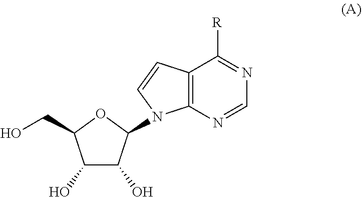

R is: (i) an alkyl group; (ii) a cycloalkyl group; (iii) an aryl group; (iv) an alkylaryl group; (v) an alkoxyaryl group; or (vi) an alkylaminoaryl group.

The compounds of the invention may be for use in the prevention and/or treatment of viral infections in a mammal.

In some embodiments of the invention, the virus is an RNA-virus.

In some embodiments, the mammal is a human.

Human diseases causing RNA viruses include Noroviruses, Orthomyxoviruses, Hepatitis C Virus (HCV), Ebola disease, SARS, influenza, dengue, Zika, respiratory syncytial virus, yellow fever, polio measles and retrovirus including adult Human T-cell lymphotropic virus type 1 (HTLV-1) and human immunodeficiency virus (HIV). RNA viruses have RNA as genetic material, that may be a single-stranded RNA or a double stranded RNA. Viruses may exploit the presence of RNA-dependent RNA polymerases for replication of their genomes or, in retroviruses, with two copies of single strand RNA genomes, reverse transcriptase produces viral DNA which can be integrated into the host DNA under its integrase function. Studies showed that endogenous retroviruses are long-terminal repeat (LTR)-type retroelements that account for approximately 10% of human or murine genomic DNA.

In some embodiments, the virus is a Norovirus.

Noroviruses are positive-sense single-stranded RNA (+ssRNA) viruses responsible for large outbreaks of acute gastroenteritis around the world (Patel, M. M. et al. J. Clin. Virol. 2009, 44, 1-8; Glass, R. I. et al; New Engl. J. Med. 2009, 361, 1776-1785). Typically, symptoms including vomiting, diarrhea, abdominal cramps, and nausea may last about two or three days in healthy adults. However, this infection can be prolonged and severe (even life-threatening) in young children, elderly and immunocompromised persons, who can develop chronic grastroenteritis (Payne, D. C. et al. New Engl. J. Med. 2013, 368, 1121-1130; Bok, K. et al. New Engl. J. Med. 2012, 367, 2126-2132).

The current therapy for norovirus infections relies on electrolyte replenishment for dehydrated individuals along with measures for outbreak control and prevention that are restricted to the often inefficient use of antiseptics and hand-sanitizers. Despite the clear need for medical intervention, no approved vaccine or small molecule antiviral treatment is currently available. This is partly due to the fact that only recently human norovirus (HuNoV) was successfully cultivated in vitro (Duizer, E. et al. J. Gen. Virol. 2004, 85, 79-87; Lay, M. K. et al. Virology 2010, 406, 1-11). Hence, there is an urgency to develop therapeutic agents that can either directly inhibit norovirus RNA replication or interfere with the function of structural and non-structural proteins encoded by the norovirus genome (Bassetto, M. et al. Viruses 2019, 11, doi:10.3390/v11020173; Weerasekara, S. et al. Expert Opin. Drug Dis. 2016, 11, 529-541). Selected examples of molecules recognized as inhibitors of the activity of norovirus RNA-dependent RNA polymerase (RdRp) are given in FIG. 2. From a structural standpoint, they include both nucleosides and non-nucleoside compounds (Netzler, N. E. et al. Med. Res. Rev. 2019, 39, 860-886; Costantini, V. P. et al. Antivir. Ther. 2012, 17, 981-991).

Several small heterocycles exhibiting anti-norovirus inhibitory activity in the micromolar range, such as the phenylthiazole (NIC02) and triazole (NIC10) derivatives were identified as potential scaffolds for further drug development (Eltahla, A. A. et al. Antimicrob. Agents Chemother. 2014, 58, 3115-3123). Ribavirin was one of the first nucleosides found to effectively inhibit norovirus replication (EC50=43 μM) (Chang, K. O. et al. J. Virol. 2007, 81, 12111-12118). 2′-C-Methyl-cytidine (2CM-C) was initially developed as a HCV polymerase inhibitor, but later proven to inhibit also murine norovirus (MNV) polymerase (EC50=2 μM) (Rocha-Pereira, J. et al. Biochem. Biophys. Res. Commun. 2012, 427, 796-800) and HuNoV replication in the human B cell BJAB cell line (EC50=0.3 μM) (Kolawole, A. O. et al. Antivir. Res. 2016, 132, 46-49).

Both 2CM-C and its fluorinated analogue 2′-fluoro-2′-C-methyl-cytidine (2′-F-2′-CM-C) displayed comparable antiviral activity against both MNV and HuNoV in cell based assays. Among non-nucleoside anti-norovirus agents, it is worth mentioning polyanionic naphthalene analogues such as suramin (Mastrangelo, E. et al. Chemmedchem 2014, 9, 933-939; Mastrangelo, E. et al. J. Mol. Biol. 2012, 419, 198-210) and NF023 16.

In recent years, much effort has been devoted to the chemical synthesis and biological evaluation of C-nucleoside analogues as potential antiviral agents (De Clercq, E. J. Med. Chem. 2016, 59, 2301-2311; Temburnikar, K. et al. J. Org. Chem. 2018, 14, 772-785). In particular, the coupling of 4-amino-pyrrolo[2,1-f][1,2,4]triazine (or 4-aza-7,9-dideazaadenine) to various sugar moieties has delivered modified C-nucleosides with a broad-spectrum activity against viruses belonging to the Flaviviridae (HCV), Orthomyxoviridae, Paramyxoviridae, and Coronaviridae families. In an early study, 2′-C-methyl-4-aza-7,9-dideazaadenosine (FIG. 3, 1) was identified as a selective HCV polymerase inhibitor in cell cultures (EC50=1.98 μM) (Cho, A. et al. Bioorg. Med. Chem. Lett. 2012, 22, 4127-4132).

Base-modified derivatives of 1 have also shown promising anti-HCV properties in vitro. Specifically, the 7-carboamido analogue 2 exhibited a remarkable 40-fold improvement in potency relative to the parent compound 1, while the 7-fluoro pyrrolotriazine analogue 3 showed good anti-HCV activity (EC50=3.1 μM) without concomitant cytotoxicity (CC50>100 μM) (Draffan, A. G. et al. Bioorg. Med. Chem. Lett. 2014, 24, 4984-4988). The and ribose analogues containing C-nucleosides 4-7 with either a hydrogen atom or halogen group at the 7-position of the nucleobase were subsequently synthetized by the present inventors (Li, Q. F. et al. Chemmedchem 2018, 13, 97-104).

Currently, at least thirty-three different norovirus genotypes have been described. Human Noroviruses may belong to three genogroups: GI, GII, and GIV. In some embodiments of the invention, the Human Norovirus belongs to the Human Norovirus Genogroup 1.

In some embodiments, the compound or the pharmaceutically acceptable salt thereof has a general formula (A), wherein R is an alkyl group with up to 6 carbon atoms.

In some specific embodiments, R is a C1-4 alkyl group.

In some embodiments, the compound or the pharmaceutically acceptable salt thereof has a general formula (A), wherein R is an aryl group.

In some specific embodiments, the compound or the pharmaceutically acceptable salt thereof has a general formula (A), wherein R is a phenyl.

In some embodiments, the compound or the pharmaceutically acceptable salt thereof has a general formula (A), wherein R is an alkylaryl group.

In some embodiments the alkyl group in the alkylaryl group is a C1-3 alkyl group.

In some embodiments the aryl group in the alkylaryl group is a substituted phenyl.

In specific embodiments, the alkylaryl group is a substituted phenol with a C1-3 alkyl group.

In some embodiments, the compound for use in the invention is selected from the group represented below:

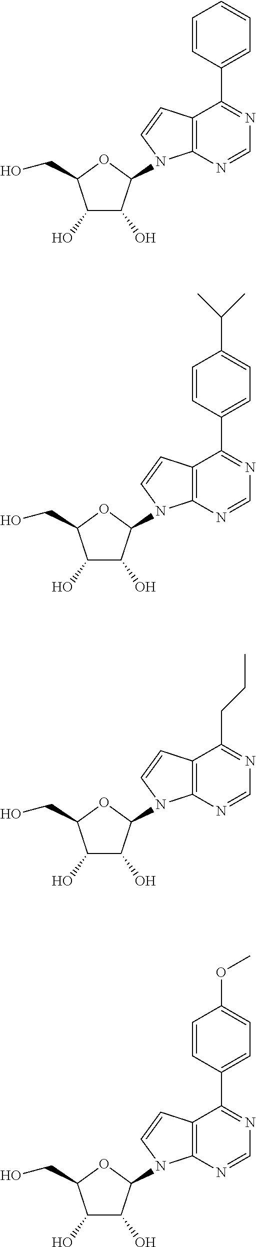 [SMILES: OC[C@H]1O[C@@H](n2ccc3c(-c4ccccc4)ncnc32)[C@H](O)[C@@H]1O ; CC(C)c1ccc(-c2ncnc3c2ccn3[C@@H]2O[C@H](CO)[C@@H](O)[C@H]2O)cc1 ; CCCc1ncnc2c1ccn2[C@@H]1O[C@H](CO)[C@@H](O)[C@H]1O ; COc1ccc(-c2ncnc3c2ccn3[C@@H]2O[C@H](CO)[C@@H](O)[C@H]2O)cc1]

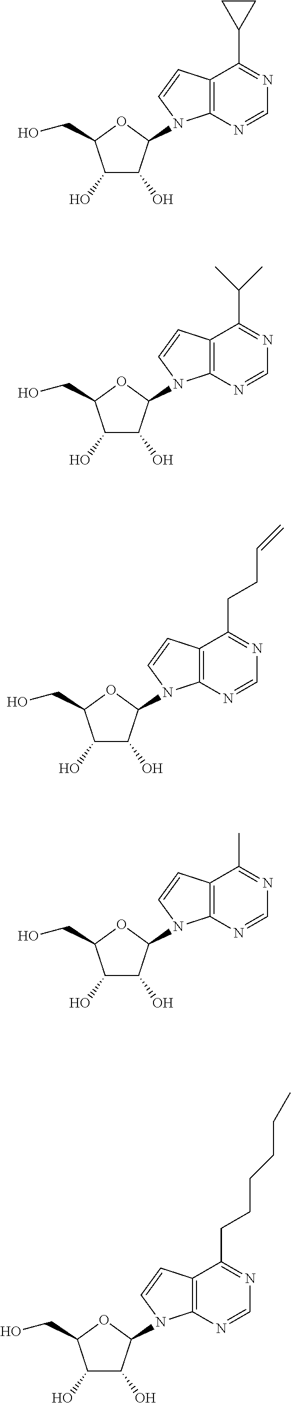 [SMILES: OC[C@H]1O[C@@H](n2ccc3c(C4CC4)ncnc32)[C@H](O)[C@@H]1O ; CC(C)c1ncnc2c1ccn2[C@@H]1O[C@H](CO)[C@@H](O)[C@H]1O ; C=CCCc1ncnc2c1ccn2[C@@H]1O[C@H](CO)[C@@H](O)[C@H]1O ; Cc1ncnc2c1ccn2[C@@H]1O[C@H](CO)[C@@H](O)[C@H]1O ; CCCCCCc1ncnc2c1ccn2[C@@H]1O[C@H](CO)[C@@H](O)[C@H]1O]

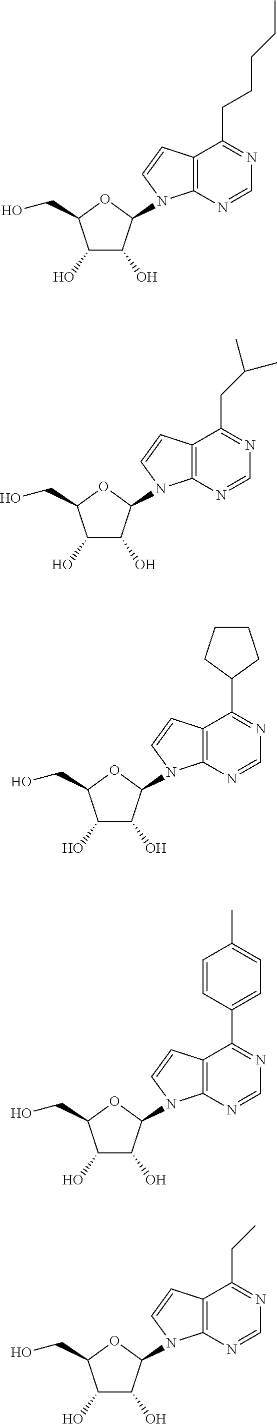 [SMILES: CCCCCc1ncnc2c1ccn2[C@@H]1O[C@H](CO)[C@@H](O)[C@H]1O ; CC(C)Cc1ncnc2c1ccn2[C@@H]1O[C@H](CO)[C@@H](O)[C@H]1O ; OC[C@H]1O[C@@H](n2ccc3c(C4CCCC4)ncnc32)[C@H](O)[C@@H]1O ; Cc1ccc(-c2ncnc3c2ccn3[C@@H]2O[C@H](CO)[C@@H](O)[C@H]2O)cc1 ; CCc1ncnc2c1ccn2[C@@H]1O[C@H](CO)[C@@H](O)[C@H]1O]

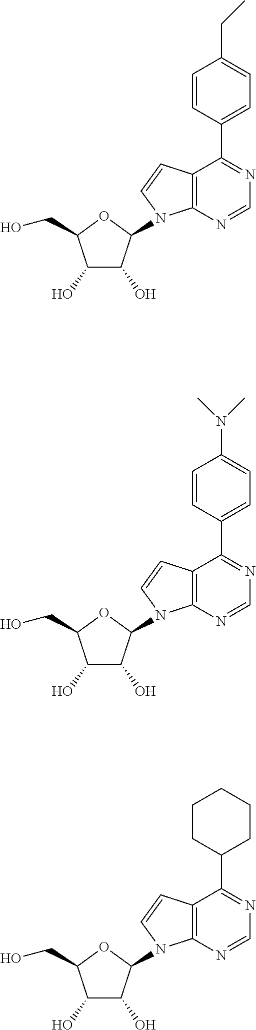 [SMILES: CCc1ccc(-c2ncnc3c2ccn3[C@@H]2O[C@H](CO)[C@@H](O)[C@H]2O)cc1 ; CN(C)c1ccc(-c2ncnc3c2ccn3[C@@H]2O[C@H](CO)[C@@H](O)[C@H]2O)cc1 ; OC[C@H]1O[C@@H](n2ccc3c(C4CCCCC4)ncnc32)[C@H](O)[C@@H]1O]

In some embodiments, the compound for use in the invention is selected from the group represented below:

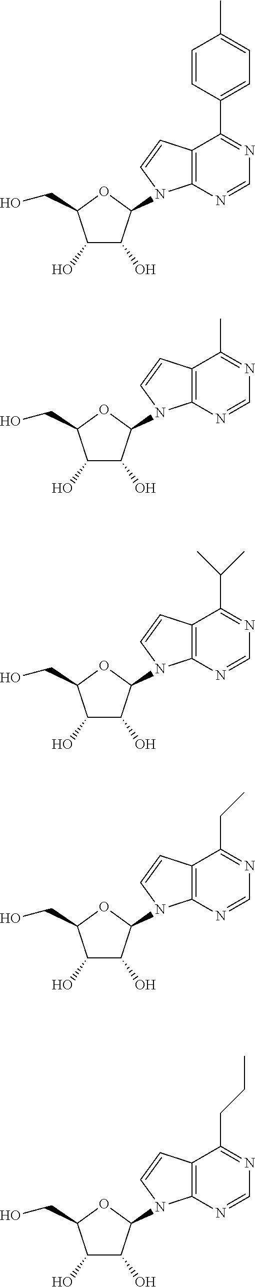 [SMILES: Cc1ccc(-c2ncnc3c2ccn3[C@@H]2O[C@H](CO)[C@@H](O)[C@H]2O)cc1 ; Cc1ncnc2c1ccn2[C@@H]1O[C@H](CO)[C@@H](O)[C@H]1O ; CC(C)c1ncnc2c1ccn2[C@@H]1O[C@H](CO)[C@@H](O)[C@H]1O ; CCc1ncnc2c1ccn2[C@@H]1O[C@H](CO)[C@@H](O)[C@H]1O ; CCCc1ncnc2c1ccn2[C@@H]1O[C@H](CO)[C@@H](O)[C@H]1O]

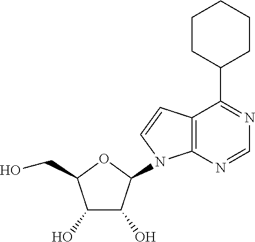 [SMILES: OC[C@H]1O[C@@H](n2ccc3c(C4CCCCC4)ncnc32)[C@H](O)[C@@H]1O]

In some specific embodiments, the compound for use in the invention is selected from the group represented below:

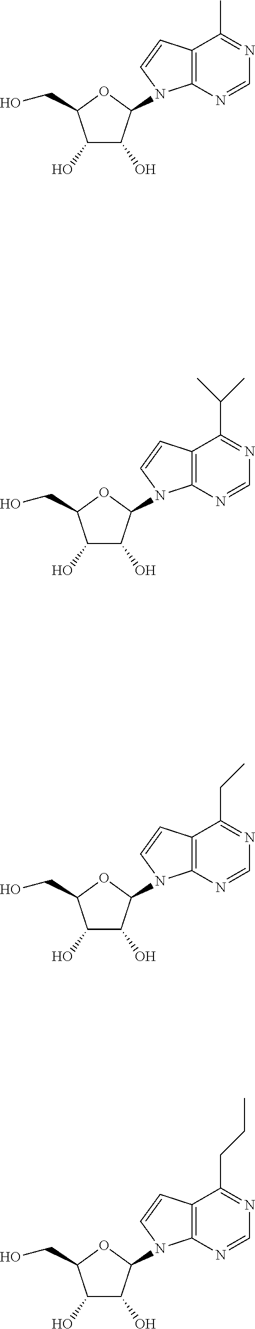 [SMILES: Cc1ncnc2c1ccn2[C@@H]1O[C@H](CO)[C@@H](O)[C@H]1O ; CC(C)c1ncnc2c1ccn2[C@@H]1O[C@H](CO)[C@@H](O)[C@H]1O ; CCc1ncnc2c1ccn2[C@@H]1O[C@H](CO)[C@@H](O)[C@H]1O ; CCCc1ncnc2c1ccn2[C@@H]1O[C@H](CO)[C@@H](O)[C@H]1O]

In some embodiments, the compound for use in the invention has the formula (B):

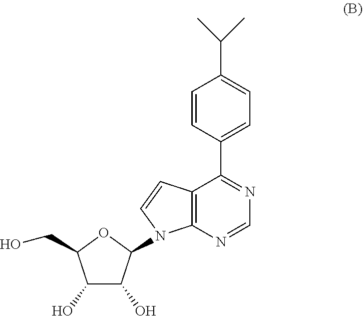 [SMILES: CC(C)c1ccc(-c2ncnc3c2ccn3[C@@H]2O[C@H](CO)[C@@H](O)[C@H]2O)cc1]

In other embodiments, the compound for use in the invention has the formula (C):

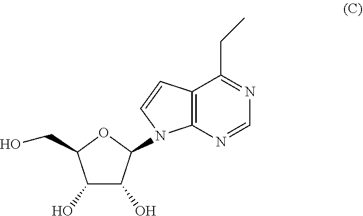 [SMILES: CCc1ncnc2c1ccn2[C@@H]1O[C@H](CO)[C@@H](O)[C@H]1O]

In some embodiments, the compound for use in the invention is the compound of formula (C) and the virus is a MERS coronavirus.

As a novel coronavirus first reported by Saudi Arabia in 2012, the Middle East respiratory syndrome coronavirus (MERS-CoV) is responsible for an acute human respiratory syndrome. The virus, of 2C beta-CoV lineage, expresses the dipeptidyl peptidase 4 (DPP4) receptor and is densely endemic in dromedary camels of East Africa and the Arabian Peninsula. MERS-CoV is zoonotic but human-to-human transmission is also possible. Surveillance and phylogenetic researches indicate MERS-CoV to be closely associated with bats' coronaviruses, suggesting bats as reservoirs, although unconfirmed. With no vaccine currently available for MERS-CoV nor approved prophylactics, its global spread to over 25 countries with high fatalities highlights its role as ongoing public health threat. There is thus a clear need for an articulated action plan for advanced countermeasures against MERS-CoV (reviewed in Ramadan N. et al., Germs. 2019 March; 9(1): 35-42).

In other embodiments, the compound for use in the invention is the compound of formula (C) and the virus is Influenza A.

Influenza A viruses are negative-sense, single-stranded, segmented RNA viruses. The several subtypes are labeled according to an H number (for the type of hemagglutinin) and an N number (for the type of neuraminidase). There are 18 different known H antigens (H1 to H18) and 11 different known N antigens (N1 to N11). Each virus subtype has mutated into a variety of strains with differing pathogenic profiles; some are pathogenic to one species but not others, some are pathogenic to multiple species (“Influenza Type A Viruses and Subtypes”, Centers for Disease Control and Prevention).

A second aspect of the invention relates to a pharmaceutical composition for inhibiting viral infection in a mammal, comprising: (i) a therapeutically effective amount of the compound of any one of the preceding claims and/or a pharmaceutical acceptable addition salt thereof and/or a stereoisomer thereof and/or a solvate thereof; and (ii) at least one pharmaceutically acceptable carrier.

The term “pharmaceutically acceptable carrier or excipient” as used herein in relation to pharmaceutical compositions and combined preparations means any material or substance with which the active principle i.e. the compounds of general formula (A), and optionally an antiviral agent and/or an immunosuppressant or immunomodulator may be formulated in order to facilitate its application or dissemination to the locus to be treated, for instance by dissolving, dispersing or diffusing said composition, and/or to facilitate its storage, transport or handling without impairing its effectiveness. The pharmaceutically acceptable carrier may be a solid or a liquid or a gas which has been compressed to form a liquid, i.e. the compositions of this invention can suitably be used as concentrates, emulsions, solutions, granulates, dusts, sprays, aerosols, pellets or powders. Suitable pharmaceutical carriers for use in said pharmaceutical compositions and their formulation are well known to those skilled in the art. There is no particular restriction to their selection within the present invention. Suitable pharmaceutical carriers include additives such as wetting agents, dispersing agents, stickers, adhesives, emulsifying or surface-active agents, thickening agents, complexing agents, gelling agents, solvents, coatings, antibacterial and antifungal agents (for example phenol, sorbic acid, chlorobutanol), isotonic agents (such as sugars or sodium chloride) and the like, provided the same are consistent with pharmaceutical practice, i.e. carriers and additives which do not create permanent damage to mammals.

The pharmaceutical compositions of the present invention may be prepared in any known manner, for instance by homogeneously mixing, dissolving, spray-drying, coating and/or grinding the active ingredients, in a one-step or a multi-steps procedure, with the selected carrier material and, where appropriate, the other additives such as surface-active agents, may also be prepared by micronisation, for instance in view to obtain them in the form of microspheres usually having a diameter of about 1 to 10 μm, namely for the manufacture of microcapsules for controlled or sustained release of the biologically active ingredient(s).

Suitable surface-active agents to be used in the pharmaceutical compositions of the present invention are non-ionic, cationic and/or anionic surfactants having good emulsifying, dispersing and/or wetting properties. Suitable anionic surfactants include both water-soluble soaps and water-soluble synthetic surface-active agents. Suitable soaps are alkaline or alkaline-earth metal salts, unsubstituted or substituted ammonium salts of higher fatty acids (C₁₀-C₂₂), e.g. the sodium or potassium salts of oleic or stearic acid, or of natural fatty acid mixtures obtainable form coconut oil or tallow oil. Synthetic surfactants include sodium or calcium salts of polyacrylic acids; fatty sulphonates and sulphates; sulphonated benzimidazole derivatives and alkylarylsulphonates. Fatty sulphonates or sulphates are usually in the form of alkaline or alkaline-earth metal salts, unsubstituted ammonium salts or ammonium salts substituted with an alkyl or acyl radical having from 8 to 22 carbon atoms, e.g. the sodium or calcium salt of lignosulphonic acid or dodecylsulphonic acid or a mixture of fatty alcohol sulphates obtained from natural fatty acids, alkaline or alkaline-earth metal salts of sulphuric or sulphonic acid esters (such as sodium lauryl sulphate) and sulphonic acids of fatty alcohol/ethylene oxide adducts. Suitable sulphonated benzimidazole derivatives preferably contain 8 to 22 carbon atoms. Examples of alkylarylsulphonates are the sodium, calcium or alcanolamine salts of dodecylbenzene sulphonic acid or dibutyl-naphtalenesulphonic acid or a naphthalene-sulphonic acid/formaldehyde condensation product. Also suitable are the corresponding phosphates, e.g. salts of phosphoric acid ester and an adduct of p-nonylphenol with ethylene and/or propylene oxide, or phospholipids. Suitable phospholipids for this purpose are the natural (originating from animal or plant cells) or synthetic phospholipids of the cephalin or lecithin type such as e.g. phosphatidylethanolamine, phosphatidylserine, phosphatidylglycerine, lysolecithin, cardiolipin, dioctanyl-phosphatidylcholine, dipalmitoylphoshatidylcholine and their mixtures.

Suitable non-ionic surfactants include polyethoxylated and polypropoxylated derivatives of alkylphenols, fatty alcohols, fatty acids, aliphatic amines or amides containing at least 12 carbon atoms in the molecule, alkylarenesulphonates and dialkylsulphosuccinates, such as polyglycol ether derivatives of aliphatic and cycloaliphatic alcohols, saturated and unsaturated fatty acids and alkylphenols, said derivatives preferably containing 3 to 10 glycol ether groups and 8 to 20 carbon atoms in the (aliphatic) hydrocarbon moiety and 6 to 18 carbon atoms in the alkyl moiety of the alkylphenol. Further suitable non-ionic surfactants are water-soluble adducts of polyethylene oxide with poylypropylene glycol, ethylenediamino-polypropylene glycol containing 1 to 10 carbon atoms in the alkyl chain, which adducts contain 20 to 250 ethyleneglycol ether groups and/or 10 to 100 propyleneglycol ether groups. Such compounds usually contain from 1 to 5 ethyleneglycol units per propyleneglycol unit. Representative examples of non-ionic surfactants are nonylphenol-polyethoxyethanol, castor oil polyglycolic ethers, polypropylene/polyethylene oxide adducts, tributylphenoxypolyethoxyethanol, polyethyleneglycol and octylphenoxypolyethoxyethanol. Fatty acid esters of polyethylene sorbitan (such as polyoxyethylene sorbitan trioleate), glycerol, sorbitan, sucrose and pentaerythritol are also suitable non-ionic surfactants.

Suitable cationic surfactants include quaternary ammonium salts, preferably halides, having four hydrocarbon radicals optionally substituted with halo, phenyl, substituted phenyl or hydroxy; for instance quaternary ammonium salts containing as N-substituent at least one C₈-C₂₂ alkyl radical (e.g. cetyl, lauryl, palmityl, myristyl, oleyl and the like) and, as further substituents, unsubstituted or halogenated lower alkyl, benzyl and/or hydroxy-C₁₋₄ alkyl radicals. A more detailed description of surface-active agents suitable for this purpose may be found for instance in “McCutcheon's Detergents and Emulsifiers Annual” (MC Publishing Crop., Ridgewood, N.J., 1981), “Tensid-Taschenbuch”, 2^{nd} ed. (Hanser Verlag, Vienna, 1981) and “Encyclopaedia of Surfactants” (Chemical Publishing Co., New York, 1981). Structure-forming, thickening or gel-forming agents may be included into the pharmaceutical compositions and combined preparations of the invention. Suitable such agents are in particular highly dispersed silicic acid, such as the product commercially available under the trade name Aerosil; bentonites; tetraalkyl ammonium salts of montmorillonites (e.g., products commercially available under the trade name Bentone), wherein each of the alkyl groups may contain from 1 to 20 carbon atoms; cetostearyl alcohol and modified castor oil products (e.g. the product commercially available under the trade name Antisettle).

Gelling agents which may be included into the pharmaceutical compositions and combined preparations of the present invention include, but are not limited to, cellulose derivatives such as carboxymethylcellulose, cellulose acetate and the like; natural gums such as arabic gum, xanthum gum, tragacanth gum, guar gum and the like; gelatin; silicon dioxide; synthetic polymers such as carbomers, and mixtures thereof. Gelatin and modified celluloses represent a preferred class of gelling agents.

Other optional excipients which may be included in the pharmaceutical compositions and combined preparations of the present invention include additives such as magnesium oxide; azo dyes; organic and inorganic pigments such as titanium dioxide; UV-absorbers; stabilisers; odor masking agents; viscosity enhancers; antioxidants such as, for example, ascorbyl palmitate, sodium bisulfite, sodium metabisulfite and the like, and mixtures thereof; preservatives such as, for example, potassium sorbate, sodium benzoate, sorbic acid, propyl gallate, benzylalcohol, methyl paraben, propyl paraben and the like; sequestering agents such as ethylene-diamine tetraacetic acid; flavoring agents such as natural vanillin; buffers such as citric acid and acetic acid; extenders or bulking agents such as silicates, diatomaceous earth, magnesium oxide or aluminum oxide; densification agents such as magnesium salts; and mixtures thereof. Additional ingredients may be included in order to control the duration of action of the biologically-active ingredient in the compositions and combined preparations of the invention. Control release compositions may thus be achieved by selecting appropriate polymer carriers such as for example polyesters, polyamino-acids, polyvinyl-pyrrolidone, ethylene-vinyl acetate copolymers, methylcellulose, carboxymethylcellulose, protamine sulfate and the like. The rate of drug release and duration of action may also be controlled by incorporating the active ingredient into particles, e.g. microcapsules, of a polymeric substance such as hydrogels, polylactic acid, hydroxymethyl-cellulose, polymethyl methacrylate and the other above-described polymers. Such methods include colloid drug delivery systems including, but not limited to liposomes, microspheres, microemulsions, nanoparticles, nanocapsules and so on. Depending on the route of administration, the pharmaceutical composition or combined preparation of the invention may also require protective coatings.

Pharmaceutical forms suitable for injectable use include sterile aqueous solutions or dispersions and sterile powders for the extemporaneous preparation thereof. Typical carriers for this purpose therefore include biocompatible aqueous buffers, ethanol, glycerol, propylene glycol, polyethylene glycol, complexing agents such as cyclodextrins and the like, and mixtures thereof.

Other modes of local drug administration can also be used. For example, the selected active agent may be administered by way of intracavernosal injection, or may be administered topically, in an ointment, gel or the like, or transdermal, including transscrotally, using a conventional transdermal drug delivery system. Intracavernosal injection can be carried out by use of a syringe or any other suitable device. An example of a hypodermic syringe useful herein is described in U.S. Pat. No. 4,127,118, injection being made on the dorsum of the penis by placement of the needle to the side of each dorsal vein and inserting it deep into the corpora.

A third aspect of the invention relates to the use of the compound of the first aspect of the invention or the pharmaceutical composition of the second aspect of the invention for the manufacture of a medicament for the prevention or treatment of a viral infection in a mammal.

A forth aspect of the invention relates to a method for prevention or treatment of a viral infection in a mammal, comprising providing to said subject a therapeutically effective amount of the compound of any one of the preceding claims.

A fifth aspect of the invention relates to a kit of parts comprising: (i) the compound or pharmaceutical composition according to any one of the preceding claims; and (ii) instructions for use.

A sixth aspect of the invention relates to a compound of the general formula (A), wherein in general formula (A) R is: (i) a straight or branched, saturated or unsaturated alkyl group; (ii) a cycloalkyl group; (iii) a straight or branched aryl group; (iv) an alkylaryl group; (v) an alkoxyaryl group; or (vi) an alkylaminoaryl group; and wherein the compound does not comprise the compounds having the formula below.

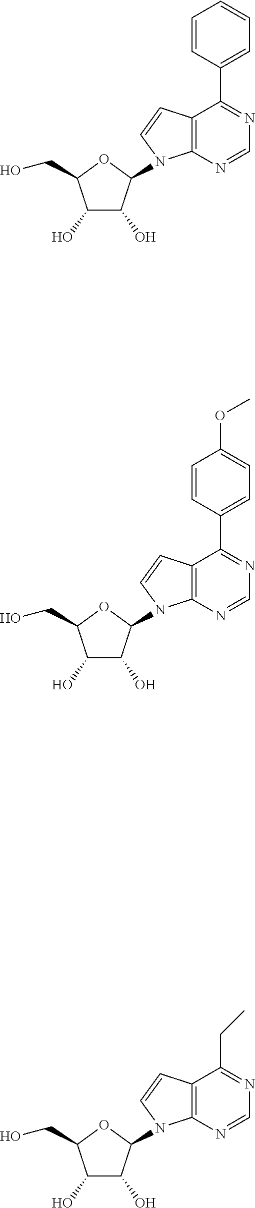 [SMILES: OC[C@H]1O[C@@H](n2ccc3c(-c4ccccc4)ncnc32)[C@H](O)[C@@H]1O ; COc1ccc(-c2ncnc3c2ccn3[C@@H]2O[C@H](CO)[C@@H](O)[C@H]2O)cc1 ; CCc1ncnc2c1ccn2[C@@H]1O[C@H](CO)[C@@H](O)[C@H]1O]

The compounds of the formula disclaimed above have been previously synthesized by Nauš et al. as disclosed above (Nauš, P. J. et al. Med. Chem. 2010, 53(1), 460-470), but their use as a medicament and/or their antiviral activity had not been characterized before.

A seventh aspect of the invention relates to a method for synthesizing purine modified nucleoside analogues comprising a cross-coupling reaction of aryl or alkyl Grignard reagents with halogenated purine nucleosides, wherein the catalyst in the cross-coupling reaction is: (i) iron; or (ii) an iron/copper mixture.

In some embodiments of the invention, the purine modified nucleoside analogue is a pyrrolopyrimidine modified nucleoside analogue.

The method of the invention is exemplified in detail through the examples of the present application.

The present invention will be further described with reference to certain more specific embodiments and examples, but the present invention is not limited thereto. The following examples are given by way of illustration only.

### Definitions

When describing the compounds of the invention, the terms used are to be construed in accordance with the following definitions, unless a context dictates otherwise.

“Alkyl” means a straight-chain or branched hydrocarbon chain with up to 6 carbon atoms. Each hydrogen of an alkyl carbon may be replaced by a substituent as further specified herein.

“Alkenyl” means a straight-chain or branched hydrocarbon chain that contains at least one carbon-carbon double bond. Each hydrogen of an alkenyl carbon may be replaced by a substituent as further specified herein.

“Alkynyl” means a straight-chain or branched hydrocarbon chain that contains at least one carbon-carbon triple bond. Each hydrogen of an alkynyl carbon may be replaced by a substituent as further specified herein.

“C1-3 alkyl” means an alkyl chain having 1-3 carbon atoms, e.g. if present at the end of a molecule: methyl, ethyl, n-propyl, isopropyl, or e.g. —CH2-, —CH2-CH2-, —CH(CH3)-, —CH2-CH2-CH2-, —CH(C2H5)-, —C(CH3)2-, when two moieties of a molecule are linked by the alkyl group. Each hydrogen of a C1-3 alkyl carbon may be replaced by a substituent as further specified herein.

“C1-4 alkyl” means an alkyl chain having 1-4 carbon atoms, e.g. if present at the end of a molecule: methyl, ethyl, n-propyl, isopropyl, n-butyl, isobutyl, sec-butyl, tert-butyl, or e.g. —CH2-, —CH2-CH2-, —CH(CH3)-, —CH2-CH2-CH2-, —CH(C2H5)-, —C(CH3)2-, when two moieties of a molecule are linked by the alkyl group. Each hydrogen of a C1-4 alkyl carbon may be replaced by a substituent as further specified herein.

“C1-6 alkyl” means an alkyl chain having 1-6 carbon atoms, e.g. if present at the end of a molecule: C1-4 alkyl, methyl, ethyl, n-propyl, isopropyl, n-butyl, isobutyl, sec-butyl; tert-butyl, n-pentyl, n-hexyl, or e.g. —CH2-, —CH2-CH2-, —CH(CH3)-, —CH2-CH2-CH2-, —CH(C2H5)-, —C(CH3)2-, when two moieties of a molecule are linked by the alkyl group. Each hydrogen of a C1-6 alkyl carbon may be replaced by a substituent as further specified herein. The term “C1-12 alkyl” is defined accordingly.

“C2-6 alkenyl” means an alkenyl chain having 2 to 6 carbon atoms, e.g. if present at the end of a molecule: —CH═CH2, —CH═CH—CH3, —CH2-CH═CH2, —CH═CH—CH2-CH3, —CH═CH—CH═CH2, or e.g. —CH═CH—, when two moieties of a molecule are linked by the alkenyl group.

Each hydrogen of a C2-6 alkenyl carbon may be replaced by a substituent as further specified herein. The term “C2-12 alkenyl” is defined accordingly.

“C2-6 alkynyl” means an alkynyl chain having 2 to 6 carbon atoms, e.g. if present at the end of a molecule: —C≡CH, —CH2-C≡CH, CH2-CH2-C≡CH, CH2-C≡C—CH3, or e.g. —C≡C— when two moieties of a molecule are linked by the alkynyl group. Each hydrogen of a C2-6 alkynyl carbon may be replaced by a substituent as further specified herein. The term “C2-12 alkynyl” is defined accordingly.

As used herein with respect to a substituting radical, and unless otherwise stated, the term “acyl” broadly refers to a substituent derived from an acid such as an organic monocarboxylic acid, a carbonic acid, a carbamic acid (resulting into a carbamoyl substituent) or the thioacid or imidic acid (resulting into a carbamidoyl substituent) corresponding to said acids, and the term “sulfonyl” refers to a substituent derived from an organic sulfonic acid, wherein said acids comprise an aliphatic, aromatic or heterocyclic group in the molecule.

Acyl and sulfonyl groups originating from aliphatic or cycloaliphatic monocarboxylic acids or sulfonic acids are designated herein as aliphatic or cycloaliphatic acyl and sulfonyl groups and include, but are not limited to, the following:
alkanoyl (for example formyl, acetyl, propionyl, butyryl, isobutyryl, valeryl, isovaleryl, pivaloyl and the like);
cycloalkanoyl (for example cyclobutanecarbonyl, cyclopentanecarbonyl, cyclohexanecarbonyl, 1-adamantanecarbonyl and the like);
cycloalkyl-alkanoyl (for example cyclohexylacetyl, cyclopentylacetyl and the like);
alkenoyl (for example acryloyl, methacryloyl, crotonoyl and the like);
alkylthioalkanoyl (for example methylthioacetyl, ethylthioacetyl and the like);
alkanesulfonyl (for example mesyl, ethanesulfonyl, propanesulfonyl and the like);
alkoxycarbonyl (for example methoxycarbonyl, ethoxycarbonyl, propoxycarbonyl, isopropoxycarbonyl, butoxycarbonyl, isobutoxycarbonyl and the like);
alkylcarbamoyl (for example methylcarbamoyl and the like);
(N-alkyl)-thiocarbamoyl (for example (N-methyl)-thiocarbamoyl and the like);
alkylcarbamidoyl (for example methylcarbamidoyl and the like); and
alkoxalyl (for example methoxalyl, ethoxalyl, propoxalyl and the like);

Acyl and sulfonyl groups may also originate from aromatic monocarboxylic acids and include, but are not limited to, the following:
aroyl (for example benzoyl, toluoyl, xyloyl, 1-naphthoyl, 2-naphthoyl and the like);
aralkanoyl (for example phenylacetyl and the like);
aralkenoyl (for example cinnamoyl and the like);
aryloxyalkanoyl (for example phenoxyacetyl and the like);
arylthioalkanoyl (for example phenylthioacetyl and the like);
arylaminoalkanoyl (for example N-phenylglycyl, and the like);
arylsulfonyl (for example benzenesulfonyl, toluenesulfonyl, naphthalene sulfonyl and the like);
aryloxycarbonyl (for example phenoxycarbonyl, naphthyloxycarbonyl and the like);
aralkoxycarbonyl (for example benzyloxycarbonyl and the like);
arylcarbamoyl (for example phenylcarbamoyl, naphthylcarbamoyl and the like);
arylglyoxyloyl (for example phenylglyoxyloyl and the like).
arylthiocarbamoyl (for example phenylthiocarbamoyl and the like); and
arylcarbamidoyl (for example phenylcarbamidoyl and the like).

Acyl groups may also originate from an heterocyclic monocarboxylic acids and include, but are not limited to, the following:
heterocyclic-carbonyl, in which said heterocyclic group is as defined herein, preferably an aromatic or non-aromatic 5- to 7-membered heterocyclic ring with one or more heteroatoms selected from the group consisting of nitrogen, oxygen and sulfur in said ring (for example thiophenoyl, furoyl, pyrrolecarbonyl, nicotinoyl and the like); and
heterocyclic-alkanoyl in which said heterocyclic group is as defined herein, preferably an aromatic or non-aromatic 5- to 7-membered heterocyclic ring with one or more heteroatoms selected from the group consisting of nitrogen, oxygen and sulfur in said ring (for example thiopheneneacetyl, furylacetyl, imidazolylpropionyl, tetrazolylacetyl, 2-(2-amino-4-thiazolyl)-2-methoxyiminoacetyl and the like).

As used herein with respect to a substituting radical, and unless otherwise stated, the term “cycloalkyl” means a mono- or polycyclic saturated hydrocarbon monovalent radical, such as for instance cyclopropyl, cyclobutyl, cyclopentyl, cyclohexyl, cycloheptyl, cyclooctyl and the like, or a C₇₋₁₀ polycyclic saturated hydrocarbon monovalent radical having from 7 to 10 carbon atoms such as, for instance, norbornyl, fenchyl, trimethyltricycloheptyl or adamantyl. By way of example, “C3-7 cycloalkyl” or “C3-7 cycloalkyl ring” means a cyclic alkyl chain having 3-7 carbon atoms, e.g. cyclopropyl, cyclobutyl, cyclopentyl, cyclohexyl, cyclohexenyl, cycloheptyl. Preferably, cycloalkyl refers to cyclopropyl, cyclobutyl, cyclopentyl, cyclohexyl, or cycloheptyl. Each hydrogen of a cycloalkyl carbon may be replaced by a substituent as specified herein.

As used herein with respect to a substituting radical, and unless otherwise stated, the term “aryl” designate any mono- or polycyclic aromatic monovalent hydrocarbon radical having from 6 up to 30 carbon atoms such as but not limited to phenyl, naphthyl, anthracenyl, phenantracyl, fluoranthenyl, chrysenyl, pyrenyl, biphenylyl, terphenyl, picenyl, indenyl, biphenyl, indacenyl, benzocyclobutenyl, benzocyclooctenyl and the like, including fused benzo-C₄-β cycloalkyl radicals (the latter being as defined above) such as, for instance, indanyl, tetrahydronaphthyl, fluorenyl and the like, all of the said radicals being optionally substituted with one or more substituents independently selected from the group consisting of halogen, amino, trifluoromethyl, hydroxyl, sulfhydryl and nitro, such as for instance 4-fluorophenyl, 4-chlorophenyl, 3,4-dichlorophenyl, 4-cyanophenyl, 2,6-dichlorophenyl, 2-fluorophenyl, 3-chlorophenyl, 3,5-dichlorophenyl and the like.

As used herein, e.g. with respect to a substituting radical such as the combination of substituents in certain positions of the compounds, and unless otherwise stated, the term “homocyclic” means a mono- or polycyclic, saturated or mono-unsaturated or polyunsaturated hydrocarbon radical having from 4 up to 15 carbon atoms but including no heteroatom in the said ring; for instance said combination of substituents may form a C2-s alkylene radical, such as tetramethylene, which cyclizes with the carbon atoms in certain positions of the thiazolo[5,4-d]pyrimidine, oxazolo[5,4-d]pyrimidine, thieno[2,3-d]pyrimidine or purine ring.

As used herein, e.g. with respect to a substituting radical such as the combination of substituents in certain positions of the compounds, and unless otherwise stated, the term “heterocyclic” means a mono- or polycyclic, saturated or mono-unsaturated or polyunsaturated monovalent hydrocarbon radical having from 2 up to 15 carbon atoms and including one or more heteroatoms in one or more heterocyclic rings, each of said rings having from 3 to 10 atoms (and optionally further including one or more heteroatoms attached to one or more carbon atoms of said ring, for instance in the form of a carbonyl or thiocarbonyl or selenocarbonyl group, and/or to one or more heteroatoms of said ring, for instance in the form of a sulfone, sulfoxide, N-oxide, phosphate, phosphonate or selenium oxide group), each of said heteroatoms being independently selected from the group consisting of nitrogen, oxygen, sulfur, selenium and phosphorus, also including radicals wherein a heterocyclic ring is fused to one or more aromatic hydrocarbon rings for instance in the form of benzo-fused, dibenzo-fused and naphtho-fused heterocyclic radicals; within this definition are included heterocyclic radicals such as, but not limited to, diazepinyl, oxadiazinyl, thiadiazinyl, dithiazinyl, triazolonyl, diazepinonyl, triazepinyl, triazepinonyl, tetrazepinonyl, benzoquinolinyl, benzothiazinyl, benzothiazinonyl, benzoxa-thiinyl, benzodioxinyl, benzodithiinyl, benzoxazepinyl, benzothiazepinyl, benzodiazepine, benzodioxepinyl, benzodithiepinyl, berrzoxazocinyl, benzo-thiazocinyl, benzodiazocinyl, benzoxathiocinyl, benzodioxocinyl, benzotrioxepinyl, benzoxathiazepinyl, benzoxadiazepinyl, benzothia-diazepinyl, benzotriazepinyl, benzoxathiepinyl, benzotriazinonyl, benzoxazolinonyl, azetidinonyl, azaspiroundecyl, dithiaspirodecyl, selenazinyl, selenazolyl, selenophenyl, hypoxanthinyl, azahypo-xanthinyl, bipyrazinyl, bipyridinyl, oxazolidinyl, diselenopyrimidinyl, benzodioxocinyl, benzopyrenyl, benzopyranonyl, benzophenazinyl, benzoquinolizinyl, dibenzo-carbazolyl, dibenzoacridinyl, dibenzophenazinyl, dibenzothiepinyl, dibenzoxepinyl, dibenzopyranonyl, dibenzoquinoxalinyl, dibenzothiazepinyl, dibenzisoquinolinyl, tetraazaadamantyl, thiatetraazaadamantyl, oxauracil, oxazinyl, dibenzothiophenyl, dibenzofuranyl, oxazolinyl, oxazolonyl, azaindolyl, azolonyl, thiazolinyl, thiazolonyl, thiazolidinyl, thiazanyl, pyrimidonyl, thiopyrimidonyl, thiamorpholinyl, azlactonyl, naphtindazolyl, naphtindolyl, naphtothiazolyl, naphtothioxolyl, naphtoxindolyl, naphto-triazolyl, naphtopyranyl, oxabicycloheptyl, azabenzimidazolyl, azacycloheptyl, azacyclooctyl, azacyclononyl, azabicyclononyl, tetrahydrofuryl, tetrahydropyrarryl, tetrahydro-pyronyl, tetrahydroquinoleinyl, tetrahydrothienyl and dioxide thereof, dihydrothienyl dioxide, dioxindolyl, dioxinyl, dioxenyl, dioxazinyl, thioxanyl, thioxolyl, thiourazolyl, thiotriazolyl, thiopyranyl, thiopyronyl, coumarinyl, quinoleinyl, oxyquinoleinyl, quinuclidinyl, xanthinyl, dihydropyranyl, benzodihydrofuryl, benzothiopyronyl, benzothiopyranyl, benzoxazinyl, benzoxazolyl, benzodioxolyl, benzodioxanyl, benzothiadiazolyl, benzotriazinyl, benzothiazolyl, benzoxazolyl, phenothioxinyl, phenothiazolyl, phenothienyl (benzothiofuranyl), phenopyronyl, phenoxazolyl, pyridinyl, dihydropyridinyl, tetrahydropyridinyl, piperidinyl, morpholinyl, thiomorpholinyl, pyrazinyl, pyrimidinyl, pyridazinyl, triazinyl, tetrazinyl, triazolyl, benzotriazolyl, tetrazolyl, imidazolyl, pyrazolyl, thiazolyl, thiadiazolyl, isothiazolyl, oxazolyl, oxadiazolyl, pyrrolyl, furyl, dihydrofutyl, furoyl, hydantoinyl, dioxolanyl, dioxolyl, dithianyl, dithienyl, dithiinyl, thienyl, indolyl, indazolyl, benzofutyl, quinolyl, quinazolinyl, quinoxalinyl, carbazolyl, phenoxazinyl, phenothiazinyl, xanthenyl, purinyl, benzothienyl, naphtothienyl, thianthrenyl, pyranyl, pyronyl, benzopyronyl, isobenzofuranyl, chromenyl, phenoxathiinyl, indolizinyl, quinolizinyl, isoquinolyl, phthalazirryl, naphthiridinyl, cinnolinyl, pteridinyl, carbolinyl, acridinyl, perimidinyl, phenanthrolinyl, phenazinyl, phenothiazinyl, imidazolinyl, imidazolidinyl, benzimidazolyl, pyrazolinyl, pyrazolidinyl, pyrrolinyl, pyrrolidinyl, piperazinyl, uridinyl, thymidinyl, cytidinyl, azirinyl, aziridinyl, diazirinyl, diaziridinyl, oxiranyl, oxaziridinyl, dioxiranyl, thiiranyl, azetyl, dihydroazetyl, azetidinyl, oxetyl, oxetanyl, oxetanonyl, homopiperazinyl, homopiperidinyl, thietyl, thietanyl, diazabicyclooctyl, diazetyl, diaziridinonyl, diaziridinethionyl, chromanyl, chromanonyl, thiochromanyl, thiochromanonyl, thiochromenyl, benzofuranyl, benzisothiazolyl, benzocarbazolyl, benzochromonyl, benzisoalloxazinyl, benzocoumarinyl, thiocoumarinyl, pheno-metoxazinyl, phenoparoxazinyl, phentriazinyl, thiodiazinyl, thiodiazolyl, indoxyl, thioindoxyl, benzodiazinyl (e.g. phtalazinyl), phtalidyl, phtalimidinyl, phtalazonyl, alloxazinyl, dibenzopyronyl (i.e. xanthonyl), xanthionyl, isatyl, isopyrazolyl, isopyrazolonyl, urazolyl, urazinyl, uretinyl, uretidinyl, succinyl, succinimido, benzylsultimyl, benzylsultamyl and the like, including all possible isomeric forms thereof, wherein each carbon atom of said heterocyclic ring may furthermore be independently substituted with a substituent selected from the group consisting of halogen, nitro, C₁₋₇ alkyl (optionally containing one or more functions or radicals selected from the group consisting of carbonyl (oxo), alcohol (hydroxyl), ether (alkoxy), acetal, amino, imino, oximino, alkyloximino, amino-acid, cyano, carboxylic acid ester or amide, nitro, thio C₁₋₇ alkyl, thio C₃₋₁₀ cycloalkyl, C₁₋₇ alkylamino, cycloalkylamino, alkenylamino, cycloalkenylamino, alkynylamino, arylamino, arylalkyl−amino, hydroxylalkylamino, mercaptoalkylamino, heterocyclic-substituted alkylamino, heterocyclic amino, heterocyclic-substituted arylamino, hydrazino, alkylhydrazino, phenylhydrazino, sulfonyl, sulfonamido and halogen), C₃₋₇ alkenyl, C₂₋₇ alkynyl, halo C₁₋₇ alkyl, C₃₋₁₀ cycloalkyl, aryl, arylalkyl, alkylaryl, alkylacyl, arylacyl, hydroxyl, amino, C₁₋₇ alkylamino, cycloalkylamino, alkenylamino, cycloalkenylamino, alkynylamino, arylamino, arylalkylamino, hydroxyalkylamino, mercaptoalkylamino, heterocyclic-substituted alkylamino, heterocyclic amino, heterocyclic-substituted arylamino, hydrazino, alkylhydrazino, phenylhydrazino, sulfhydryl, C₁₋₇ alkoxy, C₃₋₁₀ cycloalkoxy, aryloxy, arylalkyloxy, oxyheterocyclic, heterocyclic-substituted alkyloxy, thio C₁₋₇ alkyl, thio C₃₋₁₀ cycloalkyl, thioaryl, thioheterocyclic, arylalkylthio, heterocyclic-substituted alkylthio, formyl, hydroxylamino, cyano, carboxylic acid or esters or thioesters or amides thereof, tricarboxylic acid or esters or thioesters or amides thereof; depending upon the number of unsaturations in the 3 to 10 atoms ring, heterocyclic radicals may be sub-divided into heteroaromatic (or “heteroaryl”) radicals and non-aromatic heterocyclic radicals; when a heteroatom of said non-aromatic heterocyclic radical is nitrogen, the latter may be substituted with a substituent selected from the group consisting of C₁₋₇ alkyl, C₃₋₁₀ cycloalkyl, aryl, arylalkyl and alkylaryl.

As used herein with respect to a substituting radical, and unless otherwise stated, the terms “arylalkyl”, “arylalkenyl” and “heterocyclic-substituted alkyl” refer to an aliphatic saturated or ethylenically unsaturated hydrocarbon monovalent radical (preferably a C₁₋₇ alkyl or C₂₋₇ alkenyl radical such as defined above) onto which an aryl or heterocyclic radical (such as defined above) is already bonded via a carbon atom, and wherein the said aliphatic radical and/or the said aryl or heterocyclic radical may be optionally substituted with one or more substituents independently selected from the group consisting of halogen, amino, hydroxyl, sulfhydryl, C₁₋₇ alkyl, C₁₋₇ alkoxy, trifluoromethyl and nitro, such as but not limited to benzyl, 4-chlorobenzyl, 4-fluorobenzyl, 2-fluorobenzyl, 3,4-dichlorobenzyl, 2,6- dichlorobenzyl, 3-methylbenzyl, 4-methylbenzyl, 4-ter-butylbenzyl, phenylpropyl, 1-naphthylmethyl, phenylethyl, 1-amino-2-phenylethyl, 1-amino-2-[4-hydroxy-phenyl]ethyl, 1-amino-2-[indol-2-yl]ethyl, styryl, pyridylmethyl (including all isomers thereof), pyridylethyl, 2-(2-pyridyl)isopropyl, oxazolylbutyl, 2-thienylmethyl, pyrrolylethyl, morpholinylethyl, imidazol-1-yl-ethyl, benzodioxolylmethyl and 2-furylmethyl.

As used herein with respect to a substituting radical, and unless otherwise stated, the terms “alkylaryl” and “alkyl-substituted heterocyclic” refer to an aryl or, respectively, heterocyclic radical (such as defined above) onto which are bonded one or more aliphatic saturated or unsaturated hydrocarbon monovalent radicals, preferably one or more C₁₋₆ alkyl, C₂₋₇ alkenyl or C₃₋₁₀ cycloalkyl radicals as defined above such as, but not limited to, o-toluyl, m-toluyl, p-toluyl, 2,3-xylyl, 2,4-xylyl, 3,4- xylyl, o-cumenyl, m-cumenyl, p-cumenyl, o-cymenyl, m-cymenyl, p-cymenyl, mesityl, ter-butylphenyl, lutidinyl (i.e. dimethylpyridyl), 2-methylaziridinyl, methyl−benzimidazolyl, methylbenzofuranyl, methylbenzothiazolyl, methylbenzotriazolyl, methylbenzoxazolyl and methylbenzselenazolyl.

As used herein with respect to a substituting radical, and unless otherwise stated, the term “alkoxyaryl” refers to an aryl radical (such as defined above) onto which is (are) bonded one or more C₁₋₇ alkoxy radicals as defined above, preferably one or more methoxy radicals, such as, but not limited to, 2-methoxyphenyl, 3-methoxyphenyl, 4-methoxyphenyl, 3,4-dimethoxyphenyl, 2,4,6-trimethoxyphenyl, methoxynaphtyl and the like.

As used herein with respect to a substituting radical, and unless otherwise stated, the terms “alkylamino”, “cycloalkylamino”, ‘.’ alkenylamino”, “cyclo-alkenylamino”, “arylamino”, “arylalkylamino”, “heterocyclic-substituted alkylamino”, “heterocyclic-substituted arylamino”, “heterocyclic amino”, “hydroxy-alkylamino”, “mercaptoalkylamino” and “alkynylamino” mean that respectively one (thus monosubstituted amino) or even two (thus disubstituted amino) C₁₋₇ alkyl, C₃₋₁₀ cycloalkyl, C₂₋₇ alkenyl, C₃₋₁₀ cycloalkenyl, aryl, arylalkyl, heterocyclic-substituted alkyl, heterocyclic-substituted aryl, heterocyclic (provided in this case the nitrogen atom is attached to a carbon atom of the heterocyclic ring), mono- or polyhydroxy C₁₋₇ alkyl, mono- or polymercapto C₁₋₇ alkyl, or C₂₋₇ alkynyl radical(s) (each of them as defined herein, respectively, and including the presence of optional substituents independently selected from the group consisting of halogen, amino, hydroxyl, sulfhydryl, C₁₋₇ alkyl, C₁₋₇ alkoxy, trifluoromethyl and nitro) is/are attached to a nitrogen atom through a single bond such as, but not limited to, anilino, 2- bromoanilino, 4-bromoanilino, 2-chloroanilino, 3-chloroanilino, 4-chloroanilino, 3- chloro-4-methoxyanilino, 5-chloro-2-methoxyanilino, 2,3-dimethylanilino, 2,4-dimethylanilino, 2,5-dimethylanilino, 2,6-dimethylanilino, 3,4-dimethylanilino, 2- fluoroanilino, 3-fluoroanilino, 4-fluoroanilino, 3-fluoro-2-methoxyanilino, 3-fluoro-4-methoxyanilino, 2-fluoro-4-methylanilino, 2-fluoro-5-methylanilino, 3-fluoro-2- methylanilino, 3-fluoro-4-methylanilino, 4-fluoro-2-methylanilino, 5-fluoro-2- methylanilino, 2-iodoanilino, 3-iodoanilino, 4-iodoanilino, 2-methoxy-5-methylanilino, 4-methoxy-2-methylanilino, 5-methoxy-2-methylanilino, 2-ethoxyanilino, 3-ethoxy-anilino, 4-ethoxyanilino, benzylamino, 2-methoxybenzylamino, 3-methoxybenzylamino, 4-methoxybenzylamino, 2-fluorobenzylamino, 3-fluorobenzylamino, 4-fluoro-benzylamino, 2-chlorobenzylamino, 3-chlorobenzylamino, 4-chlorobenzylamino, 2-aminobenzylamino, diphenylmethylamino, α-naphthylamino, methylamino, dimethylamino, ethylamino, diethylamino, isopropylamino, propenylamino, n-butylamino, ter-butylamino, dibutylamino, 1,2-diaminopropyl, 1,3-diaminopropyl, 1,4-diaminobutyl, 1,5-diaminopentyl, 1,6-diaminohexyl, morpholinomethylamino, 4-morpholinoanilino, hydroxymethylamino, β-hydroxyethylamino and ethynylamino; this definition also includes mixed disubstituted amino radicals wherein the nitrogen atom is attached to two such radicals belonging to two different sub-sets of radicals, e.g. an alkyl radical and an alkenyl radical, or to two different radicals within the same subset of radicals, e.g. methylethylamino; among di-substituted amino radicals, symmetrically-substituted amino radicals are more easily accessible and thus usually preferred from a standpoint of ease of preparation.

As used herein and unless otherwise stated, the term “enantiomer” means each individual optically active form of a compound of the invention, having an optical purity or enantiomeric excess (as determined by methods standard in the art) of at least 80% (i.e. at least 90% of one enantiomer and at most 10% of the other enantiomer), preferably at least 90% and more preferably at least 98%.

As used herein and unless otherwise stated, the term “solvate” includes any combination which may be formed by a thiazolo[5,4-d]pyrimidine, oxazolo[5,4-d]pyrimidine, thieno[2,3-d]pyrimidine or purine derivative of this invention with a suitable inorganic solvent (e.g. hydrates) or organic solvent, such as but not limited to alcohols, ketones, esters, ethers, nitriles and the like.

The following examples illustrate the present invention.

### EXAMPLES

### Materials and Methods

General Information. All reagents and solvents were purchased from commercial sources and used as obtained. Moisture sensitive reactions were carried out using oven-dried glassware under a nitrogen or argon atmosphere. 1H NMR and 13C NMR spectra were recorded on a Bruker Avance 300 MHz spectrometer using tetramethylsilane as internal standard or referenced to the residual solvent signal. The following abbreviations were used to indicate multiplicities: s (singlet), d (doublet), t (triplet), q (quartet), m (multiplet), br (broad), and dd (doublet of doublets). Coupling constants are expressed in hertz (Hz). High-resolution mass spectra (HRMS) were obtained on a quadrupole orthogonal acceleration time-of-flight mass spectrometer (Synapt G2 HDMS, Waters, Milford, Mass.). Samples were infused at 3 μL/min and spectra were obtained in positive ionization mode with a resolution of 15000 (fwhm) using leucine enkephalin as lock mass. Pre-coated aluminum sheets (254 nm) were used for thin layer chromatography (TLC), and spots were visualized with UV light. All products were purified by flash column chromatography on silica gel (40-60μ, 60 Å).

General Procedure for the FeCl3 Catalyzed Cross-Coupling of 6-Chloro-7-Deazapurine with Grignard Reagents. An oven-dried flask was charged with 6-chloro-7-deazapurine (1.3 mmol, 1 equiv) in THF 5 mL, NMP 0.5 mL, FeCl3 (0.13 mmol, 0.1 equiv). The mixture was cooled to 0° C., and a solution of RMgX (2.0-8.0 mmol, 1.5-6.2 equiv) in THF was added. The reaction mixture was stirred for 2 h with gradual warming to room temperature. Monitoring with TLC till starting material disappeared, the reaction was quenched by the addition of aq. saturated solution of NH4Cl and extracted with EtOAc (3×10 mL). The organic solution was dried over Na2SO4 and concentrated under reduced pressure. The crude product was purified by silica gel chromatography to afford the desired product.

Example 1. Synthesis of 6-Phenyl-7-deazapurine (5a). Following the general procedure, compound 5a was obtained starting from 6-chloro-7-deazapurine (4) (200 mg, 1.3 mmol), FeCl3 (21 mg, 1.3 mmol), phenylmagnesium bromide 1 M in THF (539.82 mg, 3.0 mmol, added as portions and TLC monitor) in dry THF (5.0 mL) and NMP (0.5 mL) after column chromatography on silica gel (Heptane/EtOAc=5:1, to DCM:MeOH=30:1, v/v) as white solid (166 mg, 65% yield). 1H NMR (300 MHz, DMSO-d6) δ 12.27 (br, 1H, NH), 8.85 (s, 1H, H-2), 8.17 (m, 2H, Ph-H), 7.65 (d, J8,7=3.6 Hz, 1H, H-8), 7.62-7.52 (m, 3H, Ph-H), 6.88 (d, J7,8=3.6 Hz, 1H, H-7); 13C{1H} NMR (75 MHz, DMSO-d6) δ 155.7 (C-6), 152.7 (C-4), 151.0 (C-2), 138.0 (C-Ph), 130.0 (C-Ph), 128.9 (C-Ph), 128.6 (C-Ph), 127.7 (C-8), 114.6 (C-5), 100.0 (C-7); HRMS (ESI-TOF) m/z: calcd for C12H9N3 ([M+H]+), 196.0869, found 196.0871.

Example 2. Synthesis of 6-(4-Methoxylphenyl)-7-deazapurine (5b). Following the general procedure, compound 5b was obtained starting from 6-chloro-7-deazapurine (4) (200 mg, 1.3 mmol), FeCl3 (21 mg, 1.3 mmol), 4-methoxylphenylmagnesium bromide 1 M in THF (545.87 mg, 2.6 mmol, added as portions and TLC monitor) in dry THF (5.0 mL) and NMP (0.5 mL) after column chromatography on silica gel (Heptane/EtOAc=5:1, to DCM:MeOH=30:1, v/v) as white solid (206 mg, 70% yield). 1H NMR (300 MHz, DMSO-d6) δ 12.20 (br, 1H, NH), 8.79 (s, 1H, H-2), 8.18 (d, J=9.1 Hz, 2H, Ph-H), 7.61 (d, J8,7=3.6 Hz, 1H, H-8), 7.12 (d, J=9.1 Hz, 2H, Ph-H), 6.87 (d, J7, 8=3.6 Hz, 1H, H-7), 3.41 (s, 3H, OCH3); 13C{1H} NMR (75 MHz, DMSO-d6) δ 160.9 (C-6), 155.3 (C-4), 152.6 (C-Ph), 150.9 (C-2), 130.5 (C-Ph), 130.2 (C-Ph), 127.3 (C-8), 114.3 (C-5), 113.9 (C-Ph), 100.1 (C-7), 55.4 (OCH3); HRMS (ESI-TOF) m/z: calcd for C13H11N3O ([M+H]+), 226.0974, found 226.0979.

Example 3. Synthesis of 6-(4-Methylphenyl)-7-deazapurine (5c). Following the general procedure, compound 5c was obtained starting from 6-chloro-7-deazapurine (4) (200 mg, 1.3 mmol), FeCl3 (21 mg, 1.3 mmol), 4-methylphenylmagnesium bromide 1 M in THF (775.84 mg, 4.0 mmol, added as portions added as portions and TLC monitor) in dry THF (5.0 mL) and NMP (0.5 mL) after column chromatography on silica gel (Heptane/EtOAc=5:1, to DCM:MeOH=30:1, v/v) as white solid (185 mg, 68% yield). 1H NMR (300 MHz, DMSO-d6) δ 12.23 (br, 1H, NH), 8.81 (s, 1H, H-2), 8.08 (d, J=8.0 Hz, 2H, Ph-H), 7.63 (dd, J8,7=3.6 Hz, J8,NH=2.4 Hz, 1H, H-8), 7.38 (d, J=7.9 Hz, 2H, Ph-H), 6.87 (d, J7,8=3.6 Hz, 1H, H-7), 2.39 (s, 3H, CH3); 13C{1H} NMR (75 MHz, DMSO-d6) δ 155.7 (C-6), 152.7 (C-4), 151.0 (C-2), 139.8 (C-Ph), 135.3 (C-Ph), 129.5 (C-Ph), 128.6 (C-Ph), 127.5 (C-8), 125.8 (C-Ph), 114.2 (C-5), 100.1 (C-7), 21.0 (CH3); HRMS (ESI-TOF) m/z: calcd for C13H11N3 ([M+H]+), 210.1025, found 210.1029.

Example 4. Synthesis of 6-(4-Ethylphenyl)-7-deazapurine (5d). Following the general procedure, compound 5d was obtained starting from 6-chloro-7-deazapurine (4) (200 mg, 1.3 mmol), FeCl3 (21 mg, 1.3 mmol), 4-ethylphenylmagnesium bromide 1 M in THF (2.72 g, 13.0 mmol, added as portions and TLC monitor) in dry THF (5.0 mL) and NMP (0.5 mL) after column chromatography on silica gel (Heptane/EtOAc=5:1, to DCM:MeOH=30:1, v/v) as white solid (145 mg, 50% yield). 1H NMR (300 MHz, DMSO-d6) δ 12.24 (br, 1H, NH), 8.82 (s, 1H, H-2), 8.12 (d, J=8.1 Hz, 2H, Ph-H), 7.63 (dd, J8,7=3.6 Hz, J8,NH=2.4 Hz, 1H, H-8), 7.41 (d, J=8.0 Hz, 2H, Ph-H), 6.87 (dd, J7,8=3.6 Hz, J7,NH=1.8 Hz, 1H, H-7), 2.69 (q, J=7.4 Hz, 2H, CH2CH3), 1.23 (t, J=7.5 Hz, 2H, CH2CH3); 13C{1H} NMR (75 MHz, DMSO-d6) δ 155.7 (C-6), 152.7 (C-4), 151.0 (C-2), 146.0 (C-Ph), 128.6 (C-Ph), 128.3 (C-Ph), 127.5 (C-8), 114.4 (C-5), 100.1 (C-7), 28.1 (CH2CH3), 15.4 (CH2CH3); HRMS (ESI-TOF) m/z: calcd for C14H13N3 ([M+H]+), 224.1182, found 224.1180.

Example 5. Synthesis of 6-Cyclopropyl-7-deazapurine (5e). Following the general procedure, compound 5e was obtained starting from 6-chloro-7-deazapurine (4) (200 mg, 1.3 mmol), FeCl3 (21 mg, 1.3 mmol), 4-cyclopropylmagnesium bromide 0.7 M in THF (244.07 mg, 1.7 mmol, added as portions and TLC monitor) in dry THF (5.0 mL) and NMP (0.5 mL) after column chromatography on silica gel (Heptane/EtOAc=5:1, to DCM:MeOH=30:1, v/v) as white solid (154 mg, 74% yield). 1H NMR (300 MHz, CD3OD) δ 8.50 (s, 1H, H-2), 7.38 (d, J8,7=3.6 Hz, H-8), 6.74 (d, J7,8=3.6 Hz, H-7), 2.51-2.42 (m, 1H, CH(CH2)2), 1.32-1.41 (m, 4H, CH(CH2)2); 13C{1H} NMR (75 MHz, CD3OD) δ 163.9 (C-6), 149.9 (C-2), 149.6 (C-4), 124.9 (C-8), 116.6 (C-5), 98.6 (C-7), 13.6 (CH(CH2)2), 9.3 (2×CH(CH2)2); HRMS (ESI-TOF) m/z: calcd for C9H9N3 ([M+H]+), 160.0869, found 160.0871.

Example 6. Synthesis of 6-Isopropyl-7-deazapurine (5f). Following the general procedure, compound 5f was obtained starting from 6-chloro-7-deazapurine (4) (200 mg, 1.3 mmol), FeCl3 (21 mg, 1.3 mmol), 4-isopropylmagnesium bromide 3 M in THF (662.84 mg, 4.5 mmol, added as portions and TLC monitor) in dry THF (5.0 mL) and NMP (0.5 mL) after column chromatography on silica gel (Heptane/EtOAc=5:1, to DCM:MeOH=30:1, v/v) as white solid (180 mg, 86% yield). 1H NMR (300 MHz, CD3OD) δ 8.64 (s, 1H, H-2), 7.41 (d, J8,7=3.6 Hz, H-8), 6.66 (d, J7,8=3.6 Hz, H-7), 3.52-3.43 (m, 1H, CH(CH3)2), 1.41 (d, J=6.9 Hz, 6H, CH(CH3)2); 13C{1H} NMR (75 MHz, CD3OD) δ 167.1 (C-6), 150.8 (C-2), 149.8 (C-4), 125.3 (C-8), 115.6 (C-5), 98.8 (C-7), 33.3 (CH(CH3)2), 20.0 (CH(CH3)2); HRMS (ESI-TOF) m/z: calcd for C9H11N3 ([M+H]+), 162.1025, found 162.1027.

Example 7. Synthesis of 6-Methyl-7-deazapurine (5g). Following the general procedure, compound 5g was obtained starting from 6-chloro-7-deazapurine (4) (200 mg, 1.3 mmol), FeCl3 (21 mg, 1.3 mmol), methylmagnesium bromide 3 M in THF (536.60 mg, 4.5 mmol, added as portions and TLC monitor) in dry THF (5.0 mL) and NMP (0.5 mL) after column chromatography on silica gel (Heptane/EtOAc=5:1, to DCM:MeOH=30:1, v/v) as white solid (133 mg, 77% yield). 1H NMR (300 MHz, CD3OD) δ 8.58 (s, 1H, H-2), 7.41 (d, J8,7=3.6 Hz, 1H, H-8), 6.64 (d, J7,8=3.6 Hz, 1H, H-7), 2.70 (s, 3H, CH3); 13C{1H} NMR (75 MHz, CD3OD) δ 158.4 (C-6), 150.2 (C-2), 149.4 (C-4), 125.4 (C-8), 117.3 (C-5), 99.0 (C-7), 19.2 (CH3); HRMS (ESI-TOF) m/z: calcd for C7H7N3 ([M+H]+), 134.0712, found 134.0710.

Example 8. Synthesis of 6-Cyclohexyl-7-deazapurine (5h). Following the general procedure, compound 5h was obtained starting from 6-chloro-7-deazapurine (4) (200 mg, 1.3 mmol), FeCl3 (21 mg, 1.3 mmol), cyclohexyl magnesium bromide 0.5 M in THF (309.15 mg, 1.7 mmol, added as portions and TLC monitor) in dry THF (5.0 mL) and NMP (0.5 mL) after column chromatography on silica gel (Heptane/EtOAc=5:1, to DCM:MeOH=30:1, v/v) as white solid (190 mg, 73% yield). 1H NMR (300 MHz, CDCl3) δ 11.49 (br, 1H, NH), 8.86 (s, 1H, H-2), 7.35 (dd, J8,7=3.6 Hz, J8,NH=2.4 Hz, 1H, H-8), 6.66 (dd, J7,8=3.6 Hz, J7,NH=1.8 Hz, 1H, H-7), 3.18-3.06 (m, 1H, CH(CH2)5), 2.00-1.39 (m, 10H, CH(CH2)5); 13C{1H}NMR (75 MHz, CDCl3) δ 167.6 (C-6), 152.0 (C-2), 151.4 (C-4), 124.7 (C-8), 116.6 (C-5), 100.1 (C-7), 44.6 (CH(CH2)5), 31.9, 26.7, 26.7, 26.3, 26.3 (5×CH2); HRMS (ESI-TOF) m/z: calcd for C12H15N3 ([M+H]+), 202.1338, found 202.1338.

Example 9. Synthesis of 6-Ethyl-7-deazapurine (5i). Following the general procedure, compound 5i was obtained starting from 6-chloro-7-deazapurine (4) (200 mg, 1.3 mmol), FeCl3 (21 mg, 1.3 mmol), ethylmagnesium bromide 2 M in THF (266.54 mg, 2.0 mmol, added as portions and TLC monitor) in dry THF (5.0 mL) and NMP (0.5 mL) after column chromatography on silica gel (Heptane/EtOAc=5:1, to DCM:MeOH=30:1, v/v) as white solid (164 mg, 85% yield). 1H NMR (300 MHz, CD3OD) δ 8.62 (s, 1H, H-2), 7.41 (d, J8,7=3.6 Hz, 1H, H-8), 6.66 (d, J7,8=3.6 Hz, 1H, H-7), 3.04 (q, J=7.6 Hz, 2H, CH2), 1.37 (t, J=7.6 Hz, 3H, CH3); 13C{1H} NMR (75 MHz, CD3OD) δ 163.4 (C-6), 150.6 (C-2), 149.7 (C-4), 125.4 (C-8), 116.4 (C-5), 98.8 (C-7), 27.4 (CH2), 11.7 (CH3); HRMS (ESI-TOF) m/z: calcd for C8H9N3 ([M+H]+), 148.0869, found 148.0874.

Example 10. Synthesis of 6-Cyclopentyl-7-deazapurine (5j). Following the general procedure, compound 5j was obtained starting from 6-chloro-7-deazapurine (4) (200 mg, 1.3 mmol), FeCl3 (21 mg, 1.3 mmol), cyclopentylmagnesium bromide 0.5 M in THF (286.0 mg, 1.7 mmol, added as portions and TLC monitor) in dry THF (5.0 mL) and NMP (0.5 mL) after column chromatography on silica gel (Heptane/EtOAc=5:1, to DCM:MeOH=30:1, v/v) as white solid (190 mg, 75% yield). 1H NMR (300 MHz, CDCl3) δ 11.07 (br, 1H, NH), 8.85 (s, 1H, H-2), 7.33 (dd, J8,7=3.6 Hz, J8,NH=2.4 Hz, 1H, H-8), 6.64 (dd, J7,8=3.6 Hz, J7,NH=1.8 Hz, 1H, H-7), 3.36-3.53 (m, 1H, CH(CH2)4), 2.17-1.75 (m, 8H, CH(CH2)4); 13C{1H}NMR (75 MHz, CDCl3) δ 167.2 (C-6), 151.7 (C-2), 151.6 (C-4), 124.5 (C-8), 117.1 (C-5), 100.4 (C-7), 45.4 (CH(CH2)4), 32.8, 32.8, 26.5, 26.5 (4×CH2); HRMS (ESI-TOF) m/z: calcd for C11H13N3 ([M+H]+), 188.1182, found 188.1182.

Example 11. Synthesis of 6-Propyl-7-deazapurine (5k). Following the general procedure, compound 5k was obtained starting from 6-chloro-7-deazapurine (4) (200 mg, 1.3 mmol), FeCl3 (21 mg, 1.3 mmol), propylmagnesium chloride 2 M in THF (370.24 mg, 3.6 mmol, added as portions and TLC monitor) in dry THF (5.0 mL) and NMP (0.5 mL) after column chromatography on silica gel (Heptane/EtOAc=5:1, to DCM:MeOH=30:1, v/v) as white solid (170 mg, 82% yield). 1H NMR (300 MHz, CD3OD) δ 8.62 (s, 1H, H-2), 7.42 (d, J8,7=3.6 Hz, 1H, H-8), 6.69 (d, J7,8=3.6 Hz, 1H, H-7), 3.03 (t, J=7.2 Hz, 2H, CH2CH2), 1.91-1.83 (m, 2H, CH2CH3), 1.00 (t, J=7.7 Hz, 3H, CH2CH3); 13C{1H} NMR (75 MHz, CD3OD) δ 162.2 (C-6), 150.6 (C-2), 149.6 (C-4), 125.5 (C-8), 117.0 (C-5), 98.9 (C-7), 36.2 (CH2CH2), 21.7 (CH2CH3), 12.5 (CH3); HRMS (ESI-TOF) m/z: calcd for C9H11N3 ([M+H]+), 162.1025, found 162.1029.

Example 12. Synthesis of 2′,3′,5′-Tri-O-benzoyl-6-chloro-9-β-D-ribofuranosyl-7-deazapurine (9). To a solution of compound 824 (9 g, 12.5 mmol) in dry THF (50 mL) was dropwise added iPrMgCl.LiCl (1.3 M in THF, 1.89 g, 13 mmol) at −10° C. and the solution was stirred at this temperature yet for 30 min. Then the reaction mixture was poured on the mixture of ice and saturated aq. NH4Cl (100 mL) and was extracted with EtOAc (200 mL, then 3×20 mL). Combined organic phases were dried over Na2SO4 and evaporated to dryness in vacuo. Purification by silica gel chromatography to get 9 g compound 9 (5 g, 71%) as a foam. 1H NMR (300 MHz, CDCl3) δ 8.60 (s, 1H, H-2), 8.12-7.19 (m, 6H, Ph-H), 7.60-7.32 (m, 10H, H-8, Ph-H), 6.68 (d, J=5.6 Hz, 1H, H-1′), 6.62 (d, J7,8=3.7 Hz, 1H, H-7), 6.25 (dd, J2′,1′=5.6 Hz, J2′,3′=5.0 Hz, 1H, H-2′), 6.15 (dd, J3′,2′=5.0 Hz, J3′,4′=4.3 Hz, 1H, H-3′), 4.89 (dd, J5′,4′=3.2 Hz, Jgem=11.9 Hz, 1H, H-5′), 4.81 (ddd, J4′,3′=4.3 Hz, J4′,5′=3.2 Hz, J4′,5″=3.7 Hz, 1H, H-4′), 4.68 (dd, J5″,4′=3.7 Hz, Jgem=11.9 Hz, 1H, H-5″); 13C{1H} NMR (75 MHz, CDCl3) δ 166.4 (COOPh), 165.7 (COOPh), 165.4 (COOPh), 152.8 (C-6), 151.8 (C-4), 151.4 (C-2), 134.0 (C-Ph), 133.7 (C-Ph), 130.1 (C-Ph), 130.0 (C-Ph), 129.7 (C-Ph), 129.1 (C-Ph), 128.9 (C-Ph), 128.8 (C-Ph), 127.0 (C-8), 118.9 (C-5), 101.7 (C-7), 87.2 (C-1′), 80.7 (C-4′), 74.3 (C-2′), 71.8 (C-3′), 64.0 (C-5′);

General Procedure for the Fe(acac)3/CuI Catalyzed Cross-Coupling of 2′,3′,5′-tri-O-benzoyl-6-chloro-9-β-D-ribofuranosyl-7-desazapurine (9) with Grignard Reagents. An oven-dried flask was charged with 2′,3′,5′-tri-O-benzoyl-6-chloro-9-β-D-ribofuranosyl-7-desazapurine (9) (0.2 mmol, 1 equiv) in THF 5 mL, NMP 0.5 mL, Fe(acac)3 (0.02 mmol, 0.1 equiv), CuI (0.04 mol, 0.2 equiv). A solution of RMgX (0.5-1.8 mmol, 2.5-9.0 equiv) in THF was added in ice bath. The reaction mixture was stirred for 30 mins in ice bath. Monitoring with TLC till starting material disappeared, the reaction was quenched by the addition of sat. aq solution of NH4Cl and extracted with EtOAc (3×10 mL). The organic solution was dried over Na2SO4 and concentrated under reduced pressure. The crude product was purified by silica gel chromatography to afford the desired product.

Example 13. Synthesis of 2′,3′,5′-Tri-O-benzoyl-6-(4-methylphenyl)-9-β-D-ribofuranosyl-7-deazapurine (10a). Following the general procedure, compound 10a was obtained starting from 2′,3′,5′-tri-O-benzoyl-6-chloro-9-β-D-ribofuranosyl-7-desazapurine (9) (120 mg, 0.2 mmol), Fe(acac)3 (7 mg, 0.02 mmol), 4-methylphenylmagnesium bromide 1 M in THF (128.25 mg, 0.85 mmol, added as portions and TLC monitor) in dry THF (5.0 mL) and NMP (0.5 mL) after column chromatography on silica gel (Heptane/EtOAc=5:1, to 2:1, v/v) as white foam (75 mg, 55% yield). 1H NMR (300 MHz, CDCl3) δ 8.95 (s, 1H, H-2), 8.13 (d, J=7.4 Hz, 2H, Ph-H), 8.02-7.93 (m, 6H, Ph-H), 7.58-7.32 (m, 12H, H-8, Ph-H), 6.82 (d, J7,8=3.5 Hz, 1H, H-7), 6.81 (d, J=5.6 Hz, 1H, H-1′), 6.30 (dd, J2′,1′=5.6 Hz, J2′,3′=5.0 Hz, 1H, H-2′), 6.19 (dd, J3′,2′=5.0 Hz, J3′,4′=4.2 Hz, 1H, H-3′), 4.89 (dd, J5′,4′=3.0 Hz, Jgem=11.9 Hz, 1H, H-5′), 4.81 (ddd, J4′,3′=4.3 Hz, J4′,5′=3.2 Hz, J4′,5″=3.7 Hz, 1H, H-4′), 4.70 (dd, J5″,4′=3.7 Hz, Jgem=11.9 Hz, 1H, H-5″), 2.43 (s, 3H, CH3); 13C{1H} NMR (75 MHz, CDCl3) δ 166.4 (COOPh), 165.7 (COOPh), 165.4 (COOPh), 158.4 (C-6), 152.6 (C-4), 152.2 (C-2), 151.2 (C-Ph), 140.7 (C-Ph), 135.3 (C-Ph), 133.9 (C-Ph), 133.7 (C-Ph), 130.1 (C-Ph), 130.0 (C-Ph), 129.8 (C-Ph), 129.1 (C-Ph), 128.9 (C-Ph), 128.8 (C-Ph), 128.7 (C-Ph), 126.1 (C-8), 116.8 (C-5), 103.0 (C-7), 86.6 (C-1′), 80.5 (C-4′), 74.2 (C-2′), 71.9 (C-3′), 64.2 (C-5′), 21.7 (CH3); HRMS (ESI-TOF) m/z: calcd for C39H31N3O7 ([M+H]+), 654.2234, found 654.2250.

Example 14. Synthesis of 2′,3′,5′-Tri-O-benzoyl-6-methyl-9-β-D-ribofuranosyl-7-deazapurine (10b). Following the general procedure, compound 10b was obtained starting from 2′,3′,5′-tri-O-benzoyl-6-chloro-9-β-D-ribofuranosyl-7-desazapurine (9) (120 mg, 0.2 mmol), Fe(acac)3 (7 mg, 0.02 mmol), CuI (8 mg, 0.04 mol), methylmagnesium bromide 3 M in THF (75.12 mg, 0.63 mmol, added as portions and TLC monitor) in dry THF (5.0 mL) and NMP (0.5 mL) after column chromatography on silica gel (Heptane/EtOAc=5:1, to 2:1, v/v) as white foam (70 mg, 61% yield). 1H NMR (300 MHz, CDCl3) δ 8.75 (s, 1H, H-2), 8.14 (d, J=7.9 Hz, 2H, Ph-H), 8.01 (d, J=7.9 Hz, 2H, Ph-H), 7.93 (d, J=7.9 Hz, 2H, Ph-H), 7.58-7.31 (m, 10H, H-8, Ph-H), 6.75 (d, J=5.5 Hz, 1H, H-1′), 6.59 (d, J7,8=3.7 Hz, 1H, H-7), 6.27 (dd, J2′,1′=5.5 Hz, J2′,3′=5.0 Hz, 1H, H-2′), 6.18 (dd, J3′,2′=5.0 Hz, J3′,4′=4.2 Hz, 1H, H-3′), 4.88 (dd, J5′,4′=3.0 Hz, Jgem=11.9 Hz, 1H, H-5′), 4.80 (ddd, J4′,3′=4.2 Hz, J4′,5′=3.0 Hz, J4′,5″=3.8 Hz, 1H, H-4′), 4.69 (dd, J5″,4′=3.8 Hz, Jgem=11.9 Hz, 1H, H-5″), 2.69 (s, 3H, CH3); 13C{1H} NMR (75 MHz, CDCl3) δ 166.4 (COOPh), 165.7 (COOPh), 165.4 (COOPh), 160.1 (C-6), 151.9 (C-2), 151.1 (C-4), 133.9 (C-Ph), 133.7 (C-Ph), 130.1 (C-Ph), 130.0 (C-Ph), 129.7 (C-Ph), 129.1 (C-Ph), 128.9 (C-Ph), 128.8 (C-Ph), 128.7 (C-Ph), 125.3 (C-8), 119.0 (C-5), 101.7 (C-7), 86.5 (C-1′), 80.4 (C-4′), 74.2 (C-2′), 71.9 (C-3′), 64.2 (C-5′), 21.8 (CH3); HRMS (ESI-TOF) m/z: calcd for C33H27N3O7 ([M+H]+), 578.1921, found 578.1931.

Example 15. Synthesis of 2′,3′,5′-Tri-O-benzoyl-6-isopropyl-9-β-D-ribofuranosyl-7-deazapurine (10c). Following the general procedure, compound 10c was obtained starting from 2′,3′,5′-tri-O-benzoyl-6-chloro-9-β-D-ribofuranosyl-7-desazapurine (9) (140 mg, 0.234 mmol), Fe(acac)3 (8 mg, 0.0234 mmol), CuI (9 mg, 0.046 mol), isopropylmagnesium bromide 3 M in THF (88.38 mg, 0.6 mmol, added as portions and TLC monitor) in dry THF (5.0 mL) and NMP (0.5 mL) after column chromatography on silica gel (Heptane/EtOAc=5:1, to 2:1, v/v) as white foam (100 mg, 71% yield). 1H NMR (300 MHz, CDCl3) δ 8.83 (s, 1H, H-2), 8.14 (d, J=7.5 Hz, 2H, Ph-H), 7.99 (d, J=7.5 Hz, 2H, Ph-H), 7.94 (d, J=7.5 Hz, 2H, Ph-H), 7.65-7.30 (m, 10H, H-8, Ph-H), 6.77 (d, J=5.7 Hz, 1H, H-1′), 6.61 (d, J7,8=3.7 Hz, 1H, H-7), 6.26 (dd, J2′,1′=5.7 Hz, J2′,3′=5.0 Hz, 1H, H-2′), 6.16 (dd, J3′,2′=5.2 Hz, J3′,4′=4.3 Hz, 1H, H-3′), 4.89 (dd, J5′,4′=3.1 Hz, Jgem=11.9 Hz, 1H, H-5′), 4.79 (ddd, J4′,3′=4.3 Hz, J4′,5′=3.1 Hz, J4′,5″=3.6 Hz, 1H, H-4′), 4.69 (dd, J5″,4′=3.6 Hz, Jgem=11.9 Hz, 1H, H-5″), 3.44-3.36 (m, 1H, CH(CH3)2), 1.40 (s, 3H, CH3), 1.37 (s, 3H, CH3); 13C{1H} NMR (75 MHz, CDCl3) δ 168.6 (C-6), 166.4 (COOPh), 165.7 (COOPh), 165.4 (COOPh), 152.6 (C-2), 151.6 (C-4), 133.9 (C-Ph), 133.6 (C-Ph), 130.1 (C-Ph), 130.0 (C-Ph), 129.8 (C-Ph), 129.1 (C-Ph), 128.8 (C-Ph), 128.7 (C-Ph), 125.0 (C-8), 117.4 (C-5), 101.5 (C-7), 86.5 (C-1′), 80.4 (C-4′), 74.1 (C-2′), 71.9 (C-3′), 64.2 (C-5′), 34.1 (CH(CH3)2), 21.7 (CH(CH3)2), 21.6 (CH(CH3)2); HRMS (ESI-TOF) m/z: calcd for C35H31N3O7 ([M+H]+), 606.2234, found 606.2233.

Example 16. Synthesis of 2′,3′,5′-Tri-O-benzoyl-6-ethyl-9-β-D-ribofuranosyl-7-deazapurine (10d). Following the general procedure, compound 10d was obtained starting from 2′,3′,5′-tri-O-benzoyl-6-chloro-9-β-D-ribofuranosyl-7-desazapurine (9) (140 mg, 0.234 mmol), Fe(acac)3 (8 mg, 0.0234 mmol), CuI (9 mg, 0.046 mol), ethylmagnesium bromide 3 M in THF (67.97 mg, 0.51 mmol, added as portions and TLC monitor) in dry THF (5.0 mL) and NMP (0.5 mL) after column chromatography on silica gel (Heptane/EtOAc=5:1, to 2:1, v/v) as white foam (100 mg, 72% yield). 1H NMR (300 MHz, CDCl3) δ 8.79 (s, 1H, H-2), 8.14 (d, J=8.1 Hz, 2H, Ph-H), 8.00 (d, J=8.1 Hz, 2H, Ph-H), 7.94 (d, J=8.1 Hz, 2H, Ph-H), 7.60-7.32 (m, 10H, H-8, Ph-H), 6.75 (d, J=5.8 Hz, 1H, H-1′), 6.59 (d, J7,8=3.6 Hz, 1H, H-7), 6.26 (dd, J2′,1′=5.8 Hz, J2′,3′=5.3 Hz, 1H, H-2′), 6.17 (dd, J3′,2′=5.3 Hz, J3′,4′=4.2 Hz, 1H, H-3′), 4.87 (dd, J5′,4′=3.2 Hz, Jgem=11.9 Hz, 1H, H-5′), 4.79 (ddd, J4′,3′=4.2 Hz, J4′,5′=3.2 Hz, J4′,5″=3.9 Hz, 1H, H-4′), 4.69 (dd, J5″,4′=3.9 Hz, Jgem=11.9 Hz, 1H, H-5″), 2.96 (q, J=7.6 Hz, 2H, CH2), 1.38 (t, J=7.6 Hz, 3H, CH3); 13C{1H} NMR (75 MHz, CDCl3) δ 166.4 (COOPh), 165.7 (COOPh), 165.4 (COOPh), 164.9 (C-6), 152.1 (C-2), 151.4 (C-4), 133.9 (C-Ph), 133.6 (C-Ph), 130.1 (C-Ph), 130.0 (C-Ph), 129.8 (C-Ph), 129.1 (C-Ph), 128.8 (C-Ph), 128.7 (C-Ph), 125.2 (C-8), 118.2 (C-5), 101.6 (C-7), 86.6 (C-1′), 80.4 (C-4′), 74.1 (C-2′), 71.9 (C-3′), 64.2 (C-5′), 28.8 (CH2), 13.0 (CH3); HRMS (ESI-TOF) m/z: calcd for C34H29N3O7 ([M+H]+), 592.2078, found 592.2092.

Example 17. Synthesis of 2′,3′,5′-Tri-O-benzoyl-6-propyl-9-β-D-ribofuranosyl-7-deazapurine (10e). Following the general procedure, compound 10e was obtained starting from 2′,3′,5′-tri-O-benzoyl-6-chloro-9-β-D-ribofuranosyl-7-desazapurine (9) (120 mg, 0.2 mmol), Fe(acac)3 (7 mg, 0.02 mmol), CuI (8 mg, 0.043 mol), propylmagnesium chloride 2 M in THF (53.48 mg, 0.54 mmol, added as portions and TLC monitor) in dry THF (5.0 mL) and NMP (0.5 mL) after column chromatography on silica gel (Heptane/EtOAc=5:1, to 2:1, v/v) as white foam (64 mg, 53% yield). 1H NMR (300 MHz, CDCl3) δ 8.79 (s, 1H, H-2), 8.13 (d, J=7.4 Hz, 2H, Ph-H), 8.00 (d, J=8.3 Hz, 2H, Ph-H), 7.94 (d, J=8.3 Hz, 2H, Ph-H), 7.59-7.31 (m, 10H, H-8, Ph-H), 6.76 (d, J=5.7 Hz, 1H, H-1′), 6.58 (d, J7,8=3.8 Hz, 1H, H-7), 6.28 (dd, J2′,1′=5.7 Hz, J2′,3′=5.1 Hz, 1H, H-2′), 6.19 (dd, J3′,2′=5.1 Hz, J3′,4′=4.2 Hz, 1H, H-3′), 4.88 (dd, J5′,4′=3.1 Hz, Jgem=11.9 Hz, 1H, H-5′), 4.80 (ddd, J4′,3′=4.2 Hz, J4′,5′=3.0 Hz, J4′,5″=3.9 Hz, 1H, H-4′), 4.69 (dd, J5″,4′=3.9 Hz, Jgem=11.9 Hz, 1H, H-5″), 2.96 (t, J=7.0 Hz, 2H, CH2CH2), 1.89-1.81 (m, 2H, CH2CH3), 0.98 (t, J=7.6 Hz, 3H, CH2CH3); 13C{1H} NMR (75 MHz, CDCl3) δ 166.4 (COOPh), 165.7 (COOPh), 165.4 (COOPh), 163.9 (C-6), 152.1 (C-2), 151.4 (C-4), 133.9 (C-Ph), 133.6 (C-Ph), 130.1 (C-Ph), 130.0 (C-Ph), 129.8 (C-Ph), 129.1 (C-Ph), 128.8 (C-Ph), 128.7 (C-Ph), 125.3 (C-8), 118.8 (C-5), 101.6 (C-7), 86.6 (C-1′), 80.4 (C-4′), 74.2 (C-2′), 71.9 (C-3′), 64.2 (C-5′), 37.6 (CH2CH2), 22.3 (CH2CH2), 14.3 (CH2CH3); HRMS (ESI-TOF) m/z: calcd for C35H31N3O7 ([M+H]+), 606.2234, found 606.2230.

Example 18. Synthesis of 2′,3′,5′-Tri-O-benzoyl-6-pentyl-9-β-D-ribofuranosyl-7-deazapurine (10f). Following the general procedure, compound 10f was obtained starting from 2′,3′,5′-tri-O-benzoyl-6-chloro-9-β-D-ribofuranosyl-7-desazapurine (9) (110 mg, 0.183 mmol), Fe(acac)3 (6 mg, 0.0183 mmol), CuI (7 mg, 0.036 mol), pentylmagnesium bromide 2 M in THF (157.82 mg, 0.9 mmol, added as portions and TLC monitor) in dry THF (5.0 mL) and NMP (0.5 mL) after column chromatography on silica gel (Heptane/EtOAc=5:1, to 2:1, v/v) as white foam (80 mg, 67% yield). 1H NMR (300 MHz, CDCl3) δ 8.79 (s, 1H, H-2), 8.13 (d, J=7.6 Hz, 2H, Ph-H), 8.00 (d, J=7.6 Hz, 2H, Ph-H), 7.94 (d, J=7.2 Hz, 2H, Ph-H), 7.59-7.32 (m, 10H, H-8, Ph-H), 6.75 (d, J=5.8 Hz, 1H, H-1′), 6.82 (d, J7,8=3.8 Hz, 1H, H-7), 6.27 (dd, J2′,1′=5.8 Hz, J2′,3′=5.3 Hz, 1H, H-2′), 6.19 (dd, J3′,2′=5.3 Hz, J3′,4′=4.3 Hz, 1H, H-3′), 4.89 (dd, J5′,4′=3.1 Hz, Jgem=11.9 Hz, 1H, H-5′), 4.81 (ddd, J4′,3′=4.3 Hz, J4′,5′=3.1 Hz, J4′,5″=3.8 Hz, 1H, H-4′), 4.70 (dd, J5″,4′=3.8 Hz, Jgem=11.9 Hz, 1H, H-5″), 2.97 (t, J=7.2 Hz, 2H, CH2(CH2)3), 1.84-1.79 (m, 2H, CH2CH3), 1.38-1.33 (m, 4H, CH2(CH2)2), 0.90-0.85 (t, J=7.0 Hz, 3H, (CH2)4CH3); 13C{1H} NMR (75 MHz, CDCl3) δ 166.4 (COOPh), 165.7 (COOPh), 165.4 (COOPh), 164.1 (C-6), 152.1 (C-2), 151.4 (C-4), 133.9 (C-Ph), 133.7 (C-Ph), 130.1 (C-Ph), 130.0 (C-Ph), 129.7 (C-Ph), 129.1 (C-Ph), 128.9 (C-Ph), 128.8 (C-Ph), 128.7 (C-Ph), 125.3 (C-8), 118.7 (C-5), 101.6 (C-7), 86.5 (C-1′), 80.4 (C-4′), 74.1 (C-2′), 71.8 (C-3′), 64.2 (C-5′), 35.7, 32.0, 28.8, 22.7, 14.2 (aliphatic chain); HRMS (ESI-TOF) m/z: calcd for C37H35N3O7 ([M+H]+), 634.2547, found 634.2552.

Example 19. Synthesis of 2′,3′,5′-Tri-O-benzoyl-6-hexyl-9-β-D-ribofuranosyl-7-deazapurine (10g). Following the general procedure, compound 10g was obtained starting from 2′,3′,5′-tri-O-benzoyl-6-chloro-9-β-D-ribofuranosyl-7-desazapurine (9) (120 mg, 0.2 mmol), Fe(acac)3 (7 mg, 0.02 mmol), CuI (8 mg, 0.043 mol), hexylmagnesium bromide 2 M in THF (170.44 mg, 0.9 mmol, added as portions and TLC monitor) in dry THF (5.0 mL) and NMP (0.5 mL) after column chromatography on silica gel (Heptane/EtOAc=5:1, to 2:1, v/v) as white foam (86 mg, 67% yield). 1H NMR (300 MHz, CDCl3) δ 8.79 (s, 1H, H-2), 8.13 (d, J=7.6 Hz, 2H, Ph-H), 8.00 (d, J=7.6 Hz, 2H, Ph-H), 7.94 (d, J=7.6 Hz, 2H, Ph-H), 7.59-7.32 (m, 10H, H-8, Ph-H), 6.75 (d, J=5.7 Hz, 1H, H-1′), 6.58 (d, J7,8=3.8 Hz, 1H, H-7), 6.27 (dd, J2′,1′=5.7 Hz, J2′,3′=5.2 Hz, 1H, H-2′), 6.19 (dd, J3′,2′=5.2 Hz, J3′,4′=4.3 Hz, 1H, H-3′), 4.89 (dd, J5′,4′=3.2 Hz, Jgem=11.9 Hz, 1H, H-5′), 4.81 (ddd, J4′,3′=4.3 Hz, J4′,5′=3.1 Hz, J4′,5″=3.8 Hz, 1H, H-4′), 4.70 (dd, J5″,4′=3.8 Hz, Jgem=11.9 Hz, 1H, H-5″), 2.97 (t, J=7.2 Hz, 2H, CH2(CH2)3), 1.85-1.75 (m, 2H, CH2CH3), 1.40-1.27 (m, 6H, (CH2)3CH3), 0.88-0.83 (t, J=7.0 Hz, 3H, (CH2)4CH3); 13C{1H} NMR (75 MHz, CDCl3) δ 166.4 (COOPh), 165.7 (COOPh), 165.4 (COOPh), 164.1 (C-6), 152.0 (C-2), 151.4 (C-4), 133.9 (C-Ph), 133.6 (C-Ph), 130.1 (C-Ph), 130.0 (C-Ph), 129.8 (C-Ph), 129.1 (C-Ph), 128.8 (C-Ph), 128.7 (C-Ph), 125.3 (C-8), 118.6 (C-5), 101.6 (C-7), 86.6 (C-1′), 80.4 (C-4′), 74.2 (C-2′), 71.9 (C-3′), 64.2 (C-5′), 35.7, 31.8, 29.5, 29.0, 22.7, 14.3 (aliphatic chain); HRMS (ESI-TOF) m/z: calcd for C38H37N3O7 ([M+H]+), 648.2704, found 648.2720.

Example 20. Synthesis of 2′,3′,5′-Tri-O-benzoyl-6-(but-3-en-1-yl)-9-β-D-ribofuranosyl-7-desazapurine (10h). Following the general procedure, compound 10h was obtained starting from 2′,3′,5′-tri-O-benzoyl-6-chloro-9-β-D-ribofuranosyl-7-desazapurine (9) (120 mg, 0.2 mmol), Fe(acac)3 (7 mg, 0.02 mmol), CuI (8 mg, 0.043 mol), 3-butenylmagnesium bromide 0.5 M in THF (286.76 mg, 1.8 mmol, added as portions and TLC monitor) in dry THF (5.0 mL) and NMP (0.5 mL) after column chromatography on silica gel (Heptane/EtOAc=5:1, to 2:1, v/v) as white foam (50 mg, 41% yield). 1H NMR (300 MHz, CDCl3) δ 8.79 (s, 1H, H-2), 8.13 (d, J=7.7 Hz, 2H, Ph-H), 8.00 (d, J=7.7 Hz, 2H, Ph-H), 7.94 (d, J=7.7 Hz, 2H, Ph-H), 7.60-7.32 (m, 10H, H-8, Ph-H), 6.75 (d, J=5.7 Hz, 1H, H-1′), 6.58 (d, J7,8=3.8 Hz, 1H, H-7), 6.25 (dd, J2′,1′=5.7 Hz, J2′,3′=5.2 Hz, 1H, H-2′), 6.19 (dd, J3′,2′=5.2 Hz, J3′,4′=4.5 Hz, 1H, H-3′), 5.94-5.81 (m, 1H, CH═CH2), 5.07 (d, J=17.1 Hz, 1H, CH═CH′), 4.97 (d, J=17.1 Hz, 1H, CH═CH″), 4.88 (dd, J5′,4′=3.0 Hz, Jgem=11.9 Hz, 1H, H-5′), 4.81 (ddd, J4′,3′=4.5 Hz, J4′,5′=3.0 Hz, J4′,5″=3.9 Hz, 1H, H-4′), 4.70 (dd, J5″,4′=3.9 Hz, Jgem=11.9 Hz, 1H, H-5″), 3.07 (t, J=7.1 Hz, 2H, CH2), 2.65-2.55 (m, 2H, CH2); 13C{1H} NMR (75 MHz, CDCl3) δ 166.4 (COOPh), 165.7 (COOPh), 165.4 (COOPh), 163.0 (C-6), 152.1 (C-2), 151.4 (C-4), 137.6 (CH═CH2), 133.9 (C-Ph), 133.6 (C-Ph), 130.1 (C-Ph), 130.0 (C-Ph), 129.8 (C-Ph), 129.1 (C-Ph), 128.8 (C-Ph), 128.7 (C-Ph), 125.4 (C-8), 118.7 (C-5), 115.6 (CH═CH2), 101.6 (C-7), 86.6 (C-1′), 80.4 (C-4′), 74.2 (C-2′), 71.8 (C-3′), 64.2 (C-5′), 35.0 (CH2), 32.7 (CH2); HRMS (ESI-TOF) m/z: calcd for C36H31N3O7 ([M+H]+), 618.2234, found 618.2228.

Example 21. Synthesis of 2′,3′,5′-Tri-O-benzoyl-6-isobutyl-9-β-D-ribofuranosyl-7-deazapurine (10i). Following the general procedure, compound 10i was obtained starting from 2′,3′,5′-tri-O-benzoyl-6-chloro-9-β-D-ribofuranosyl-7-desazapurine (9) (120 mg, 0.2 mmol), Fe(acac)3 (7 mg, 0.02 mmol), CuI (8 mg, 0.043 mol), isobutylmagnesium bromide 2 M in THF (241.99 mg, 1.5 mmol, added as portions and TLC monitor) in dry THF (5.0 mL) and NMP (0.5 mL) after column chromatography on silica gel (Heptane/EtOAc=5:1, to 2:1, v/v) as white foam (84 mg, 68% yield). 1H NMR (300 MHz, CDCl3) δ 8.79 (s, 1H, H-2), 8.13 (d, J=7.6 Hz, 2H, Ph-H), 8.00 (d, J=7.6 Hz, 2H, Ph-H), 7.94 (d, J=7.6 Hz, 2H, Ph-H), 7.59-7.32 (m, 10H, H-8, Ph-H), 6.75 (d, J=5.6 Hz, 1H, H-1′), 6.57 (d, J7,8=3.8 Hz, 1H, H-7), 6.27 (dd, J2′,1′=5.6 Hz, J2′,3′=5.2 Hz, 1H, H-2′), 6.17 (dd, J3′,2′=5.2 Hz, J3′,4′=4.3 Hz, 1H, H-3′), 4.89 (dd, J5′,4′=3.1 Hz, Jgem=11.9 Hz, 1H, H-5′), 4.79 (ddd, J4′,3′=4.3 Hz, J4′,5′=3.1 Hz, J4′,5″=3.8 Hz, 1H, H-4′), 4.70 (dd, J5″,4′=3.8 Hz, Jgem=11.9 Hz, 1H, H-5″), 2.85 (d, J=7.2 Hz, 2H, CH2CH), 2.31-2.22 (m, 1H, CH(CH3)2), 0.97-0.94 (m, 6H, CH(CH3)2); 13C{1H} NMR (75 MHz, CDCl3) δ 166.4 (COOPh), 165.7 (COOPh), 165.4 (COOPh), 163.3 (C-6), 152.0 (C-2), 151.4 (C-4), 133.9 (C-Ph), 133.6 (C-Ph), 130.1 (C-Ph), 130.0 (C-Ph), 129.8 (C-Ph), 129.1 (C-Ph), 128.8 (C-Ph), 128.7 (C-Ph), 125.3 (C-8), 119.3 (C-5), 101.7 (C-7), 86.6 (C-1′), 80.4 (C-4′), 74.2 (C-2′), 71.9 (C-3′), 64.2 (C-5′), 44.7 (CH2CH), 29.1 CH(CH3)2, 23.0(CH3); HRMS (ESI-TOF) m/z: calcd for C36H33N3O7 ([M+H]+), 620.2391, found 620.2413.

Example 22. Synthesis of 2′,3′,5′-Tri-O-benzoyl-6-cyclopropyl-9-β-D-ribofuranosyl-7-deazapurine (10j). Following the general procedure, compound 10j was obtained starting from 2′,3′,5′-tri-O-benzoyl-6-chloro-9-β-D-ribofuranosyl-7-desazapurine (9) (130 mg, 0.217 mmol), Fe(acac)3 (8 mg, 0.0217 mmol), CuI (9 mg, 0.043 mol), cyclopropylmagnesium bromide 0.7 M in THF (209.85 mg, 1.1 mmol, added as portions and TLC monitor) in dry THF (5.0 mL) and NMP (0.5 mL) after column chromatography on silica gel (Heptane/EtOAc=5:1, to 2:1, v/v) as white foam (95 mg, 73% yield). 1H NMR (300 MHz, CDCl3) δ 8.68 (s, 1H, H-2), 8.13 (d, J=7.4 Hz, 2H, Ph-H), 7.99 (d, J=7.4 Hz, 2H, Ph-H), 7.93 (d, J=7.4 Hz, 2H, Ph-H), 7.65-7.30 (m, 10H, H-8, Ph-H), 6.75 (d, J=5.9 Hz, 1H, H-1′), 6.65 (d, J7,8=3.8 Hz, 1H, H-7), 6.25 (dd, J2′,1′=5.9 Hz, J2′,3′=5.3 Hz, 1H, H-2′), 6.15 (dd, J3′,2′=5.3 Hz, J3′,4′=4.2 Hz, 1H, H-3′), 4.86 (dd, J5′,4′=3.0 Hz, Jgem=11.9 Hz, 1H, H-5′), 4.79 (ddd, J4′,3′=4.2 Hz, J4′,5′=3.0 Hz, J4′,5″=3.8 Hz, 1H, H-4′), 4.69 (dd, J5″,4′=3.8 Hz, Jgem=11.9 Hz, 1H, H-5″), 2.34-2.26 (m, 1H, CH), 1.34-1.11 1.32-1.41 (m, 4H, CH(CH2)2); 13C{1H} NMR (75 MHz, CDCl3) δ 166.4 (COOPh), 165.7 (COOPh), 165.4 (COOPh), 165.1 (C-6), 152.2 (C-2), 150.8 (C-4), 133.8 (C-Ph), 133.6 (C-Ph), 130.1 (C-Ph), 130.0 (C-Ph), 129.8 (C-Ph), 129.1 (C-Ph), 128.8 (C-Ph), 128.7 (C-Ph), 124.8 (C-8), 118.3 (C-5), 101.4 (C-7), 86.4 (C-1′), 80.4 (C-4′), 74.2 (C-2′), 71.9 (C-3′), 64.2 (C-5′), 14.8 (CH(CH2)2), 11.1 (2×CH2); HRMS (ESI-TOF) m/z: calcd for C35H29N3O7 ([M+H]+), 604.2078, found 604.2097.

Example 23. Synthesis of 2′,3′,5′-Tri-O-benzoyl-6-cyclopentyl-9-β-D-ribofuranosyl-7-deazapurine (10k). Following the general procedure, compound 10k was obtained starting from 2′,3′,5′-tri-O-benzoyl-6-chloro-9-β-D-ribofuranosyl-7-desazapurine (9) (120 mg, 0.2 mmol), Fe(acac)3 (8 mg, 0.02 mmol), CuI (9 mg, 0.04 mol), cyclopentylmagnesium bromide 1 M in THF (112.67 mg, 0.65 mmol, added as portions and TLC monitor) in dry THF (5.0 mL) and NMP (0.5 mL) after column chromatography on silica gel (Heptane/EtOAc=5:1, to 2:1, v/v) as white foam (80 mg, 62% yield). 1H NMR (300 MHz, CDCl3) δ 8.82 (s, 1H, H-2), 8.14 (d, J=7.8 Hz, 2H, Ph-H), 8.00 (d, J=7.8 Hz, 2H, Ph-H), 7.94 (d, J=7.8 Hz, 2H, Ph-H), 7.59-7.32 (m, 10H, H-8, Ph-H), 6.78 (d, J=5.8 Hz, 1H, H-1′), 6.60 (d, J7,8=3.5 Hz, 1H, H-7), 6.28 (dd, J2′,1′=5.8 Hz, J2′,3′=5.0 Hz, 1H, H-2′), 6.17 (dd, J3′,2′=5.0 Hz, J3′,4′=4.2 Hz, 1H, H-3′), 4.88 (dd, J5′,4′=3.0 Hz, Jgem=11.9 Hz, 1H, H-5′), 4.79 (ddd, J4′,3′=4.2 Hz, J4′,5′=3.0 Hz, J4′,5″=3.5 Hz, 1H, H-4′), 4.69 (dd, J5″,4′=3.5 Hz, Jgem=11.9 Hz, 1H, H-5″), 3.53-3.47 (m, 1H, CH), 2.08-1.73 (m, 8H, (CH2)4); 13C{1H} NMR (75 MHz, CDCl3) δ 167.4 (C-6), 166.4 (COOPh), 165.7 (COOPh), 165.4 (COOPh), 152.1 (C-2), 151.4 (C-4), 133.9 (C-Ph), 133.6 (C-Ph), 130.1 (C-Ph), 130.0 (C-Ph), 129.8 (C-Ph), 129.1 (C-Ph), 128.8 (C-Ph), 128.7 (C-Ph), 125.0 (C-8), 118.2 (C-5), 101.7 (C-7), 86.5 (C-1′), 80.4 (C-4′), 74.1 (C-2′), 71.9 (C-3′), 64.2 (C-5′), 45.1 (CH(CH2)4), 32.9, 32.8, 26.5, 26.5 (4×CH2); HRMS (ESI-TOF) m/z: calcd for C37H33N3O7 ([M+H]+), 632.2391, found 632.2407.

Example 24. Synthesis of 2′,3′,5′-Tri-O-benzoyl-6-cyclohexyl-9-β-D-ribofuranosyl-7-deazapurine (10l). Following the general procedure, compound 10l was obtained starting from 2′,3′,5′-tri-O-benzoyl-6-chloro-9-β-D-ribofuranosyl-7-desazapurine (9) (120 mg, 0.2 mmol), Fe(acac)3 (7 mg, 0.02 mmol), CuI (8 mg, 0.043 mol), cyclohexylmagnesium bromide 1 M in THF (121.79 mg, 0.65 mmol, added as portions and TLC monitor) in dry THF (5.0 mL) and NMP (0.5 mL) after column chromatography on silica gel (Heptane/EtOAc=5:1, to 2:1, v/v) as white foam (80 mg, 62% yield). 1H NMR (300 MHz, CDCl3) δ 8.81 (s, 1H, H-2), 8.14 (d, J=7.3 Hz, 2H, Ph-H), 8.00 (d, J=7.3 Hz, 2H, Ph-H), 7.94 (d, J=7.3 Hz, 2H, Ph-H), 7.60-7.30 (m, 10H, H-8, Ph-H), 6.76 (d, J=5.9 Hz, 1H, H-1′), 6.62 (d, J7,8=3.7 Hz, 1H, H-7), 6.25 (dd, J2′,1′=5.9 Hz, J2′,3′=5.0 Hz, 1H, H-2′), 6.16 (dd, J3′,2′=5.0 Hz, J3′,4′=4.3 Hz, 1H, H-3′), 4.86 (dd, J5′,4′=3.1 Hz, Jgem=11.9 Hz, 1H, H-5′), 4.78 (ddd, J4′,3′=4.3 Hz, J4′,5′=3.1 Hz, J4′,5″=3.8 Hz, 1H, H-4′), 4.69 (dd, J5″,4′=3.5 Hz, Jgem=11.9 Hz, 1H, H-5″), 3.07-2.99 (m, 1H, CH(CH2)5), 1.90-1.35 (m, 10H, CH(CH2)5); 13C{1H} NMR (75 MHz, CDCl3) δ 167.7 (C-6), 166.4 (COOPh), 165.7 (COOPh), 165.4 (COOPh), 152.1 (C-2), 151.6 (C-4), 133.9 (C-Ph), 133.6 (C-Ph), 130.1 (C-Ph), 130.0 (C-Ph), 129.8 (C-Ph), 129.1 (C-Ph), 128.8 (C-Ph), 128.7 (C-Ph), 124.9 (C-8), 117.7 (C-5), 101.6 (C-7), 86.4 (C-1′), 80.4 (C-4′), 74.1 (C-2′), 71.9 (C-3′), 64.2 (C-5′), 44.4 (CH(CH2)5), 31.8, 31.8, 26.7, 26.7, 26.2 (5×CH2); HRMS (ESI-TOF) m/z: calcd for C38H35N3O7 ([M+H]+), 646.2547, found 646.2576.

Example 25. Synthesis of 2′,3′,5′-Tri-O-benzoyl-6-(4-isoproylphenyl)-9-β-D-ribofuranosyl-7-deazapurine (10m). Following the general procedure, compound 10m was obtained starting from 2′,3′,5′-tri-O-benzoyl-6-chloro-9-β-D-ribofuranosyl-7-desazapurine (9) (110 mg, 0.183 mmol), Fe(acac)3 (6.5 mg, 0.0183 mmol), CuI (7 mg, 0.026 mol), 4-isopropylphenylmagnesium bromide 0.5 M in THF (268.08 mg, 1.2 mmol, added as portions and TLC monitor) in dry THF (5.0 mL) and NMP (0.5 mL) after column chromatography on silica gel (Heptane/EtOAc=5:1, to 2:1, v/v) as white foam (100 mg, 35% yield). 1H NMR (300 MHz, CDCl3) δ 8.95 (s, 1H, H-2), 8.14 (d, J=7.9 Hz, 2H, Ph-H), 8.02-7.93 (m, 5H, Ph-H), 7.59-7.35 (m, 13H, H-8, Ph-H), 6.84 (d, J7,8=3.5 Hz, 1H, H-7), 6.84 (d, J=5.6 Hz, 1H, H-1′), 6.30 (dd, J2′,1′=5.6 Hz, J2′,3′=5.0 Hz, 1H, H-2′), 6.18 (dd, J3′,2′=5.0 Hz, J3′,4′=4.2 Hz, 1H, H-3′), 4.89 (dd, J5′,4′=3.0 Hz, Jgem=11.9 Hz, 1H, H-5′), 4.81 (ddd, J4′,3′=4.3 Hz, J4′,5′=3.2 Hz, J4′,5″=3.7 Hz, 1H, H-4′), 4.69 (dd, J5″,4′=3.7 Hz, Jgem=11.9 Hz, 1H, H-5″), 3.05-2.94 (m, 1H, CH(CH3)2), 1.30 (d, J=7.1 Hz, 6H, CH(CH3)2); 13C{1H} NMR (75 MHz, CDCl3) δ 166.4 (COOPh), 165.7 (COOPh), 165.4 (COOPh), 158.4 (C-6), 152.6 (C-4), 152.2 (C-2), 151.6 (C-Ph), 133.9 (C-Ph), 133.6 (C-Ph), 130.1 (C-Ph), 130.0 (C-Ph), 129.8 (C-Ph), 129.2 (C-Ph), 128.9 (C-Ph), 128.8 (C-Ph), 128.7 (C-Ph), 127.2 (C-Ph), 126.1 (C-8), 116. (C-5), 103.0 (C-7), 86.4 (C-1′), 80.4 (C-4′), 74.2 (C-2′), 71.9 (C-3′), 64.2 (C-5′), 34.8 (CH(CH3)2), 24.1, 24.1 (CH(CH3)2); HRMS (ESI-TOF) m/z: calcd for C41H35N3O7 ([M+H]+), 682.2547, found 682.2553.

Example 26. Synthesis of 2′,3′,5′-Tri-O-benzoyl-6-(4-methoxylphenyl)-9-β-D-ribofuranosyl-7-desazapurine (10n). Following the general procedure, compound 10n was obtained starting from 2′,3′,5′-tri-O-benzoyl-6-chloro-9-β-D-ribofuranosyl-7-desazapurine (9) (120 mg, 0.2 mmol), Fe(acac)3 (7 mg, 0.02 mmol), CuI (8 mg, 0.04 mol), 4-methoxylphenylmagnesium bromide 1 M in THF (158.51 mg, 0.75 mmol, added as portions and TLC monitor) in dry THF (5.0 mL) and NMP (0.5 mL) after column chromatography on silica gel (Heptane/EtOAc=5:1, to 2:1, v/v) as white foam (80 mg, 60% yield). 1H NMR (300 MHz, CDCl3) δ 8.91 (s, 1H, H-2), 8.17-8.92 (m, 8H, Ph-H), 7.60-7.35 (m, 9H, H-8, Ph-H), 7.05 (d, J=8.7 Hz, 2H, Ph-H), 6.83 (d, J7,8=3.6 Hz, 1H, H-7), 6.80 (d, J1′,2′=5.6 Hz, 1H, H-1′), 6.28 (dd, J2′,1′=5.6 Hz, J2′,3′=5.0 Hz, 1H, H-2′), 6.17 (dd, J3′,2′=5.0 Hz, J3′,4′=4.3 Hz, 1H, H-3′), 4.88 (dd, J5′,4′=3.0 Hz, Jgem=11.9 Hz, 1H, H-5′), 4.80 (ddd, J4′,3′=4.3 Hz, J4′,5′=3.0 Hz, J4′,5″=3.9 Hz, 1H, H-4′), 4.69 (dd, J5″,4′=3.8 Hz, Jgem=11.9 Hz, 1H, H-5″), 3.88 (s, 3H, OCH3); 13C{1H} NMR (75 MHz, CDCl3) δ 166.4 (COOPh), 165.7 (COOPh), 165.4 (COOPh), 161.7 (C-6), 157.9 (C-Ph), 152.6 (C-4), 152.2 (C-2), 133.8 (C-Ph), 133.6 (C-Ph), 130.8, (C-Ph), 130.6 (C-Ph), 130.1 (C-Ph), 129.8 (C-Ph), 129.2 (C-Ph), 128.9 (C-Ph), 128.8 (C-Ph), 128.7 (C-Ph), 125.9 (C-8), 116.3 (C-5), 102.9 (C-7), 86.5 (C-1′), 80.5 (C-4′), 74.2 (C-2′), 71.9 (C-3′), 64.2 (C-5′), 55.6 (OCH3); HRMS (ESI-TOF) m/z: calcd for C39H31N3O8 ([M+H]+), 670.2183, found 670.2195.

Example 27. Synthesis of 2′,3′,5′-Tri-O-benzoyl-6-phenyl-9-β-D-ribofuranosyl-7-deazapurine (100). Following the general procedure, compound 10o was obtained starting from 2′,3′,5′-tri-O-benzoyl-6-chloro-9-β-D-ribofuranosyl-7-desazapurine (9) (70 mg, 0.117 mmol), Fe(acac)3 (4 mg, 0.0117 mmol), CuI (5 mg, 0.02 mol), phenylmagnesium bromide 1 M in THF (54.39 mg, 0.3 mmol, added as portions and TLC monitor) in dry THF (5.0 mL) and NMP (0.5 mL) after column chromatography on silica gel (Heptane/EtOAc=5:1, to 2:1, v/v) as white foam (40 mg, 54% yield). 1H NMR (300 MHz, CDCl3) δ 8.96 (s, 1H, H-2), 8.15-8.79 (m, 8H, Ph-H), 7.59-7.32 (m, 13H, H-8, Ph-H), 6.83 (d, J7,8=3.6 Hz, 1H, H-7), 6.82 (d, J=5.6 Hz, 1H, H-1′), 6.29 (dd, J2′,1′=5.6 Hz, J2′,3′=5.3 Hz, 1H, H-2′), 6.17 (dd, J3′,2′=5.3 Hz, J3′,4′=4.4 Hz, 1H, H-3′), 4.89 (dd, J5′,4′=3.1 Hz, Jgem=11.9 Hz, 1H, H-5′), 4.79 (ddd, J4′,3′=4.4 Hz, J4′,5′=3.1 Hz, J4′,5″=3.5 Hz, 1H, H-4′), 4.70 (dd, J5″,4′=3.5 Hz, Jgem=11.9 Hz, 1H, H-5″); 13C{1H} NMR (75 MHz, CDCl3) δ 166.4 (COOPh), 165.7 (COOPh), 165.4 (COOPh), 158.4 (C-6), 152.7 (C-4), 152.2 (C-2), 138.2 (C-Ph), 133.9 (C-Ph), 133.6 (C-Ph), 130.4 (C-Ph), 130.0 (C-Ph), 129.8 (C-Ph), 129.1 (C-Ph), 129.0 (C-Ph), 128.8 (C-Ph), 128.7 (C-Ph), 126.4 (C-8), 116.9 (C-5), 102.8 (C-7), 86.7 (C-1′), 80.5 (C-4′), 74.2 (C-2′), 71.9 (C-3′), 64.2 (C-5′); HRMS (ESI-TOF) m/z: calcd for C38H29N3O7 ([M+H]+), 640.2078, found 640.2086.

Example 28. Synthesis of 2′,3′,5′-Tri-O-benzoyl-6-(4-(dimethylamino)phenyl)-9-β-D-ribofuranosyl-7-deazapurine (10p). Following the general procedure, compound 10p was obtained starting from 2′,3′,5′-tri-O-benzoyl-6-chloro-9-β-D-ribofuranosyl-7-desazapurine (9) (100 mg, 0.167 mmol), Fe(acac)3 (6 mg, 0.0167 mmol), CuI (6 mg, 0.032 mol), 4-(dimethylamino)phenylmagnesium bromide 0.5 M in THF (235.60 mg, 1.05 mmol, added as portions and TLC monitor) in dry THF (5.0 mL) and NMP (0.5 mL) after column chromatography on silica gel (Heptane/EtOAc=5:1, to 2:1, v/v) as light yellow foam (25 mg, 22% yield). 1H NMR (300 MHz, CDCl3) δ 8.87 (s, 1H, H-2), 8.15-8.79 (m, 8H, Ph-H), 7.59-7.32 (m, 11H, Ph-H), 6.86 (d, J8,7=3.7 Hz, 1H, H-8), 6.83 (d, J7,8=3.7 Hz, 1H, H-7), 6.80 (d, J=5.6 Hz, 1H, H-1′), 6.27 (dd, J2′,1′=5.6 Hz, J2′,3′=5.3 Hz, 1H, H-2′), 6.17 (dd, J3′,2′=5.3 Hz, J3′,4′=4.4 Hz, 1H, H-3′), 4.89 (dd, J5′,4′=2.9 Hz, Jgem=11.9 Hz, 1H, H-5′), 4.79 (ddd, J4′,3′=4.4 Hz, J4′,5′=2.9 Hz, J4′,5″=3.8 Hz, 1H, H-4′), 4.70 (dd, J5″,4′=3.8 Hz, Jgem=11.9 Hz, 1H, H-5″), 3.01 (s, 6H, 2×CH3); 13C{1H} NMR (75 MHz, CDCl3) δ 166.4 (COOPh), 165.7 (COOPh), 165.4 (COOPh), 158.4 (C-6), 152.6 (C-4), 152.2 (C-2), 152.1 (C-Ph), 133.8 (C-Ph), 133.6 (C-Ph), 130.4 (C-Ph), 130.1 (C-Ph), 130.0 (C-Ph), 129.8 (C-Ph), 129.1 (C-Ph), 128.8 (C-Ph), 128.7 (C-Ph), 125.7 130.1 (C-Ph), 125.1 (C-8), 115.7 (C-Ph), 112.1 (C-5), 103.3 (C-7), 86.7 (C-1′), 80.4 (C-4′), 74.2 (C-2′), 71.9 (C-3′), 64.3 (C-5′), 40.4 (2×CH3); HRMS (ESI-TOF) m/z: calcd for C40H34N4O7 ([M+H]+), 683.2500, found 683.2499.

Example 29. Synthesis of 2′,3′,5′-Tri-O-benzoyl-6-(4-ethylphenyl)-9-β-D-ribofuranosyl-7-deazapurine (10q). Following the general procedure, compound 10q was obtained starting from 2′,3′,5′-tri-O-benzoyl-6-chloro-9-β-D-ribofuranosyl-7-desazapurine (9) (120 mg, 0.2 mmol), Fe(acac)3 (7 mg, 0.02 mmol), CuI (8 mg, 0.04 mol), 4-ethylphenyl magnesium bromide 0.5 M in THF (345.46 mg, 1.65 mmol, added as portions and TLC monitor) in dry THF (5.0 mL) and NMP (0.5 mL) after column chromatography on silica gel (Heptane/EtOAc=5:1, to 2:1, v/v) as white foam (75 mg, 56% yield). 1H NMR (300 MHz, CDCl3) δ 8.94 (s, 1H, H-2), 8.14 (d, J=7.8 Hz, 2H, Ph-H), 8.02-7.93 (m, 5H, Ph-H), 7.59-7.35 (m, 13H, H-8, Ph-H), 6.84 (d, J=5.8 Hz, 1H, H-1′), 6.82 (d, J7,8=3.7 Hz, 1H, H-7), 6.29 (dd, J2′,1′=5.8 Hz, J2′,3′=5.0 Hz, 1H, H-2′), 6.18 (dd, J3′,2′=5.0 Hz, J3′,4′=4.3 Hz, 1H, H-3′), 4.89 (dd, J5′,4′=3.2 Hz, Jgem=11.9 Hz, 1H, H-5′), 4.81 (ddd, J4′,3′=4.3 Hz, J4′,5′=3.2 Hz, J4′,5″=3.8 Hz, 1H, H-4′), 4.69 (dd, J5″,4′=3.8 Hz, Jgem=11.9 Hz, 1H, H-5″), 2.74 (q, J=7.4 Hz, 2H, CH2), 1.29 (t, J=7.4 Hz, 3H, CH3); 13C{1H} NMR (75 MHz, CDCl3) δ 166.4 (COOPh), 165.7 (COOPh), 165.4 (COOPh), 158.4 (C-6), 152.6 (C-4), 152.2 (C-2), 147.0 (C-Ph), 133.9 (C-Ph), 133.6 (C-Ph), 130.1 (C-Ph), 130.0 (C-Ph), 129.8 (C-Ph), 129.1 (C-Ph), 128.8 (C-Ph), 128.7 (C-Ph), 128.6 (C-Ph), 126.1 (C-8), 116.7 (C-5), 103.2 (C-7), 86.4 (C-1′), 80.4 (C-4′), 74.2 (C-2′), 71.9 (C-3′), 64.2 (C-5′), 29.0 (CH2), 26.2 (CH3); HRMS (ESI-TOF) m/z: calcd for C40H33N3O7 ([M+H]+), 668.2391, found 668.2390.

Example 30. Synthesis of 6-(4-Methylphenyl)-9-β-D-ribofuranosyl-7-deazapurine (11a). Compound 10a (70 mg, 0.107 mmol) was dissolved in 20 ml 7 N NH3 in MeOH (2.38 g, 14.0 mmol) and the reaction mixture was stirred at room temperature overnight in a sealed vessel. It was then concentrated under reduced pressure and the resulting crude residue was purified by column chromatography on silica gel (gradient CH2Cl2/MeOH/, 10:1, v/v) to give 11a (30 mg, 83%) as a white solid. 1H NMR (300 MHz, DMSO-d6) δ 8.87 (s, 1H, H-2), 8.07 (d, J=8.0 Hz, 2H, Ph-H), 7.95 (d, J=3.8 Hz, 1H, H-8), 7.40 (d, J=8.0 Hz, 2H, Ph-H), 7.00 (d, J=3.8 Hz, 1H, H-7), 6.29 (d, J=6.1 Hz, 1H, H-1′), 5.41 (d, JOH,2′=6.3 Hz, 1H, OH-2′), 5.22 (d, JOH,3′=4.5 Hz, 1H, OH-3′), 5.13 (dd, JOH,5′=5.6 Hz, JOH,5″=4.4 Hz, 1H, OH-5′), 4.47 (ddd, J2′,1′=6.1 Hz, J2′,3′=4.7 Hz, J2′,OH=6.3 Hz, 1H, H-2′), 4.15 (ddd, J3′,2′=4.7 Hz, J3′,4′=3.7 Hz, J3′,OH=4.5 Hz, 1H, H-3′), 3.96 (ddd, J4′,3′=3.7 Hz, J4′,5′=4.5 Hz, J4′,5″=3.7 Hz, 1H, H-4′), 3.70-3.63 (ddd, J5′,4′=4.5 Hz, J5′,OH=5.6 Hz, Jgem=11.9 Hz, 1H, H-5′), 3.61-3.54 (ddd, J5″,4′=3.7 Hz, J5″,OH=4.4 Hz, Jgem=11.9 Hz, 1H, H-5″), 2.40 (s, 3H, CH3); 13C{1H} NMR (75 MHz, DMSO-d6) δ 156.2 (C-6), 152.0 (C-4), 151.0 (C-2), 140.2 (C-Ph), 134.9 (C-Ph), 129.6 (C-Ph), 128.6 (C-Ph), 127.9 (C-8), 115.3 (C-5), 101.1 (C-7), 87.0 (C-1′), 85.3 (C-4′), 74.2 (C-2′), 70.7 (C-3′), 61.7 (C-5′), 21.1 (CH3); HRMS (ESI-TOF) m/z: calcd for C17H19N3O4 ([M+H]+), 342.1448, found 342.1447.

Example 31. Synthesis of 6-Methyl-9-β-D-ribofuranosyl-7-deazapurine (11b). Following a similar procedure as that used for the synthesis of 11a, compound 11b was obtained starting from 10b (70 mg, 0.255 mmol) and 20 ml 7 N NH3 in MeOH (2.38 g, 14.0 mmol) after column chromatography on silica gel (CH2Cl2/MeOH=10:1, v/v) as white solid (25 mg, 78%). 1H NMR (300 MHz, DMSO-d6) δ 8.65 (s, 1H, H-2), 7.78 (d, J=3.8 Hz, 1H, H-8), 6.75 (d, J=3.8 Hz, 1H, H-7), 6.18 (d, J=6.1 Hz, 1H, H-1′), 5.35 (d, JOH,2′=6.4 Hz, 1H, OH-2′), 5.18 (d, JOH,3′=4.7 Hz, 1H, OH-3′), 5.10 (dd, JOH,5′=5.9 Hz, JOH,5″=4.9 Hz, 1H, OH-5′), 4.42 (ddd, J2′,1′=6.1 Hz, J2′,3′=5.6 Hz, J2′,OH=6.4 Hz, 1H, H-2′), 4.11 (ddd, J3′,2′=5.6 Hz, J3′,4′=3.8 Hz, J3′,OH=4.7 Hz, 1H, H-3′), 3.92 (ddd, J4′,3′=3.8 Hz, J4′,5′=3.9 Hz, J4′,5″=3.3 Hz, 1H, H-4′), 3.68-3.61 (ddd, J5′,4′=3.9 Hz, J5′,OH=5.9 Hz, Jgem=11.9 Hz, 1H, H-5′), 3.60-3.54 (ddd, J5″,4′=3.1 Hz, J5″,OH=4.9 Hz, Jgem=11.9 Hz, 1H, H-5″), 2.65 (s, 3H, CH3); 13C{1H} NMR (75 MHz, DMSO-d6) δ 159.0 (C-6), 150.8 (C-4), 150.4 (C-2), 126.5 (C-8), 118.0 (C-5), 100.1 (C-7), 87.1 (C-1′), 85.2 (C-4′), 74.1 (C-2′), 70.7 (C-3′), 61.7 (C-5′), 21.2 (CH3); HRMS (ESI-TOF) m/z: calcd for C12H15N3O4 ([M+H]+), 266.1135, found 266.1133.

Example 32. Synthesis of 6-Isopropyl-9-β-D-ribofuranosyl-7-deazapurine (11c). Following a similar procedure as that used for the synthesis of 11a, compound 11c was obtained starting from 10c (100 mg, 0.255 mmol) and 20 ml 7 N NH3 in MeOH (2.38 g, 14.0 mmol) after column chromatography on silica gel (CH2Cl2/MeOH=10:1, v/v) as white solid (40 mg, 83%). 1H NMR (300 MHz, DMSO-d6) δ 8.72 (s, 1H, H-2), 7.78 (d, J=3.7 Hz, 1H, H-8), 6.80 (d, J=3.7 Hz, 1H, H-7), 6.20 (d, J=6.3 Hz, 1H, H-1′), 5.37 (br, 1H, OH-2′), 5.19 (br, 1H, OH-3′), 5.11 (dd, JOH,5′=5.7 Hz, JOH,5″=4.6 Hz, 1H, OH-5′), 4.47 (dd, J2′,1′=6.1 Hz, J2′,3′=5.6 Hz, 1H, H-2′), 4.14 (dd, J3′,2′=5.6 Hz, J3′,4′=4.1 Hz, 1H, H-3′), 3.94 (ddd, J4′,3′=4.1 Hz, J4′,5′=3.9 Hz, J4′,5″=3.3 Hz, 1H, H-4′), 3.69-3.62 (ddd, J5′,4′=3.9 Hz, J5′,OH=5.7 Hz, Jgem=11.9 Hz, 1H, H-5′), 3.60-3.54 (ddd, J5″,4′=3.1 Hz, J5″,OH=4.6 Hz, Jgem=11.9 Hz, 1H, H-5″), 3.45-3.40 (m, 1H, CH(CH3)2), 1.31 (d, J=6.9 Hz, 6H, 2×CH3); 13C{1H} NMR (75 MHz, DMSO-d6) δ 167.1 (C-6), 151.0 (C-4), 150.8 (C-2), 126.6 (C-8), 116.4 (C-5), 99.8 (C-7), 86.9 (C-1′), 85.2 (C-4′), 74.0 (C-2′), 70.7 (C-3′), 61.7 (C-5′), 33.0 (CH(CH3)2), 21.5, 21.5 (CH(CH3)2); HRMS (ESI-TOF) m/z: calcd for C14H19N3O4 ([M+H]+), 294.1448, found 294.1447.

Example 32. Synthesis of 6-Ethyl-9-β-D-ribofuranosyl-7-deazapurine (11d). Following a similar procedure as that used for the synthesis of 11a, compound 11d was obtained starting from 10d (100 mg, 0.169 mmol) and 20 ml 7 N NH3 in MeOH (2.38 g, 14.0 mmol) after column chromatography on silica gel (CH2Cl2/MeOH=10:1, v/v) as white solid (40 mg, 85%). 1H NMR (300 MHz, DMSO-d6) δ 8.69 (s, 1H, H-2), 7.79 (d, J=3.8 Hz, 1H, H-8), 6.77 (d, J=3.8 Hz, 1H, H-7), 6.19 (d, J=6.1 Hz, 1H, H-1′), 5.36 (d, JOH,2′=6.4 Hz, 1H, OH-2′), 5.18 (d, JOH,3′=4.8 Hz, 1H, OH-3′), 5.10 (dd, JOH,5′=5.9 Hz, JOH,5″=5.1 Hz, 1H, OH-5′), 4.45 (ddd, J2′,1′=6.1 Hz, J2′,3′=5.6 Hz, J2′,OH=6.4 Hz, 1H, H-2′), 4.12 (ddd, J3′,2′=5.6 Hz, J3′,4′=4.1 Hz, J3′,OH=4.8 Hz, 1H, H-3′), 3.92 (ddd, J4′,3′=4.1 Hz, J4′,5′=3.9 Hz, J4′,5″=3.3 Hz, 1H, H-4′), 3.68-3.61 (ddd, J5′,4′=3.9 Hz, J5′,OH=5.9 Hz, Jgem=11.9 Hz, 1H, H-5′), 3.58-3.51 (ddd, J5″,4′=3.1 Hz, J5″,OH=5.0 Hz, Jgem=11.9 Hz, 1H, H-5″), 2.99 (q, J=7.7 Hz, 2H, CH2CH3), 1.30 (t, J=7.7 Hz, 3H, CH2CH3); 13C{1H} NMR (75 MHz, DMSO-d6) δ 163.6 (C-6), 151.0 (C-2), 150.6 (C-4), 126.6 (C-8), 117.2 (C-5), 99.9 (C-7), 87.0 (C-1′), 85.2 (C-2′), 74.0 (C-4′), 70.7 (C-3′), 61.7 (C-5′), 27.8 (CH2CH3), 12.7 (CH2CH3); HRMS (ESI-TOF) m/z: calcd for C15H19N3O4 ([M+H]+), 280.1291, found 280.1291.

Example 33. Synthesis of 6-Propyl-9-β-D-ribofuranosyl-7-deazapurine (11e). Following a similar procedure as that used for the synthesis of 11a, compound 11e was obtained starting from 10e (64 mg, 0.146 mmol) and 20 ml 7 N NH3 in MeOH (2.38 g, 14.0 mmol) after column chromatography on silica gel (CH2Cl2/MeOH=10:1, v/v) as white solid (25 mg, 81%). 1H NMR (300 MHz, DMSO-d6) δ 8.69 (s, 1H, H-2), 7.79 (d, J=3.7 Hz, 1H, H-8), 6.77 (d, J=3.7 Hz, 1H, H-7), 6.19 (d, J=6.2 Hz, 1H, H-1′), 5.37 (d, JOH,2′=6.4 Hz, 1H, OH-2′), 5.18 (d, JOH,3′=4.8 Hz, 1H, OH-3′), 5.10 (dd, JOH,5′=5.8 Hz, JOH,5″=5.0 Hz, 1H, OH-5′), 4.45 (ddd, J2′,1′=6.2 Hz, J2′,3′=5.4 Hz, J2′,OH=6.4 Hz, 1H, H-2′), 4.12 (ddd, J3′,2′=5.4 Hz, J3′,4′=4.3 Hz, J3′,OH=4.8 Hz, 1H, H-3′), 3.93 (ddd, J4′,3′=4.3 Hz, J4′,5′=3.5 Hz, J4′,5″=3.3 Hz, 1H, H-4′), 3.68-3.61 (ddd, J5′,4′=3.5 Hz, J5′,OH=5.8 Hz, Jgem=11.9 Hz, 1H, H-5′), 3.59-3.52 (ddd, J5″,4′=3.3 Hz, J5″,OH=5.0 Hz, Jgem=11.9 Hz, 1H, H-5″), 2.94 (t, J=7.3 Hz, 2H, CH2CH2), 1.85-1.73 (m, 2H, CH2CH3), 0.92 (t, J=7.3 Hz, 3H, CH2CH3), 13C{1H} NMR (75 MHz, DMSO-d6) δ 162.4 (C-6), 150.9 (C-2), 150.6 (C-4), 126.6 (C-8), 117.8 (C-5), 100.0 (C-7), 87.0 (C-1′), 85.2 (C-2′), 74.0 (C-4′), 70.7 (C-3′), 61.7 (C-5′), 36.5 (CH2CH2), 21.5 (CH2CH2), 13.9 (CH2CH3); HRMS (ESI-TOF) m/z: calcd for C15H19N3O4 ([M+H]+), 294.1448, found 294.1446.

Example 34. Synthesis of 6-Pentyl-9-β-D-ribofuranosyl-7-deazapurine (11f). Following a similar procedure as that used for the synthesis of 11a, compound 11f was obtained starting from 10f (80 mg, 0.126 mmol) and 20 ml 7 N NH3 in MeOH (2.38 g, 14.0 mmol) after column chromatography on silica gel (CH2Cl2/MeOH=10:1, v/v) as white semi-solid (35 mg, 87%). 1H NMR (300 MHz, DMSO-d6) δ 8.68 (s, 1H, H-2), 7.78 (d, J=3.8 Hz, 1H, H-8), 6.76 (d, J=3.8 Hz, 1H, H-7), 6.18 (d, J=6.3 Hz, 1H, H-1′), 5.35 (d, JOH,2′=6.4 Hz, 1H, OH-2′), 5.18 (d, JOH,3′=4.6 Hz, 1H, OH-3′), 5.09 (dd, JOH,5′=5.8 Hz, JOH,5″=5.0 Hz, 1H, OH-5′), 4.43 (ddd, J2′,1′=6.1 Hz, J2′,3′=5.6 Hz, J2′,OH=6.4 Hz, 1H, H-2′), 4.12 (ddd, J3′,2′=5.6 Hz, J3′,4′=4.1 Hz, J3′,OH=4.6 Hz, 1H, H-3′), 3.92 (ddd, J4′,3′=4.1 Hz, J4′,5′=3.7 Hz, J4′,5″=3.1 Hz, 1H, H-4′), 3.67-3.60 (ddd, J5′,4′=3.7 Hz, J5′,OH=5.8 Hz, Jgem=11.9 Hz, 1H, H-5′), 3.58-3.51 (ddd, J5″,4′=3.1 Hz, J5″,OH=5.0 Hz, Jgem=11.9 Hz, 1H, H-5″), 2.96 (t, J=7.4 Hz, 2H, CH2(CH2)3), 1.82-1.74 (m, 2H, CH2CH3)), 1.32-1.29 (m, 4H, CH2(CH2)2), 0.85 (t, J=6.6 Hz, 3H, (CH2)4CH3); 13C{1H} NMR (75 MHz, DMSO-d6) δ 162.7 (C-6), 150.9 (C-4), 150.6 (C-2), 126.6 (C-8), 117.7 (C-5), 99.9 (C-7), 87.0 (C-1′), 85.2 (C-2′), 74.0 (C-4′), 70.7 (C-3′), 61.7 (C-5′), 34.4, 31.1, 27.8, 22.0, 13.9 (aliphatic chain); HRMS (ESI-TOF) m/z: calcd for C18H19N3O5 ([M+H]+), 322.1761, found 322.1762.

Example 35. Synthesis of 6-Hexyl-9-β-D-ribofuranosyl-7-deazapurine (11g). Following a similar procedure as that used for the synthesis of 11a, compound 11g was obtained starting from 10g (86 mg, 0.146 mmol) and 20 ml 7 N NH3 in MeOH (2.38 g, 14.0 mmol) after column chromatography on silica gel (CH2Cl2/MeOH=10:1, v/v) as white semi-solid (38 mg, 86%). 1H NMR (300 MHz, CD3OD) δ 8.65 (s, 1H, H-2), 7.71 (d, J=3.8 Hz, 1H, H-8), 6.75 (d, J=3.8 Hz, 1H, H-7), 6.23 (d, J=6.3 Hz, 1H, H-1′), 4.65 (dd, J2′,1′=6.1 Hz, J2′,3′=5.2 Hz, 1H, H-2′), 4.32 (dd, J3′,2′=5.2 Hz, J3′,4′=3.2 Hz, 1H, H-3′), 3.92 (ddd, J4′,3′=3.2 Hz, J4′,5′=3.3 Hz, J4′,5″=2.9 Hz, 1H, H-4′), 3.68-3.61 (dd, J5′,4′=3.3 Hz, Jgem=11.9 Hz, 1H, H-5′), 3.60-3.54 (dd, J5″,4′=2.9 Hz, Jgem=11.9 Hz, 1H, H-5″), 3.03 (t, J=7.5 Hz, 2H, CH2(CH2)3), 1.86-1.76 (m, 2H, CH2CH3), 1.41-1.28 (m, 6H, (CH2)3CH3), 0.89 (t, J=6.8 Hz, 3H, (CH2)4CH3); 13C{1H} NMR (75 MHz, CD3OD) δ 163.0 (C-6), 149.9 (C-2), 149.6 (C-4), 127.0 (C-8), 118.4 (C-5), 99.4 (C-7), 88.8 (C-1′), 85.3 (C-2′), 73.9 (C-4′), 70.7 (C-3′), 61.6 (C-5′), 34.1, 30.9, 28.4, 28.3, 21.8, 12.5 (aliphatic chain); HRMS (ESI-TOF) m/z: calcd for C17H25N3O4 ([M+H]+), 336.1917, found 336.1917.

Example 36. Synthesis of 6-(But-3-en-1-yl)-9-β-D-ribofuranosyl-7-desazapurine (11h). Following a similar procedure as that used for the synthesis of 11a, compound 11h was obtained starting from 10h (80 mg, 0.08 mmol) and 20 ml 7 N NH3 in MeOH (2.38 g, 14.0 mmol) after column chromatography on silica gel (CH2C12/MeOH=10:1, v/v) as white semi-solid (16 mg, 66%). 1H NMR (300 MHz, CD3OD) δ 8.67 (s, 1H, H-2), 7.73 (d, J=3.8 Hz, 1H, H-8), 6.78 (d, J=3.8 Hz, 1H, H-7), 6.23 (d, J=6.3 Hz, 1H, H-1′), 4.65 (dd, J2′,1′=6.1 Hz, J2′,3′=5.2 Hz, 1H, H-2′), 5.93-5.82 (m, 1H, CH═CH2), 5.06 (dd, J=1.5 and 3.3 Hz, 1H, CH═CH), 5.01 (dd, J=1.5 and 3.3 Hz, 1H, CH═CH), 4.64 (dd, J3′,2′=5.2 Hz, J3′,4′=3.1 Hz, 1H, H-3′), 4.32 (ddd, J4′,3′=3.1 Hz, J4′,5′=3.3 Hz, J4′,5″=2.9 Hz, 1H, H-4′), 3.89-3.84 (dd, J5′,4′=3.3 Hz, Jgem=11.9 Hz, 1H, H-5′), 3.79-3.74 (dd, J5″,4′=2.9 Hz, Jgem=11.9 Hz, 1H, H-5″), 3.14 (t, J=7.1 Hz, 2H, CH2), 2.62-2.55 (m, 2H, CH2); 13C{1H} NMR (75 MHz, CD3OD) δ 162.0 (C-6), 150.0 (C-4), 149.6 (C-2), 136.6 (CH═CH2), 127.0 (C-8), 118.4 (C-5), 114.2 (CH═CH2), 99.4 (C-7), 88.8 (C-1′), 85.3 (C-2′), 73.9 (C-4′), 70.7 (C-3′), 61.6 (C-5′), 33.6 (CH2), 32.1 (CH2); HRMS (ESI-TOF) m/z: calcd for C15H19N3O4 ([M+H]+), 306.1448, found 306.1446.

Example 37. Synthesis of 6-Isobutyl-9-β-D-ribofuranosyl-7-deazapurine (11i). Following a similar procedure as that used for the synthesis of 11a, compound 11i was obtained starting from 10i (80 mg, 0.146 mmol) and 20 ml 7 N NH3 in MeOH (2.38 g, 14.0 mmol) after column chromatography on silica gel (CH2Cl2/MeOH=10:1, v/v) as white semi-solid (35 mg, 85%). 1H NMR (300 MHz, DMSO-d6) δ 8.69 (s, 1H, H-2), 7.78 (d, J=3.8 Hz, 1H, H-8), 6.76 (d, J=3.8 Hz, 1H, H-7), 6.19 (d, J=6.3 Hz, 1H, H-1′), 5.36 (d, JOH,2′=6.4 Hz, 1H, OH-2′), 5.18 (d, JOH,3′=4.8 Hz, 1H, OH-3′), 5.09 (dd, JOH,5′=5.8 Hz, JOH,5″=5.0 Hz, 1H, OH-5′), 4.45 (ddd, J2′,1′=6.3 Hz, J2′,3′=5.6 Hz, J2′,OH=6.4 Hz, 1H, H-2′), 4.12 (ddd, J3′,2′=5.6 Hz, J3′,4′=4.1 Hz, J3′,OH=4.8 Hz, 1H, H-3′), 3.92 (ddd, J4′,3′=4.1 Hz, J4′,5′=3.9 Hz, J4′,5″=3.3 Hz, 1H, H-4′), 3.67-3.60 (ddd, J5′,4′=3.9 Hz, J5′,OH=5.8 Hz, Jgem=11.9 Hz, 1H, H-5′), 3.58-3.51 (ddd, J5″,4′=3.1 Hz, J5″,OH=5.0 Hz, Jgem=11.9 Hz, 1H, H-5″), 2.84 (d, J=7.1 Hz, 2H, CH2CH), 2.27-2.18 (m, 1H, CH(CH3)2), 0.91 (d, J=6.9 Hz, 6H, CH(CH3)2); 13C{1H} NMR (75 MHz, DMSO-d6) δ 161.9 (C-6), 150.9 (C-2), 150.6 (C-4), 126.6 (C-8), 118.3 (C-5), 100.1 (C-7), 96.9 (C-1′), 85.2 (C-2′), 74.0 (C-4′), 70.7 (C-3′), 61.7 (C-5′), 43.6 (CH2CH), 28.1 (CH(CH3)2), 22.6 (CH3), 22.5 (CH3); HRMS (ESI-TOF) m/z: calcd for C15H21N3O4 ([M+H]+), 308.1604, found 308.1607.

Example 38. Synthesis of 6-Cycloproyl-9-β-D-ribofuranosyl-7-deazapurine (11j). Following a similar procedure as that used for the synthesis of 11a, compound 11j was obtained starting from 10j (95 mg, 0.157 mmol) and 20 ml 7 N NH3 in MeOH (2.38 g, 14.0 mmol) after column chromatography on silica gel (CH2C12/MeOH=10:1, v/v) as white solid (40 mg, 88%). 1H NMR (300 MHz, DMSO-d6) δ 8.58 (s, 1H, H-2), 7.77 (d, J=3.8 Hz, 1H, H-8), 6.88 (d, J=3.8 Hz, 1H, H-7), 6.17 (d, J=6.1 Hz, 1H, H-1′), 5.34 (d, JOH,2′=6.4 Hz, 1H, OH-2′), 5.17 (d, JOH,3′=4.6 Hz, 1H, OH-3′), 5.11 (dd, JOH,5′=5.7 Hz, JOH,5″=4.6 Hz, 1H, OH-5′), 4.43 (dd, J2′,1′=6.1 Hz, J2′,3′=5.6 Hz, 1H, H-2′), 4.12 (dd, J3′,2′=5.6 Hz, J3′,4′=4.1 Hz, 1H, H-3′), 3.92 (ddd, J4′,3′=4.1 Hz, J4′,5′=3.9 Hz, J4′,5″=3.3 Hz, 1H, H-4′), 3.69-3.61 (ddd, J5′,4′=3.9 Hz, J5′,OH=5.7 Hz, Jgem=11.9 Hz, 1H, H-5′), 3.59-3.53 (ddd, J5″,4′=3.1 Hz, J5″,OH=4.6 Hz, Jgem=11.9 Hz, 1H, H-5″), 2.54-2.46 (m, 1H, CH), 1.18-1.10 (m, 4H, CH(CH2)2); 13C{1H} NMR (75 MHz, DMSO-d6) δ 163.6 (C-6), 151.1 (C-2), 149.9 (C-4), 126.4 (C-8), 117.4 (C-5), 99.8 (C-7), 87.0 (C-1′), 85.2 (C-2′), 74.0 (C-4′), 70.7 (C-3′), 61.7 (C-5′), 14.0 (CH(CH2)2), 10.7 (CH(CH2)2); HRMS (ESI-TOF) m/z: calcd for C14H17N3O4 ([M+H]+), 292.1291, found 292.1291.

Example 39. Synthesis of 6-Cyclopentyl-9-β-D-ribofuranosyl-7-deazapurine (11k). Following a similar procedure as that used for the synthesis of 11a, compound 11k was obtained starting from 10k (80 mg, 0.126 mmol) and 20 ml 7 N NH3 in MeOH (2.38 g, 14.0 mmol) after column chromatography on silica gel (CH2Cl2/MeOH=10:1, v/v) as white semi-solid (35 mg, 87%). 1H NMR (300 MHz, DMSO-d6) δ 8.70 (s, 1H, H-2), 8.07 (d, J=8.0 Hz, 2H, Ph-H), 7.77 (d, J=3.8 Hz, 1H, H-8), 6.77 (d, J=3.8 Hz, 1H, H-7), 6.19 (d, J=6.1 Hz, 1H, H-1′), 5.36 (d, JOH,2′=6.3 Hz, 1H, OH-2′), 5.19 (d, JOH,3′=4.7 Hz, 1H, OH-3′), 5.13 (dd, JOH,5′=5.7 Hz, JOH,5″=4.3 Hz, 1H, OH-5′), 4.45 (ddd, J2′,1′=6.1 Hz, J2′,3′=4.9 Hz, J2′,OH=6.3 Hz, 1H, H-2′), 4.13 (ddd, J3′,2′=4.9 Hz, J3′,4′=3.7 Hz, J3′,OH=4.7 Hz, 1H, H-3′), 3.93 (ddd, J4′,3′=3.7 Hz, J4′,5′=4.5 Hz, J4′,5″=3.7 Hz, 1H, H-4′), 3.67-3.52 (ddd, J5′,4′=4.5 Hz, J5′,OH=5.7 Hz, Jgem=11.9 Hz, 1H, H-5′), 3.59-3.52 (ddd, J5″,4′=3.7 Hz, J5″,OH=4.3 Hz, Jgem=11.9 Hz, 1H, H-5″), 2.09-1.62 (m, 8H); 13C{1H} NMR (75 MHz, DMSO-d6) δ 165.9 (C-6), 151.0 (C-2), 150.5 (C-4), 126.5 (C-8), 117.2 (C-5), 100.0 (C-7), 86.9 (C-1′), 85.2 (C-2′), 74.0 (C-4′), 70.7 (C-3′), 61.7 (C-5′), 43.9 (CH(CH2)4), 32.2, 32.1, 25.9, 25.9 (4×CH2); HRMS (ESI-TOF) m/z: calcd for C16H21N3O4 ([M+H]+), 320.1604, found 320.1607.

Example 40. Synthesis of 6-Cyclohexyl-9-β-D-ribofuranosyl-7-deazapurine (11l). Following a similar procedure as that used for the synthesis of 11a, compound 11l was obtained starting from 10l (80 mg, 0.146 mmol) and 20 ml 7 N NH3 in MeOH (2.38 g, 14.0 mmol) after column chromatography on silica gel (CH2C12/MeOH=10:1, v/v) as white semi-solid (35 mg, 85%). 1H NMR (300 MHz, DMSO-d6) δ 8.69 (s, 1H, H-2), 7.78 (d, J=3.8 Hz, 1H, H-8), 6.80 (d, J=3.8 Hz, 1H, H-7), 6.18 (d, J=6.3 Hz, 1H, H-1′), 5.34 (d, JOH,2′=6.4 Hz, 1H, OH-2′), 5.16 (d, JOH,3′=4.8 Hz, 1H, OH-3′), 5.09 (dd, JOH,5′=5.9 Hz, JOH,5″=4.8 Hz, 1H, OH-5′), 4.45 (ddd, J2′,1′=6.3 Hz, J2′,3′=5.6 Hz, J2′,OH=6.4 Hz, 1H, H-2′), 4.12 (ddd, J3′,2′=5.6 Hz, J3′,4′=4.1 Hz, J3′,OH=4.8 Hz, 1H, H-3′), 3.92 (ddd, J4′,3′=4.1 Hz, J4′,5′=3.9 Hz, J4′,5″=3.3 Hz, 1H, H-4′), 3.67-3.60 (ddd, J5′,4′=3.9 Hz, J5′,OH=5.9 Hz, Jgem=11.9 Hz, 1H, H-5′), 3.58-3.52 (ddd, J5″,4′=3.1 Hz, J5″,OH=5.0 Hz, Jgem=11.9 Hz, 1H, H-5″), 3.11 (t, J=11.4 Hz, 1H, CH(CH2)5), 1.84-1.64 (m, 7H), 1.50-1.23 (m, 3H); 13C{1H} NMR (75 MHz, DMSO-d6) δ 166.2 (C-6), 151.0 (C-2), 150.8 (C-4), 126.5 (C-8), 116.6 (C-5), 99.8 (C-7), 86.9 (C-1′), 85.2 (C-2′), 74.0 (C-4′), 70.7 (C-3′), 61.7 (C-5′), 43.0 (CH(CH2)5), 31.3, 31.3, 25.9, 25.7, 25.7 (5×CH2); HRMS (ESI-TOF) m/z: calcd for C17H23N3O4 ([M+H]+), 334.1761, found 334.1762.

Example 41. Synthesis of 6-(4-isoproylphenyl)-9-β-D-ribofuranosyl-7-deazapurine (11m). Following a similar procedure as that used for the synthesis of 11a, compound 11m was obtained starting from 10m (100 mg, 0.146 mmol) and 20 ml 7 N NH3 in MeOH (2.38 g, 14.0 mmol) after column chromatography on silica gel (CH2Cl2/MeOH=10:1, v/v) as white solid (40 mg, 74%). 1H NMR (300 MHz, DMSO-d6) δ 8.87 (s, 1H, H-2), 8.11 (d, J=8.2 Hz, 2H, Ph-H), 7.95 (d, J=3.8 Hz, 1H, H-8), 7.47 (d, J=8.2 Hz, 2H, Ph-H), 7.01 (d, J=3.8 Hz, 1H, H-7), 6.28 (d, J=6.1 Hz, 1H, H-1′), 5.40 (d, JOH,2′=6.3 Hz, 1H, OH-2′), 5.20 (d, JOH,3′=4.8 Hz, 1H, OH-3′), 5.11 (dd, JOH,5′=5.7 Hz, JOH,5″=4.8 Hz, 1H, OH-5′), 4.47 (ddd, J2′,1′=6.1 Hz, J2′,3′=5.6 Hz, J2′,OH=6.3 Hz, 1H, H-2′), 4.14 (ddd, J3′,2′=5.6 Hz, J3′,4′=4.1 Hz, J3′,OH=4.8 Hz, 1H, H-3′), 3.94 (ddd, J4′,3′=4.1 Hz, J4′,5′=3.9 Hz, J4′,5″=3.3 Hz, 1H, H-4′), 3.69-3.62 (ddd, J5′,4′=3.9 Hz, J5′,OH=5.7 Hz, Jgem=11.9 Hz, 1H, H-5′), 3.60-3.54 (ddd, J5″,4′=3.1 Hz, J5″,OH=4.6 Hz, Jgem=11.9 Hz, 1H, H-5″), 3.04-2.95 (m, 1H, CH(CH3)2), 1.27 (d, J=6.9 Hz, 6H, 2×CH3); 13C{1H} NMR (75 MHz, DMSO-d6) δ 156.2 (C-6), 152.1 (C-4), 151.1 (C-2), 150.9 (C-Ph), 135.3 (C-Ph), 128.8 (C-Ph), 127.9 (C-Ph), 126.9 (C-8), 115.4 (C-5), 101.0 (C-7), 87.0 (C-1′), 85.3 (C-4′), 74.2 (C-2′), 70.7 (C-3′), 61.7 (C-5′), 33.4 (CH(CH3)2), 23.8, 23.8 (CH(CH3)2); HRMS (ESI-TOF) m/z: calcd for C20H23N3O4 ([M+H]+), 370.1761, found 370.1755.

Example 42. Synthesis of 6-(4-Methoxylphenyl)-9-β-D-ribofuranosyl-7-desazapurine (11n). Following a similar procedure as that used for the synthesis of 11a, compound 11n was obtained starting from 10n (80 mg, 0.146 mmol) and 20 ml 7 N NH3 in MeOH (2.38 g, 14.0 mmol) after column chromatography on silica gel (CH2Cl2/MeOH=10:1, v/v) as white solid (35 mg, 84%). 1H NMR (300 MHz, DMSO-d6) δ 8.83 (s, 1H, H-2), 8.18 (d, J=8.8 Hz, 2H, Ph-H), 7.93 (d, J=3.8 Hz, 1H, H-8), 7.14 (d, J=8.8 Hz, 2H, Ph-H), 7.01 (d, J=3.8 Hz, 1H, H-7), 6.28 (d, J=6.1 Hz, 1H, H-1′), 5.40 (d, JOH,2′=6.3 Hz, 1H, OH-2′), 5.21 (d, JOH,3′=4.7 Hz, 1H, OH-3′), 5.11 (dd, JOH,5′=5.9 Hz, JOH,5″=4.8 Hz, 1H, OH-5′), 4.47 (ddd, J2′,1′=6.3 Hz, J2′,3′=5.6 Hz, J2′,OH=6.3 Hz, 1H, H-2′), 4.14 (ddd, J3′,2′=5.6 Hz, J3′,4′=4.1 Hz, J3′,OH=4.7 Hz, 1H, H-3′), 3.95 (ddd, J4′,3′=4.1 Hz, J4′,5′=3.9 Hz, J4′,5″=3.3 Hz, 1H, H-4′), 3.86 (s, 3H, CH3), 3.70-3.60 (ddd, J5′,4′=3.9 Hz, J5′,OH=5.9 Hz, Jgem=11.9 Hz, 1H, H-5′), 3.59-3.54 (ddd, J5″,4′=3.1 Hz, J5″,OH=5.0 Hz, Jgem=11.9 Hz, 1H, H-5″), 3.48 (s, 3H, —OCH3); 13C{1H} NMR (75 MHz, DMSO-d6) δ 161.1 (C-6), 155.8 (C-Ph), 152.0 (C-4), 151.0 (C-2), 130.3 (C-Ph), 130.0 (C-Ph), 127.7 (C-8), 114.9 (C-Ph), 114.4 (C-5), 101.1 (C-7), 86.8 (C-1′), 85.3 (C-4′), 74.1 (C-2′), 70.7 (C-3′), 61.7 (C-5′), 55.4 (OCH3); HRMS (ESI-TOF) m/z: calcd for C18H19N3O5 ([M+H]+), 358.1397, found 358.1391.

Example 43. Synthesis of 6-Phenyl-9-β-D-ribofuranosyl-7-deazapurine (110). Following a similar procedure as that used for the synthesis of 11a, compound 110 was obtained starting from 10o (80 mg, 0.146 mmol) and 20 ml 7 N NH3 in MeOH (2.38 g, 14.0 mmol) after column chromatography on silica gel (CH2Cl2/MeOH=10:1, v/v) as white solid (15 mg, 75%). 1H NMR (300 MHz, DMSO-d6) δ 8.90 (s, 1H, H-2), 8.17 (d, J=7.9 Hz, 2H, Ph-H), 7.97 (d, J=3.8 Hz, 1H, H-8), 7.61-7.57 (m, 3H), 7.01 (d, J=3.8 Hz, 1H, H-7), 6.29 (d, J=6.0 Hz, 1H, H-1′), 5.42 (d, JOH,2′=5.6 Hz, 1H, OH-2′), 5.22 (d, JOH,3′=4.4 Hz, 1H, OH-3′), 5.13 (dd, JOH,5′=5.6 Hz, JOH,5″=4.2 Hz, 1H, OH-5′), 4.47 (ddd, J2′,1′=6.1 Hz, J2′,3′=5.0 Hz, J2′,OH=5.6 Hz, 1H, H-2′), 4.14 (ddd, J3′,2′=5.0 Hz, J3′,4′=3.7 Hz, J3′,OH=4.4 Hz, 1H, H-3′), 3.93 (ddd, J4′,3′=3.7 Hz, J4′,5′=4.5 Hz, J4′,5″=3.7 Hz, 1H, H-4′), 3.69-3.62 (ddd, J5′,4′=4.5 Hz, J5′,OH=5.6 Hz, Jgem=11.9 Hz, 1H, H-5′), 3.61-3.51 (ddd, J5″,4′=3.7 Hz, J5″,OH=4.2 Hz, Jgem=11.9 Hz, 1H, H-5″); 13C{1H} NMR (75 MHz, DMSO-d6) δ 156.2 (C-6), 152.1 (C-4), 151.1 (C-2), 137.6 (C-Ph), 130.3 (C-Ph), 129.0 (C-Ph), 128.7 (C-Ph), 128.1 (C-8), 115.6 (C-5), 101.0 (C-7), 87.0 (C-1′), 85.3 (C-2′), 74.2 (C-4′), 70.7 (C-3′), 61.7 (C-5′); HRMS (ESI-TOF) m/z: calcd for C17H17N3O4 ([M+H]+), 328.1291, found 328.1296.

Example 44. Synthesis of 6-(4-(Dimethylamino)phenyl)-9-β-D-ribofuranosyl-7-deazapurine (11p). Following a similar procedure as that used for the synthesis of 11a, compound 11p was obtained starting from 10p (25 mg, 0.036 mmol) and 20 ml 7 N NH3 in MeOH (2.38 g, 14.0 mmol) after column chromatography on silica gel (CH2C12/MeOH=10:1, v/v) as white solid (11 mg, 85%). 1H NMR (300 MHz, DMSO-d6) δ 8.75 (s, 1H, H-2), 8.12 (d, J=9.0 Hz, 2H, Ph-H), 7.86 (d, J=3.8 Hz, 1H, H-8), 6.99 (d, J=3.8 Hz, 1H, H-8), 6.87 (d, J=9.0 Hz, 2H, Ph-H), 6.24 (d, J=6.1 Hz, 1H, H-1′), 5.38 (d, JOH,2′=6.3 Hz, 1H, OH-2′), 5.19 (d, JOH,3′=4.7 Hz, 1H, OH-3′), 5.13 (dd, JOH,5′=5.9 Hz, JOH,5″=4.8 Hz, 1H, OH-5′), 4.45 (ddd, J2′,1′=6.3 Hz, J2′,3′=5.6 Hz, J2′,OH=6.3 Hz, 1H, H-2′), 4.13 (ddd, J3′,2′=5.6 Hz, J3′,4′=4.1 Hz, J3′,OH=4.7 Hz, 1H, H-3′), 3.93 (ddd, J4′,3′=4.1 Hz, J4′,5′=3.9 Hz, J4′,5″=3.3 Hz, 1H, H-4′), 3.69-3.62 (ddd, J5′,4′=3.9 Hz, J5′,OH=5.9 Hz, Jgem=11.9 Hz, 1H, H-5′), 3.60-3.54 (ddd, J5″,4′=3.1 Hz, J5″,OH=5.0 Hz, Jgem=11.9 Hz, 1H, H-5″), 3.03 (s, 6H, 2×CH3); 13C{1H} NMR (75 MHz, DMSO-d6) δ 156.4 (C-6), 151.9 (C-4), 151.8 (C-Ph), 150.9 (C-2), 129.9 (C-Ph), 127.0 (C-Ph), 124.8 (C-8), 114.2 (C-Ph), 111.9 (C-5), 101.3 (C-7), 87.0 (C-1′), 85.2 (C-2′), 74.1 (C-4′), 70.7 (C-3′), 61.7 (C-5′), 39.8 (CH3); HRMS (ESI-TOF) m/z: calcd for C19H22N4O4 ([M+H]+), 371.1713, found 371.1708.

Example 45. Synthesis of 6-(4-Ethylphenyl)-9-β-D-ribofuranosyl-7-deazapurine (11q). Following a similar procedure as that used for the synthesis of 11a, compound 11q was obtained starting from 10q (75 mg, 0.112 mmol) and 20 ml 7 N NH3 in MeOH (2.38 g, 14.0 mmol) after column chromatography on silica gel (CH2C12/MeOH=10:1, v/v) as white solid (30 mg, 77%). 1H NMR (300 MHz, DMSO-d6) δ 8.87 (s, 1H, H-2), 8.10 (d, J=8.2 Hz, 2H, Ph-H), 7.95 (d, J=3.7 Hz, 1H, H-8), 7.44 (d, J=8.2 Hz, 2H, Ph-H), 7.01 (d, J=3.8 Hz, 1H, H-7), 6.28 (d, J=6.0 Hz, 1H, H-1′), 5.40 (d, JOH,2′=6.4 Hz, 1H, OH-2′), 5.20 (d, JOH,3′=4.8 Hz, 1H, OH-3′), 5.11 (dd, JOH,5′=5.7 Hz, JOH,5″=4.8 Hz, 1H, OH-5′), 4.47 (ddd, J2′,1′=6.0 Hz, J2′,3′=5.3 Hz, J2′,OH=6.4 Hz, 1H, H-2′), 4.14 (ddd, J3′,2′=5.3 Hz, J3′,4′=4.0 Hz, J3′,OH=4.8 Hz, 1H, H-3′), 3.94 (ddd, J4′,3′=4.0 Hz, J4′,5′=3.7 Hz, J4′,5″=3.1 Hz, 1H, H-4′), 3.70-3.63 (ddd, J5′,4′=3.7 Hz, J5′,OH=5.7 Hz, Jgem=11.9 Hz, 1H, H-5′), 3.61-3.53 (ddd, J5″,4′=3.1 Hz, J5″,OH=4.8 Hz, Jgem=11.9 Hz, 1H, H-5″), 2.71 (q, J=7.5 Hz, 1H, CH2CH3), 1.25 (t, J=7.5 Hz, 3H, CH2CH3); 13C{1H} NMR (75 MHz, DMSO-d6) δ 156.2 (C-6), 152.0 (C-4), 151.0 (C-2), 146.3 (C-Ph), 135.2 (C-Ph), 128.7 (C-Ph), 128.4 (C-Ph), 127.9 (C-8), 115.3 (C-5), 101.7 (C-7), 87.0 (C-1′), 85.3 (C-2′), 74.2 (C-4′), 70.7 (C-3′), 61.7 (C-5′), 28.1 (CH2CH3), 15.4 (CH2CH3); HRMS (ESI-TOF) m/z: calcd for C19H21N3O4 ([M+H]+), 356.1604, found 356.1590.

Example 46. Scale up experiment of compound 5h. An oven-dried flask was charged with 6-chloro-7-deazapurine (2.0 g, 13 mmol, 1 equiv) in THF 50 mL, NMP 5 mL, Fe(acac)3 (460 mg, 1.3 mmol, 0.1 equiv) and CuI (490 mg, 2.6 mmol, 0.2 equiv). The mixture was placed in an ice bath, and cyclohexyl magnesium bromide 1 M in THF (6.93 g, 37 mmol, added as portions slowly and TLC monitor) was added. The reaction mixture was stirred for 30 minutes in an ice bath. Monitoring with TLC till starting material disappeared, the reaction was quenched by the addition of aq. saturated solution of NH4Cl and extracted with EtOAc (3×100 mL). The organic solution was dried over Na2SO4 and concentrated under reduced pressure. The crude product was purified by silica gel chromatography (Heptane/EtOAc=5:1, to DCM:MeOH=30:1, to afford the desired product as pale white solid (2.2 g, 85%).

Example 47. Scale up experiment of compound 5k. An oven-dried flask was charged with 6-chloro-7-deazapurine (2.0 g, 13 mmol, 1 equiv) in THF 50 mL, NMP 5 mL, Fe(acac)3 (460 mg, 1.3 mmol, 0.1 equiv) and CuI (490 mg, 2.6 mmol, 0.2 equiv). The mixture was placed in an ice bath, and propyl magnesium chloride 2 M in THF (3.70 g, 36 mmol, added as portions slowly and TLC monitor) was added. The reaction mixture was stirred for 20 minutes in an ice bath. Monitoring with TLC till starting material disappeared, the reaction was quenched by the addition of aq. saturated solution of NH4Cl and extracted with EtOAc (3×100 mL). The organic solution was dried over Na2SO4 and concentrated under reduced pressure. The crude product was purified by silica gel chromatography (Heptane/EtOAc=5:1, to DCM MeOH=30:1, to afford the desired product as pale white solid (1.7 g, 81%).

Example 48. In-Vitro Antiviral Screening

The novel compounds described in the present application were screened against a number of different norovirus to determine their potential antiviral properties. The screening was performed as described previously (Stable expression of a Norwalk virus RNA replicon in a human hepatoma cell line—Kyeong-Ok Chang et al., Virology 353 (2006) 463-473). Briefly, the compounds were tested against a stable cell line expressing NV RNA; the cell line allows the study of virus and host interactions and provide a platform for screening anti-viral compounds.

### Results

The coupling reaction was successfully achieved in the presence of a catalytic system combining Fe catalyst [FeCl₃ or Fe(acac)₃] and copper(I) iodide under mild conditions to give the corresponding cross-coupling products in medium to good yields. This catalytic mixture offers an efficient alternative to the Pd- and Ni-catalyzed procedures often used until now.

TABLE 1
Fe-Catalyzed Cross-Coupling of 6-Chloro-7-Deazapurine (4) with
Phenylmagnesium Bromide^{a}

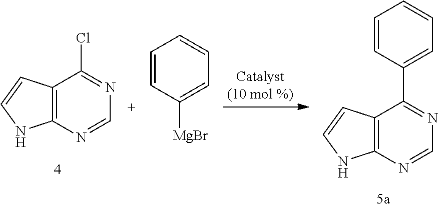

| Entry | Solvent | Catalyst | Yield |
|---|---|---|---|
| 1 | THF | FeCl₃ | 57% |
| 2 | THF | None | 0 |
| 3 | THF | ZnCl₂ | 0 |
| 4 | THF | CuCl₂ | 0 |
| 5 | THF | FeCl₃•6H₂O | 55% |
| 6 | THF/NMP (10:1) | FeCl₃ | 65% |

^{a}Reaction conditions: 4 (1 equiv), PhMgBr (5.00 equiv), catalyst (0.1 equiv), THF (5 mL) or THF/NMP (5 mL /0.5 mL), 0° C.-rt, 3 h, and isolated yields after silica gel chromatography.

Coupling of 6-chloro-7-deazapurine (4) with Phenylmagnesium Bromide Using FeCl₃ (10 mol %) as the Catalyst in THF as the Solvent at 0° C. to Room Temperature.

The desired product 5a was isolated in 57% yield. However, no desired product was obtained when the reaction was carried out without catalysts or in the presence of ZnCl₂ and CuCl₂. The yield (55%) to afford 5a by using FeCl₃.6H₂O was comparable to the yield obtained with FeCl₃. In the light of Fürstner's previous work²⁰ and recent mechanistic studies on addition of N-methylpyrrolidone (NMP) in Fe-catalyzed cross-coupling reaction²³, an increase in the yield was observed when using N-methylpyrrolidone (NMP) as the cosolvent in combination with FeCl₃ (Table 1, entry 6).

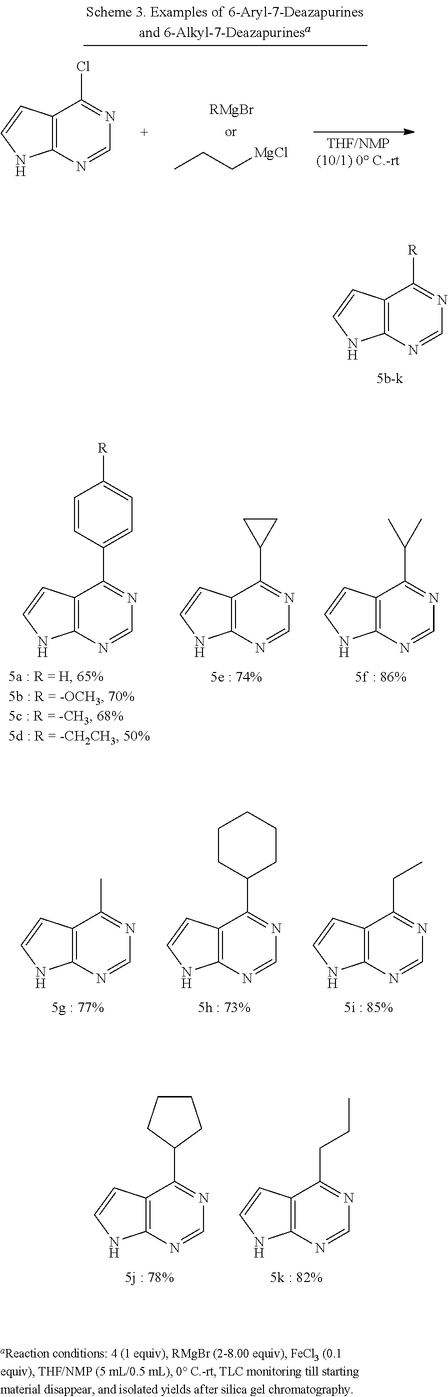

Once the reaction conditions were optimized, 6-chloro-7-deazapurine (4) was reacted with a series of aryl and alkyl Grignard reagents (Scheme 3). The results summarized in Scheme 3 show that the conditions described above proved to be useful for the coupling of 4 with a series of functionalized Grignard reagents. 4-Methoxy-, 4-methyl-, and 4-ethylphenylmagnesium bromide underwent reaction with 4 to give products 5b-d in 50-70% yields (Scheme 3). Apart of Csp²-Csp² bond formation, it was demonstrated that the reaction had a generic character by successfully carrying out Csp²-Csp³ bond formation (Scheme 3). Primary and secondary alkyl Grignard reagents also reacted well with 4 to give the coupling products 5e-k in good yields. Reaction of ethylmagnesium bromide with 4 without N-methylpyrrolidone (NMP) generated 5i in low yield, together with the 6-dechlorinated compound.

The same reaction was tested out for the synthesis of the corresponding nucleoside analogues. The starting material (compound 9) was obtained as shown in Scheme 4.

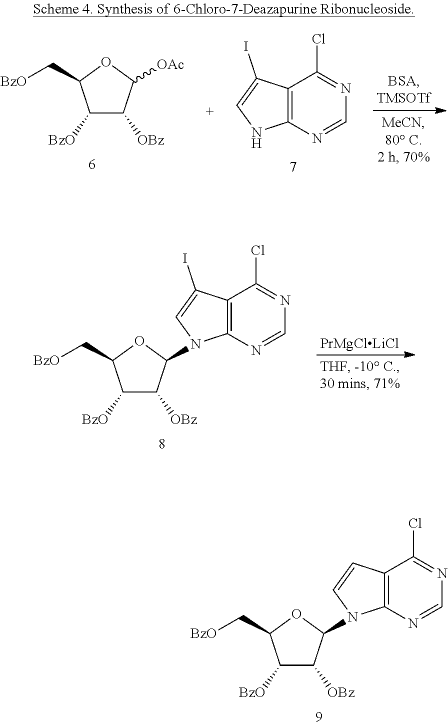

Compound 8 was obtained according to the literature reported by Seela, F. et al.²⁴ Deiodination of 8 was achieved by iodine-magnesium exchange reaction using Knochel's Turbo-Grignard reagent²⁵⁻²⁶ (iPrMgCl.LiCl) and subsequent hydrolysis of the magnesium intermediate to give 2′,3′,5′-tri-O-benzoyl-6-chloro-9-β-D-ribofuranosyl-7-desazapurine (9)²⁷ in 71% yield.

TABLE 2
Fe-catalyzed Cross-Coupling of Substrate 9 with 4-Methylphenyl-
magnesium Bromide^{a}

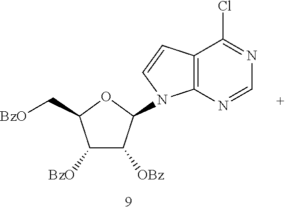

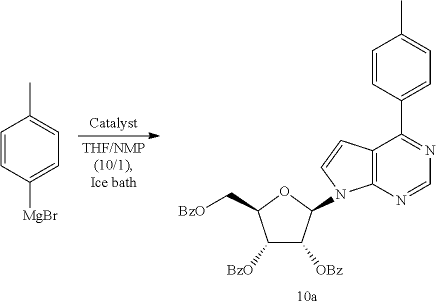

| Entry | Catalyst | Yield |
|---|---|---|
| 1 | FeCl₃ (10 mol %) | 42% |
| 2 | Fe(acac)₃ (10 mol %) | 55% |

^{a}Reaction conditions: 9 (1 equiv), PhMgBr (5.00 equiv), Fe catalyst (0.1 equiv), THF/NMP (5 mL /0.5 mL), 0° C.-rt, TLC monitor till starting material disappear, and isolated yields after silica gel chromatography. acac = acetylacetonate.

The coupling of 4-methylphenylmagnesium bromide with substrate 9 was used as a model reaction. FeCl₃ is less effective than Fe(acac)₃ as a catalyst in this case. Subsequently, Fe(acac)₃ was examined as a catalyst in the coupling reaction of substrate 9 with methyl magnesium bromide. The results revealed no significant improvement in yield (Table 3, entry 1). However, the reaction could be improved if an additive is included in the reaction system. Among the tested additives, CuI appears to be the better one, resulting in 10b and 10c in good yields (Table 3, entries 2 and 4). The use of organic zinc reagent or CuI as sole catalyst was not successful in the cross-coupling reaction between substrate 9 and isopropylmagnesium bromide (Table 3, entries 5 and 6).

TABLE 3
Evaluation of the Reaction Conditions^{a} for the Synthesis of 6-Alkyl 7-
Deazapurine Ribonucleosides

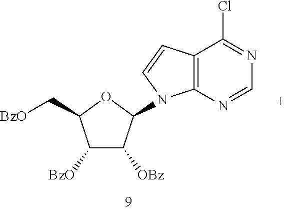

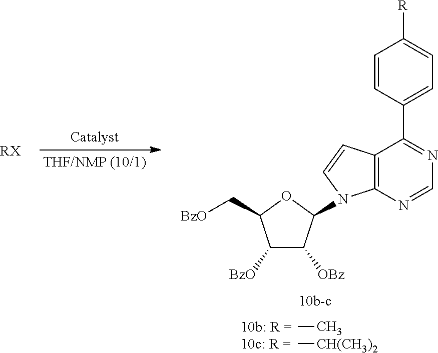

| Entry | RX | Catalyst | Yield |
|---|---|---|---|
| 1 | CH₃MgBr | Fe(acac)₃ (10 mol % | 41% |
| 2 | CH₃MgBr | Fe(acac)₃ (10 mol %)/ | 61% |
| | | Cul (20 mol %) | |
| 3 | (CH₃)₂CHMgBr | Fe(acac)₃ (10 mol %) | 57% |
| 4 | (CH₃)₂CHMgBr | Fe(acac)₃ (10 mol %)/ | 71% |
| | | Cul (20 mol %) | |
| 5 | (CH₃)₂CHZnCl | Fe(acac)₃ (10 mol %) | 0 |
| 6 | (CH₃)₂CHMgBr | Cul (10 mol %) | 0^{b} |

^{a}Reaction conditions: 9 (1 equiv), Metal complex (2.00 equiv), Fe catalyst (0.1 equiv), Cul (0.2 equiv), THF/NMP (5 mL /0.5 mL), ice bath, TLC monitoring, and isolated yields after silica gel chromatography. acac = acetylacetonate
^{b}Reaction with Cul alone gave no product.

Under the optimized reaction conditions, the substrate scope for the Fe/Cu-catalyzed coupling of structurally diverse Grignard reagents with substrate 9 was examined (Scheme 5). The results, summarized in Scheme 5, show that arylmagnesium bromide underwent reaction with 9 to give products 10m-q in medium to good yields (Scheme 5, 10m-q). The potential of this reaction for a Csp²-Csp³ bond formation (Scheme 5, 10b-l) was also examined. The coupling reaction of substrate 9 with alkyl Grignard reagents (Scheme 5, 10b-l) resulted in higher yields than with aryl Grignard reagents (Scheme 5, 10m-q).

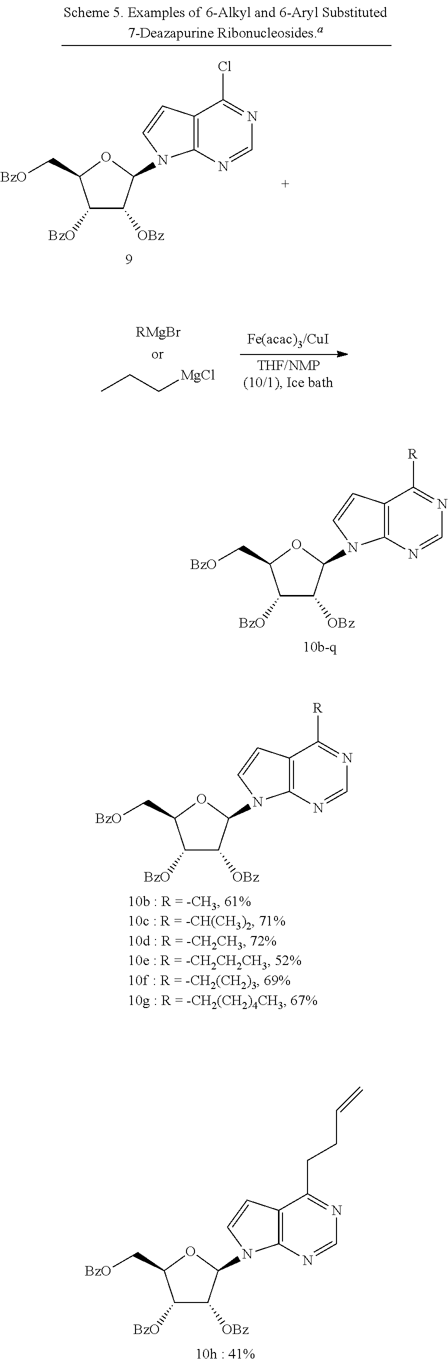

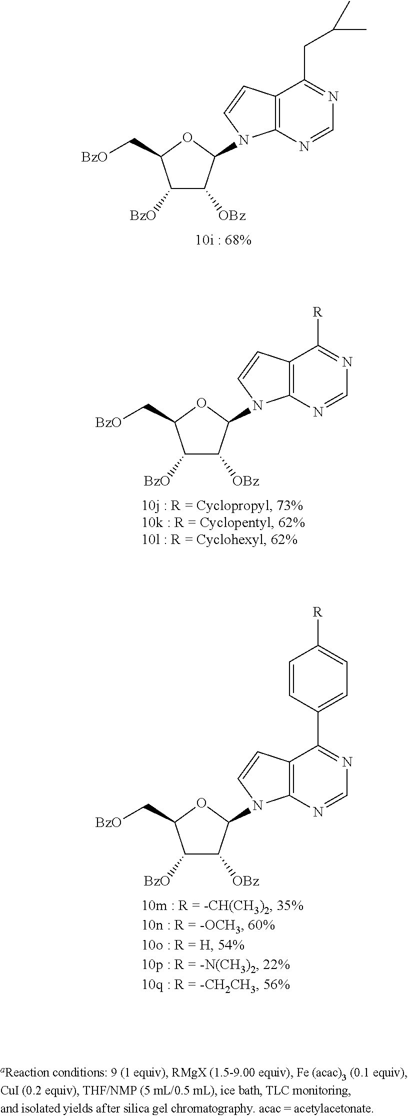

Prompted by the successful cross-coupling condition using the Fe(acac)₃/CuI combination, the synthesis of 6-aryl-7-deazapurine and 6-alkyl-7-deazapurine was revised using the Fe(acac)₃/CuI bimetallic system (Table 4). Likewise, improved yields were obtained for the synthesis of compound 5a, 5c, 5d, 5h and 5j by using this catalyst.

TABLE 4
Comparison of FeCl₃ versus Fe(acac)₃/Cul Catalyst for the Synthesis of
6-Substituted 7-Deazapurine^{a}

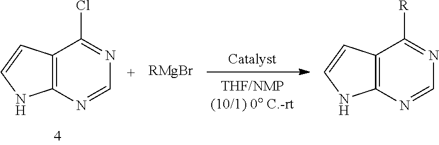

| Entry | Catalyst | Product | Yield |
|---|---|---|---|
| 1 | FeCl₃ | 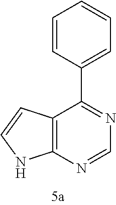 [SMILES: c1ccc(-c2ncnc3[nH]ccc23)cc1] | 65% |
| 2 | Fe(acac)₃/Cul | 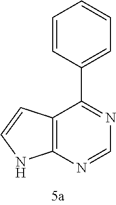 [SMILES: c1ccc(-c2ncnc3[nH]ccc23)cc1] | 75% |
| 3 | FeCl₃ | 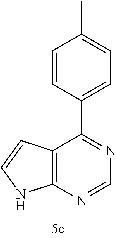 [SMILES: Cc1ccc(-c2ncnc3[nH]ccc23)cc1] | 68% |
| 4 | Fe(acac)₃/Cul | 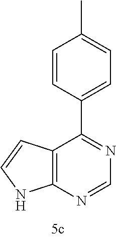 [SMILES: Cc1ccc(-c2ncnc3[nH]ccc23)cc1] | 76% |
| 5 | FeCl₃ | 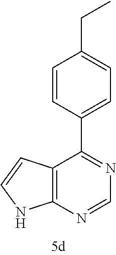 [SMILES: CCc1ccc(-c2ncnc3[nH]ccc23)cc1] | 50% |
| 6 | Fe(acac)₃/Cul | 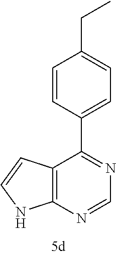 [SMILES: CCc1ccc(-c2ncnc3[nH]ccc23)cc1] | 62% |
| 7 | FeCl₃ | 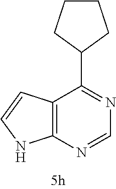 [SMILES: c1nc(C2CCCC2)c2cc[nH]c2n1] | 78% |
| 8 | Fe(acac)₃/Cul | 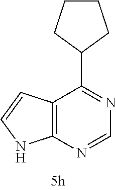 [SMILES: c1nc(C2CCCC2)c2cc[nH]c2n1] | 82% |
| 9 | FeCl₃ | 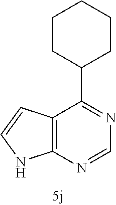 [SMILES: c1nc(C2CCCCC2)c2cc[nH]c2n1] | 73% |
| 10 | Fe(acac)₃/Cul | 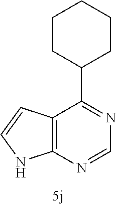 [SMILES: c1nc(C2CCCCC2)c2cc[nH]c2n1] | 76% |

^{a}Reaction conditions: 4 (1 equiv), RMgX (3-6 equiv), FeCl₃ or Fe(acac)₃ (0.1 equiv)/Cul (0.2 equiv), THF/NMP (5 mL /0.5 mL), ice bath to rt, TLC monitoring, and isolated yields after silica gel chromatography. acac = acetylacetonate.

The synthesis of compound 10f was used as a model reaction to evaluate the influence of CuI on the Fe-catalyzed cross-coupling reaction. Three reaction conditions were carried out. Compound 10f was first synthesized by using Fe(acac)₃ and pentylmagnesium bromide in the absence of CuI in 58% yield and a light brown precipitate was formed. The yield to obtain compound 10f increased to 70% by addition of 20% mol CuI in the reaction mixture and a dark brown precipitate was formed. When substrate 9 reacted with Gilman reagent which was prepared according to literature (Mizota, I. et al. Org. Lett. 2019, 21 (8), 2663-2667) from pentylmagnesium bromide (2 equiv) and CuI (1.2 equiv) in THF at −78° C., only 32% of compound 10f was obtained and a black precipitate was formed in the reaction mixture.

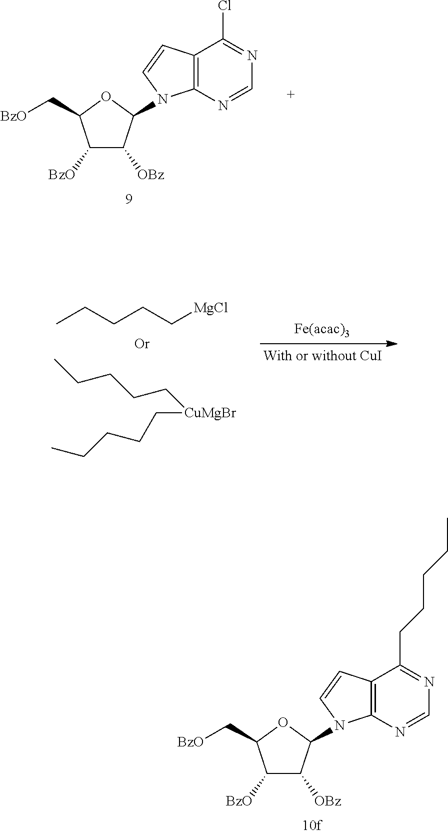

In the Fe-catalyzed Grignard cross-coupling, Kochi proposed an Fe(I)/Fe(II) mechanistic cycle (Smith, R. S. et al. J. Org. Chem. 1976, 41, 502), the active Fe(I) is formed by reduction of Fe(III) precatalyst by Grignard reagent. Later, Fürstner proposed a Fe(II)/Fe(0) mechanistic cycle (Fürstner, A. et al. J. Am. Chem. Soc. 2002, 124 (46), 13856-13863).

Based on a similar reaction to synthesize 1,1-diarylethylenes (Hamze, A. et al. Org. Lett. 2012, 14 (11), 2782-2785), it was assumed that the reaction proceeded through a similar mechanism, which formed an alkenyliron species, as seen in Scheme 6. Briefly, the oxidative addition of 2′,3′,5′-tri-O-benzoyl-6-chloro-9-β-D-ribofuranosyl-7-desazapurine (9) to a low-valent iron species A, generated by the reaction of Fe(acac)₃ with the Grignard reagent, would yield alkenyliron species B. Transmetallation with organocopper reagent forming di-organoiron species C followed by reductive elimination of the cross-coupling product regenerates low-valent iron species A.

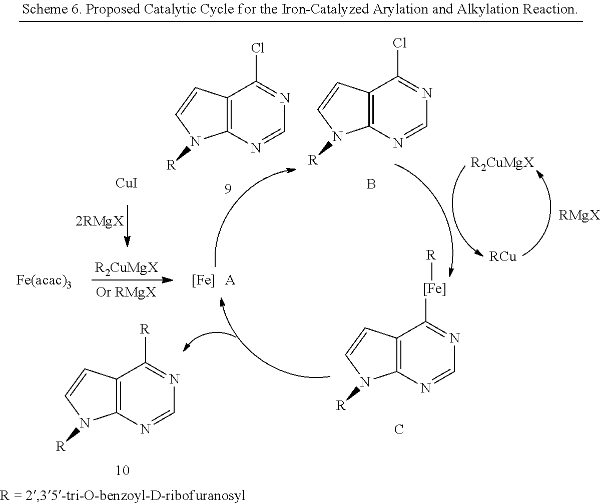

Finally, subsequent debenzoylation of compound 10a-q by treatment with 7 N ammonia in methanol yielded compound 11a-q, as seen in Scheme 7.

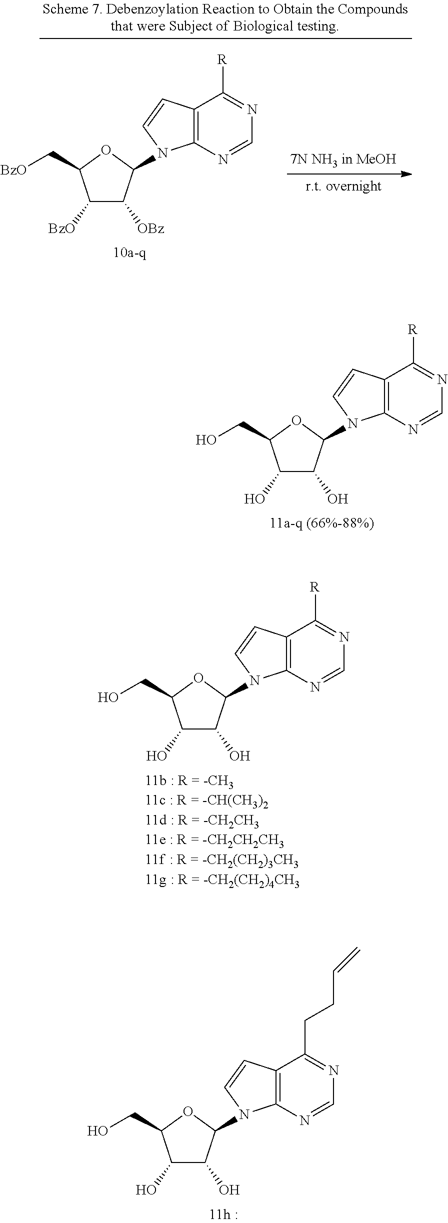

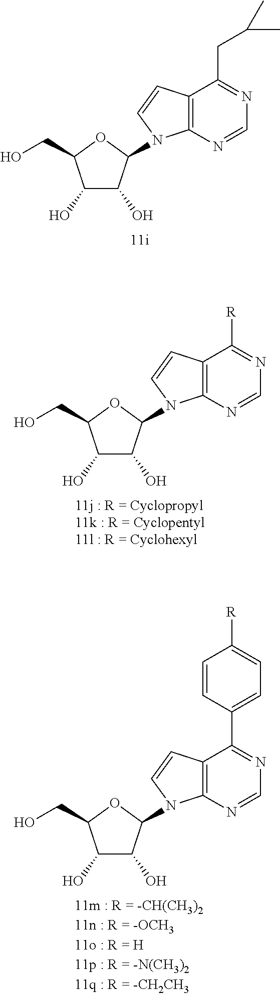

The novel compounds synthetized by the authors were tested for their antiviral activity.

### Antiviral Activity

The novel 6-Substituted 7-deazaadenosine analogues were screened for their ability to inhibit the in vitro replication of genogroup 1 NoV in HG23 NoV replicon cells. To this aim, quantitative reverse transcription polymerase chain reaction (intracecellular RNA)/β-actin (toxicity) assay was performed. All compounds were evaluated by performing dose response experiments in order to determine their EC₅₀ and EC₉₀ values. The related cytotoxicity (CC₅₀) was also determined in parallel in uninfected cells. All results obtained from these tests are summarized in Table 5.

Most of these compounds exhibited good to potent inhibitory activity against norovirus without significant cytotoxicity. In particular, compounds 11a and 11m emerged as the most potent compounds with an EC₅₀ of <0.0010 μM and 0.002 μM respectively, but with a lower EC₅₀/EC₉₀ ratio. 11d, 11e, 11c and 11l inhibited HuNoV replication with an EC₅₀ of 0.023 μM, 0.016 μM, 0.024 and 0.180 μM respectively, all of them had a better EC₅₀/EC₉₀ ratio and did not display significant cytotoxicity. 11b showed good activity against human norovirues with an EC₅₀ of 0.012 μM, it had a better EC₅₀/EC₉₀ ratio as well.

6-Substituted 7-deazaadenosine analogues displayed potent inhibitory activity in a HuNoV assay, especially for compounds 11c, 11e and 11l which showed potent activity against human norovirus and an excellent EC₅₀/EC₉₀ ratio without significant cytotoxicity.

TABLE 5
In vitro inhibitory activity of 6-substituted 7-deazaadenosine
analogues against human norovirus (HuNoV)

| | | EC₅₀ | EC₉₀ | CC₅₀ | | |
|---|---|---|---|---|---|---|
| Entry | Compound | (μM)^{a} | (μM)^{b} | (μM)^{c} | SI₅₀ | SI₉₀ |
| 1 | 11o | 0.211 | 61.9 | >100 | >474 | >2 |
| 2 | 11n | 0.094 | >100 | >100 | >1068 | 1 |
| 3 | 11f | 0.084 | >100 | >100 | >119 | 1 |
| 4 | 11a | 0.019 | >100 | >100 | >5291 | 1 |
| 5 | 11m | <0.0010 | 54.95 | >100 | >100000 | >2 |
| 6 | 11j | 0.205 | >100 | >100 | >487 | 1 |
| 7 | 11b | 0.012 | 0.068 | 0.530 | 46 | 8 |
| 8 | 11i | 0.002 | >100 | >100 | >52632 | 1 |
| 9 | 11d | 0.023 | 0.495 | 10.2 | 438 | 21 |
| 10 | 11e | 0.016 | 19.0 | >100 | >6274 | >5 |
| 11 | 11c | 0.024 | 2.69 | >100 | >4120 | >37 |
| 12 | 11g | 0.130 | 57.6 | 62.3 | 479 | 1 |
| 13 | 11k | >100 | >100 | >100 | 1 | 1 |
| 14 | 11q | 57.82 | >100 | >100 | >2 | 1 |
| 15 | 11p | 92.42 | >100 | >100 | >1 | >1 |
| 16 | 11l | 0.1802 | 7.24 | 42.42 | 235 | 6 |
| Control | 2’Cmethyl | 2.63 | 13.75 | >25 | >10 | >2 |
| | Cytidine | | | | | |

^{a}EC₅₀ is the effective concentration to inhibit the replication of the virus by 50%;
^{b}EC₉₀ is the effective concentration to inhibit the replication of the virus by 90%;
^{c}CC₅₀ is the cytotoxic concentration that reduces the number of viable cells by 50%.

In a similar experiment, the compounds were also screened for their ability to inhibit the in vitro replication of MERS Coronavirus strain EMC and Influenza A virus strain California/07/2009 in HG23 NoV replicon cells. All results obtained from these tests are summarized in Table 6. Most of the compounds did not exhibit any inhibitory activity against either virus. However, compound 11d did show a moderate inhibitory activity against both viruses, with an EC₅₀ of 0.42 μM for the Influenza A virus and an EC₅₀ of 9.2 μM for the MERS Coronavirus, respectively.

TABLE 6
In vitro inhibitory activity of 6-substituted 7-deazaadenosine
analogues against MERS Coronavirus strain EMC and Influenza
A virus strain California July 2009

| | | EC₅₀ | CC₅₀ | |
|---|---|---|---|---|
| Entry | Compound | (μM)^{a} | (μM)^{c} | SI₅₀ |
| Influenza | 11d | 0.42 | >100 | >240 |
| Control | Ribavirin | 3.6 | >1000 | >280 |
| MERS Coronavirus | 11d | 9.2 | >100 | >11 |
| Control | M128533 | 1.6 | >100 | >63 |

^{a}EC₅₀ is the effective concentration to inhibit the replication of the virus by 50%;
^{b}EC₉₀ is the effective concentration to inhibit the replication of the virus by 90%;
^{c}CC₅₀ is the cytotoxic concentration that reduces the number of viable cells by 50%.

### CONCLUSIONS

The cooperative metallic effect of FeCl₃ or Fe(acac)₃ that allows the formation of Csp²-Csp² and Csp²-Csp³ bonds by coupling 6-chloro-7-deazapurine and 6-chloro-7-deazapurine ribonucleoside was demonstrated with a series of functionalized Grignard reagents. The Fe(acac)₃/CuI combination has thus far not been employed as a catalytic system for cross-couplings of Grignard reagents with halogenated purine nucleosides. The condition-optimized reaction proved to be generic and chemoselective, presenting several advantages over known reactions not only because of the commercial availability and low cost of the catalysts, but also because of the mild conditions, experimental simplicity, and environmental friendliness. Additionally, the iron-catalyzed reaction eliminates known issues with the potential cellular toxicity of traces of palladium catalyst that could remain present in the final compounds to be tested biologically.

Of particular interest, cytotoxicity studies of compound 11a-q proved that modification of the 6-position of 7-deazapurine nucleosides may render the compounds selective for certain cancer cell lines. Surprisingly, 6-Substituted 7-deazaadenosine analogues displayed potent inhibitory activity in an HuNoV assay, especially for compounds 11c, 11e and 11l, which showed potent activity against human norovirus and an excellent EC50/EC90 ratio without significant cytotoxicity.

1. A method for prevention or treatment of a viral infection in a mammal, comprising providing to said mammal a therapeutically effective amount of a compound with general formula (A) or a pharmaceutically acceptable salt thereof, wherein in general formula (A) 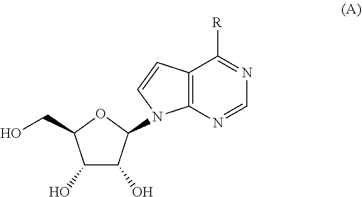
R is: (i) a C2-6alkyl group; (ii) a cycloalkyl group; (iii) an aryl group; (iv) an alkylaryl group; (v) an alkoxyaryl group; or (vi) an alkylaminoaryl group.

2. (canceled)

3. The method according to claim 1, wherein the viral infection is of an RNA-virus.

4. The method according to claim 1, wherein the mammal is a human.

5. The method according to claim 1, wherein the viral infection is of a Human Norovirus (HuNoV).

6. The method according to claim 5, wherein the Human Norovirus belongs to the Human Norovirus Genogroup 1.

7. (canceled)

8. (canceled)

9. The method according to claim 1, wherein in general formula (A), R is an alkylaryl group.

10. The method according to claim 9, wherein the alkyl group in the alkylaryl group is a C1-3 alkyl group.

11. The method according to claim 1, wherein the compound is selected from the group:

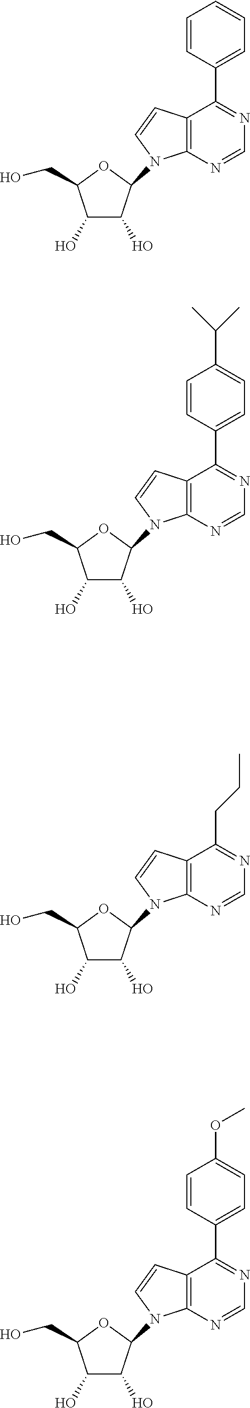 [SMILES: OC[C@H]1O[C@@H](n2ccc3c(-c4ccccc4)ncnc32)[C@H](O)[C@@H]1O ; CC(C)c1ccc(-c2ncnc3c2ccn3[C@@H]2O[C@H](CO)[C@@H](O)[C@H]2O)cc1 ; CCCc1ncnc2c1ccn2[C@@H]1O[C@H](CO)[C@@H](O)[C@H]1O ; COc1ccc(-c2ncnc3c2ccn3[C@@H]2O[C@H](CO)[C@@H](O)[C@H]2O)cc1]

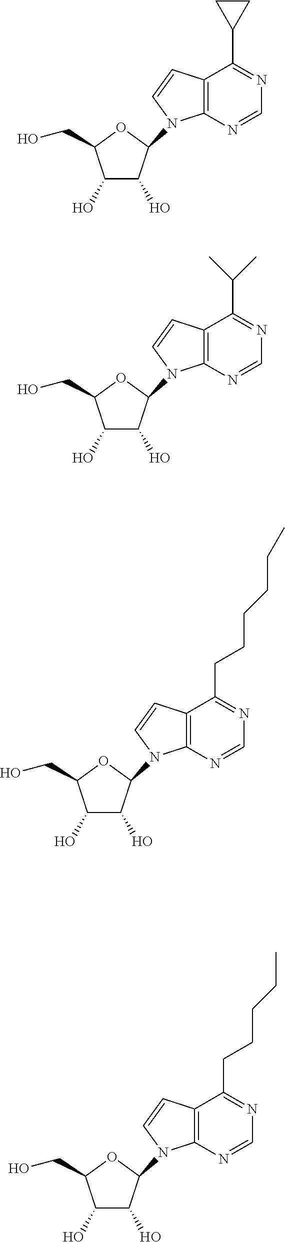 [SMILES: OC[C@H]1O[C@@H](n2ccc3c(C4CC4)ncnc32)[C@H](O)[C@@H]1O ; CC(C)c1ncnc2c1ccn2[C@@H]1O[C@H](CO)[C@@H](O)[C@H]1O ; CCCCCCc1ncnc2c1ccn2[C@@H]1O[C@H](CO)[C@@H](O)[C@H]1O ; CCCCCc1ncnc2c1ccn2[C@@H]1O[C@H](CO)[C@@H](O)[C@H]1O]

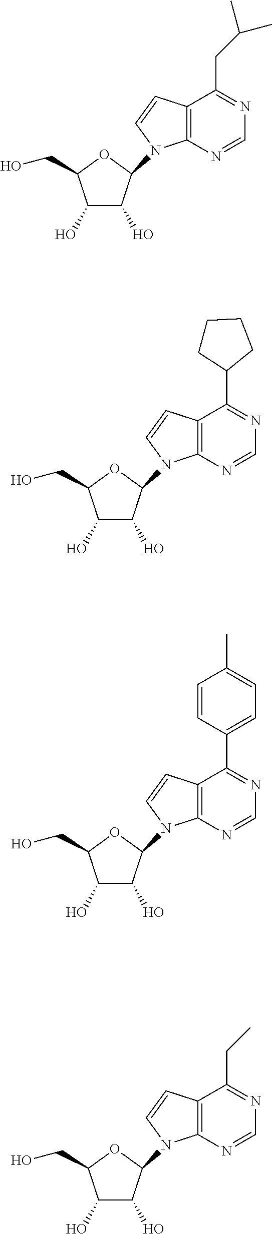 [SMILES: CC(C)Cc1ncnc2c1ccn2[C@@H]1O[C@H](CO)[C@@H](O)[C@H]1O ; OC[C@H]1O[C@@H](n2ccc3c(C4CCCC4)ncnc32)[C@H](O)[C@@H]1O ; Cc1ccc(-c2ncnc3c2ccn3[C@@H]2O[C@H](CO)[C@@H](O)[C@H]2O)cc1 ; CCc1ncnc2c1ccn2[C@@H]1O[C@H](CO)[C@@H](O)[C@H]1O]

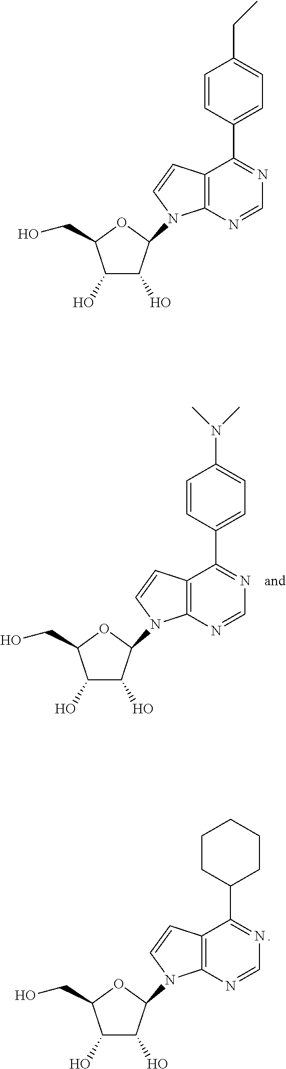 [SMILES: CCc1ccc(-c2ncnc3c2ccn3[C@@H]2O[C@H](CO)[C@@H](O)[C@H]2O)cc1 ; CN(C)c1ccc(-c2ncnc3c2ccn3[C@@H]2O[C@H](CO)[C@@H](O)[C@H]2O)cc1 ; OC[C@H]1O[C@@H](n2ccc3c(C4CCCCC4)ncnc32)[C@H](O)[C@@H]1O]

12. The method according to claim 1, wherein the compound has the formula (B):

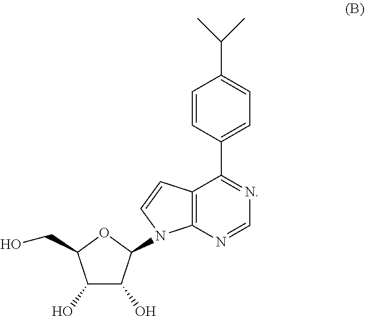 [SMILES: CC(C)c1ccc(-c2ncnc3c2ccn3[C@@H]2O[C@H](CO)[C@@H](O)[C@H]2O)cc1]

13. The method according to claim 1, wherein the compound has the formula (C):

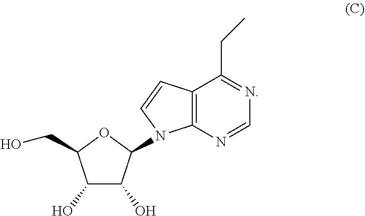 [SMILES: CCc1ncnc2c1ccn2[C@@H]1O[C@H](CO)[C@@H](O)[C@H]1O]

14. The method according to claim 13, wherein the vis viral infection is of a MERS coronavirus.

15. The method according to claim 13, wherein the viral infection is of influenza A.

16. A method for prevention or treatment of a viral infection in a mammal, comprising providing to said mammal a therapeutically effective amount of a pharmaceutical composition, wherein said pharmaceutical composition comprises:
(i) a therapeutically effective amount of a compound of general formula (A) and/or a pharmaceutical acceptable addition salt thereof, wherein 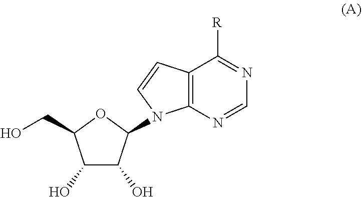
R is: (i) a C2-6alkyl group; (ii) a cycloalkyl group; (iii) an aryl group; (iv) an alkylaryl group; (v) an alkoxyaryl group; or (vi) an alkylaminoaryl group; and
(ii) at least one pharmaceutically acceptable carrier.

17. (canceled)

18. (canceled)

19. (canceled)

20. (canceled)

21. A method for synthesizing purine modified nucleoside analogues comprising a cross-coupling reaction of aryl or alkyl Grignard reagents with halogenated purine nucleosides, wherein the catalyst in the cross-coupling reaction is:
(i) iron; or
(ii) an iron/copper mixture.

22. The method of claim 21, wherein the purine modified nucleoside analogue is a pyrrolopyrimidine modified nucleoside analogue.

23. A method for prevention or treatment of a viral infection in a mammal, comprising providing to said mammal a therapeutically effective amount of a compound or its pharmaceutically acceptable salt thereof, wherein the compound is: 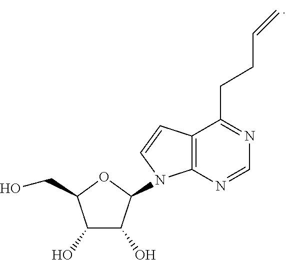 [SMILES: C=CCCc1ncnc2c1ccn2[C@@H]1O[C@H](CO)[C@@H](O)[C@H]1O]
